(19) United States
(12) Patent Application Publication
(10) Pub. No.: US 2022/0306603 A1
(43) Pub. Date: Sep. 29, 2022

(54) TARGETING DNA REPAIR IN TUMOR CELLS VIA INHIBITION OF ERCC1-XPF

(71) Applicant: The Governors of the University of Alberta, Edmonton (CA)

(72) Inventors: Frederick WEST, Edmonton (CA); Jack TUSZYNSKI, Edmonton (CA); Michael WEINFELD, Edmonton (CA); Khaled Hassan BARAKAT, Edmonton (CA); Ahmed H. ELMENOUFY, Edmonton (CA); Francesco GENTILE, North Vancouver (CA); Feridoun KARIMI-BUSHERI, Edmonton (CA); Claudia WEILBEER, Berrnburg (DE)

(21) Appl. No.: 17/596,491

(22) PCT Filed: Jun. 12, 2020
(86) PCT No.: PCT/CA2020/050823

§ 371 Date: Dec. 10, 2021

Related U.S. Application Data
(60) Provisional application No. 62/860,328, filed on Jun. 12, 2019.

(51) Int. Cl. C07D 401/12; C07D 401/14; C07D 405/14; C07D 409/14; C07D 471/04; C07D 413/14

(52) U.S. Cl. CPC C07D 401/12; C07D 401/14; C07D 405/14; C07D 409/14; C07D 471/04; C07D 413/14

(57) ABSTRACT

The current application relates to pyronaridine or 6-chloro-2-methoxyacridine analogs having binding affinity for the ERCC1-XPF hetero-dimerization interface. The compounds can be used for targeting DNA repair in tumor cells via ER-CC1-XPF inhibition, therefore improving the therapeutic benefit of irradiation or other cancer treatment, or reducing the dosage and adverse effects associated with such treatment.

### CROSS REFERENCE TO RELATED APPLICATION

This application claims priority to U.S. Patent Application 62/860,328, filed Jun. 12, 2019, the entire contents of which is hereby incorporated by reference.

### FIELD

The present disclosure relates generally to targeting DNA repair in tumor cells via inhibition of ERCC1-XPF.

### BACKGROUND

The ERCC1-XPF heterodimer is a critical DNA repair endonuclease. It plays a pivotal role in nucleotide excision repair (NER) of bulky adducts and helix-distorting DNA lesions such as UV-induced pyrimidine-(6,4)-pyrimidone photoproducts (6-4PPs) and CPDs (1-4). ERCC1-XPF is also involved in DNA interstrand crosslink (ICL) repair (5) in cells treated with platinum-based and other chemotherapeutic agents such as cyclophosphamide and mitomycin C (MMC) (6). In a recent study, 72% of colorectal cancer patients showed positive ERCC1 protein expression which may be a useful marker for colorectal cancer patients (7). There is also evidence that ERCC1-XPF participates in DNA double-strand break (DSB) repair (8,9). It thus contributes significantly to the response of cancer cells to a range of DNA-damaging chemotherapeutic agents and radiotherapy. In the ERCC1-XPF complex structure, ERCC1 is considered to be catalytically inactive but rather regulates DNA-protein and protein-protein interactions, whereas the endonuclease activity is provided by XPF, which also contains an inactive helicase-like motif that is likely to be involved in protein-protein interactions and DNA binding (10,11).

ERCC1-XPF is an attractive target for drug design due to the availability of experimental structures and the presence of multiple sites that can be inhibited with small molecules to stop the activity of the endonuclease. Our earlier drug design studies focused on the XPA-ERCC1 interaction site (12) and the XPF active site (13), fostering the use of computer-aided drug design to develop DNA repair inhibitors (14). However, due to the specificity of the ERCC1-XPF interaction and its general involvement in all the ERCC1-XPF mediated repair processes, the dimerization interface is perhaps the most promising domain to target pharmacologically (4). Dimerization and localization of ERCC1 and XPF is essential for the enzyme's stability and endonuclease activity (15). The dimerization of C-terminal regions of ERCC1 and XPF is the key interaction to form a stable heterodimer. C-terminal regions dimerize through the interaction of their double helix-hairpin-helix (HhH2) motifs (16,17). It is thought that XPF acts as a scaffold for ERCC1 during protein folding, and ERCC1 may exhibit improper folding in vitro in the absence of XPF (17). It was demonstrated that without dimerization the activity of ERCC1-XPF was abolished because neither protein was stable, and therefore they were rapidly degraded (18,19). As a result, development of small molecule inhibitors that can disrupt the HhH2 domain interactions between ERCC1 and XPF would be expected to sensitize cancer cells to chemotherapeutic treatments whose DNA-damaging effects are repaired by ERCC1-XPF-dependent pathways (4). Moreover, rational drug design methodology can be employed due to the availability of multiple experimental structures of the dimerized HhH2 domains (for example, PDB code 2A1J and 1Z00) (19).

Recently, McNeil et al. employed an in-silico screening approach targeting three sites on the XPF HhH2 domain to identify possible inhibitors for the dimer. They discovered a small molecule able to inhibit the NER activity in melanoma cells and slightly sensitize them to cisplatin treatment. However, the reported K_{d} and IC₅₀ values for this compound were suboptimal, in the medium-high μM range (20). In addition, Chapman et al. (21,22), and Arora et al. (23) identified and optimized different small molecules targeting the active site on the XPF nuclease domain. These efforts resulted in several endonuclease inhibitors with IC₅₀ in the nanomolar range, able to diminish NER activity and enhance the cytotoxicity of platinum-based drugs in cancer cells. Although the specificity of these inhibitors to the ERCC1-XPF endonuclease was assessed in some cases, similarities among active sites of various endonucleases could produce off-target interactions from these compounds; the lack of structural insights of the ligand-protein complexes in this case also limits further development of this series. Most recently, Yang et al. proposed the cellular delivery of therapeutic peptides mimicking the ERCC1 HhH2 domain (residues 220-297) as a promising alternative strategy to inhibit NER activity and sensitize cancer cells to DNA-damaging agents (24).

Development of small molecule inhibitors of the HhH2 domain interaction would be expected to sensitize cells to therapies whose DNA-damaging effects are repaired by ERCC1-XPF-dependent pathways. Jordheim et al, focused on developing small molecule inhibitors of the NER pathway acting through the inhibition of ERCC1-XPF heterodimerization and reported that F06 (also called NSC-130813 or NER102) interacted with XPF, repressed the interaction between ERCC1 and XPF in vitro and sensitized cancer cells to MMC and cisplatin (25). This affirms that targeting this protein-protein interaction can enhance the cytotoxic activity of crosslinking agents such as cisplatin. The initial hit, F06, arose from a virtual screening (VS) of a large compound library, and the study also provided a characterization of the XPF binding pocket and the binding mode of the compound to it (25). F06 was predicted to interfere with the heterodimerization of ERCC1 and XPF, a necessary step to attain DNA repair activity.

Preliminary in-vitro assays confirmed that F06 shows promising inhibitory activity and acts synergistically with cisplatin. However, the activity of F06 is suboptimal in terms of clinical properties including potency and safety, and a derivatization strategy, suggested by Jordheim et al (25), was adapted to optimize the action of the compound. Also, previous efforts on developing inhibitors for ERCC1-XPF activity were carried out on the truncated version of the heterodimer, and not the full-length protein (2,25-27).

### SUMMARY

In one aspect there is described, a compound of:

(a) Formula (I),

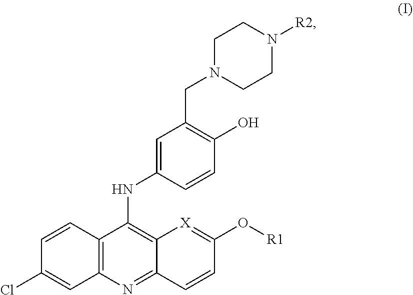

or a tautomer, a pharmaceutically acceptable salt, a solvate, or a functional derivative thereof;

wherein:

R¹ is H, short chain alkyl (Me, Et, iPr, nPr, nBu, iBu), substituted or unsubstituted phenyl, substituted or unsubstituted benzyl;

R² is a short chain alkyl (Me, Et, iPr, nPr, nBu, iBu), CH₂CH₂N(CH₃)₂, or CH₂CH₂N(CH(CH₃)₂)₂ substituted or unsubstituted phenyl, or substituted or unsubstituted benzyl, or C(Y)((CH₂)ₙ)N(R³R⁴), where n is 0, 1, 2, 3, or 4, and Y is O or S, and R³ and R⁴ are independently H, a short chain alkyl (Me, Et, iPr, nPr, nBu, iBu), substituted or unsubstituted phenyl, substituted or unsubstituted benzyl, or collectively form a 4-membered heterocyclic ring, a 5-membered heterocyclic ring, or a 6-membered heterocyclic ring, or C((CH₂)ₙ)N(R³R⁴), where n is 0, 1, 2, 3, 4, or 5, and R³ and R⁴ are independently H, a short chain alkyl (Me, Et, iPr, nPr, nBu, iBu), substituted or unsubstituted phenyl, substituted or unsubstituted benzyl, or collectively form a 4-membered heterocyclic ring, a 5-membered heterocyclic ring, or a 6-membered heterocyclic ring;

X is either C or N; or

wherein when either R₁ or R₂ is an unbranched alkyl, the other is not an unbranched alkyl;

(b) Formula (II):

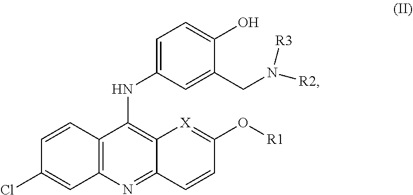

or a tautomer, a pharmaceutically acceptable salt, a solvate, or a functional derivative thereof;

wherein:

R¹ is H, a short chain alkyl (Me, Et, iPr, nPr, nBu, iBu), substituted or unsubstituted phenyl, substituted or unsubstituted benzyl;

R² and R³ are independently a short chain alkyl (Me, Et, iPr, nPr, nBu, iBu), substituted or unsubstituted phenyl, substituted or unsubstituted benzyl, or collectively form a 4-membered heterocyclic ring, a 5-membered heterocyclic ring, or a 6-membered heterocyclic ring;

X is either C or N; or

wherein when either R₁ or R₂ is an unbranched alkyl, the other is not an unbranched alkyl; or

(c) formula (III)

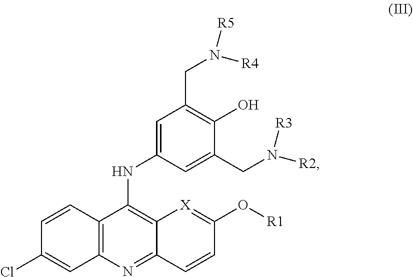

or a tautomer, a pharmaceutically acceptable salt, a solvate, or a functional derivative thereof;

wherein:

R¹ is H, a short chain alkyl (Me, Et, iPr, nPr, nBu, iBu), substituted or unsubstituted phenyl, substituted or unsubstituted benzyl;

R² and R³ are independently a short chain alkyl (Me, Et, iPr, nPr, nBu, iBu), substituted or unsubstituted phenyl, substituted or unsubstituted benzyl, or collectively form a 4-membered heterocyclic ring, a 5-membered heterocyclic ring, or a 6-membered heterocyclic ring;

R⁴ and R⁵ are independently a short chain alkyl (Me, Et, iPr, nPr, nBu, iBu), substituted or unsubstituted phenyl, substituted or unsubstituted benzyl, or collectively form a 4-membered heterocyclic ring, a 5-membered heterocyclic ring, or a 6-membered heterocyclic ring;

X is either C or N; and

wherein when R² and R³ collectively form a heterocyclic ring, R⁴ and R⁵ collectively do not form a heterocyclic ring.

Me is a methyl group, Et is an ethyl group, iPr is an isopropyl group, nPr is a straight chain/unbranched propyl group, nBu is a straight chain/unbranched butyl group, and iBu is an isobutyl group.

In one example, comprising:

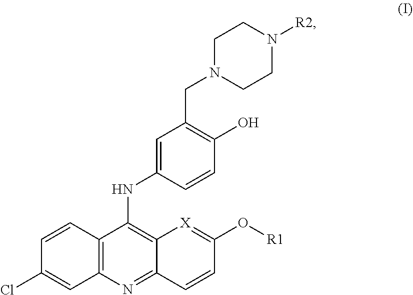

wherein R¹ is H, or CH₃

wherein R² is CH₃, CH₂CH₂N(CH₃)₂, or CH₂CH₂N(CH(CH₃)₂)₂, and

wherein X is C or N and

wherein when either R¹ or R² is CH₃, the other is not CH₃, or

a tautomer, or a pharmaceutically acceptable salt, or a solvate, or a functional derivative thereof.

In one example, wherein R¹ is CH₃, R² is H and X is C.

In one example, wherein R¹ is CH₂CH₂N(CH₃)₂, R² is H, and X is C.

In one example, wherein R¹ is CH₂CH₂N(CH₃)₂, R² is CH₃, and X is C.

In one example, wherein R¹ is CH₂CH₂N(CH(CH₃)₂)₂, R² is CH₃, and X is C.

In one example, wherein CH₂CH₂N(CH₃)₂, R² is CH₃, and X is N

In one aspect there is provided a method of treating a subject having cancer, or suspected of having cancer, comprising, administering a therapeutically effective amount of a compound of:

(a) Formula (I),

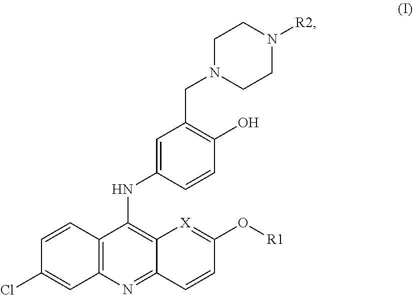

or a tautomer, a pharmaceutically acceptable salt, a solvate, or a functional derivative thereof;

wherein:

R¹ is H, short chain alkyl (Me, Et, iPr, nPr, nBu, iBu), substituted or unsubstituted phenyl, substituted or unsubstituted benzyl;

R² is a short chain alkyl (Me, Et, iPr, nPr, nBu, iBu), CH₂CH₂N(CH₃)₂, or CH₂CH₂N(CH(CH₃)₂)₂ substituted or unsubstituted phenyl, or substituted or unsubstituted benzyl, or C(Y)((CH₂)ₙ)N(R³R⁴), where n is 0, 1, 2, 3, or 4, and Y O or S, and R³ and R⁴ are independently H, a short chain alkyl (Me, Et, iPr, nPr, nBu, iBu), substituted or unsubstituted phenyl, substituted or unsubstituted benzyl, or collectively form a 4-membered heterocyclic ring, a 5-membered heterocyclic ring, or a 6-membered heterocyclic ring; or C((CH₂)ₙ)N(R³R⁴), where n is 0, 1, 2, 3, 4, or 5, and R³ and R⁴ are independently H, a short chain alkyl (Me, Et, iPr, nPr, nBu, iBu), substituted or unsubstituted phenyl, substituted or unsubstituted benzyl, or collectively form a 4-membered heterocyclic ring, a 5-membered heterocyclic ring, or a 6-membered heterocyclic ring;

X is either C or N; or

wherein when either R₁ or R₂ is an unbranched alkyl, the other is not an unbranched alkyl;

(b) Formula (II):

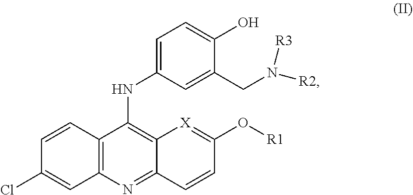

or a tautomer, a pharmaceutically acceptable salt, a solvate, or a functional derivative thereof;

wherein:

R¹ is H, a short chain alkyl (Me, Et, iPr, nPr, nBu, iBu), substituted or unsubstituted phenyl, substituted or unsubstituted benzyl;

R² and R³ are independently a short chain alkyl (Me, Et, iPr, nPr, nBu, iBu), substituted or unsubstituted phenyl, substituted or unsubstituted benzyl, or collectively form a 4-membered heterocyclic ring, a 5-membered heterocyclic ring, or a 6-membered heterocyclic ring;

X is either C or N; or

wherein when either R₁ or R₂ is an unbranched alkyl, the other is not an unbranched alkyl; or

(c) formula (III)

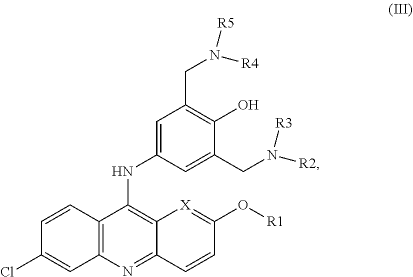

or a tautomer, a pharmaceutically acceptable salt, a solvate, or a functional derivative thereof;

wherein:

R¹ is H, a short chain alkyl (Me, Et, iPr, nPr, nBu, iBu), substituted or unsubstituted phenyl, substituted or unsubstituted benzyl;

R² and R³ are independently a short chain alkyl (Me, Et, iPr, nPr, nBu, iBu), substituted or unsubstituted phenyl, substituted or unsubstituted benzyl, or collectively form a 4-membered heterocyclic ring, a 5-membered heterocyclic ring, or a 6-membered heterocyclic ring;

R⁴ and R⁵ are independently a short chain alkyl (Me, Et, iPr, nPr, nBu, iBu), substituted or unsubstituted phenyl, substituted or unsubstituted benzyl, or collectively form a 4-membered heterocyclic ring, a 5-membered heterocyclic ring, or a 6-membered heterocyclic ring;

X is either C or N; and

wherein when R² and R³ collectively form a heterocyclic ring, R⁴ and R⁵ collectively do not form a heterocyclic ring.

In one example, comprising:

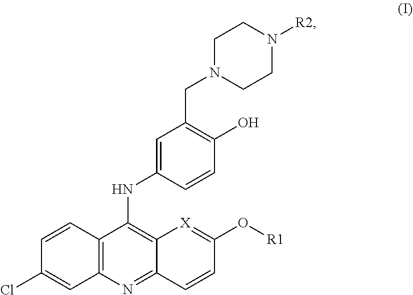

wherein R¹ is CH₃, CH₂CH₂N(CH₃)₂, or CH₂CH₂N(CH(CH₃)₂)₂, and
wherein R² is H, or CH₃,
wherein X is C or N

wherein when either R¹ or R² is CH₃, the other is not CH₃, or a tautomer, or a pharmaceutically acceptable salt, or a solvate, or a functional derivative thereof.

In one example, wherein R¹ is CH₃, and R² is H, and X is C.

In one example, wherein R¹ is CH₂CH₂N(CH₃)₂, and R² is H, and X is C.

In one example, wherein R¹ is CH₂CH₂N(CH₃)₂, and R² is CH₃, and X is C.

In one example, wherein R¹ is CH₂CH₂N(CH(CH₃)₂)₂, and R² is CH₃, and X is C.

In one example, wherein CH₂CH₂N(CH₃)₂, R² is CH₃, and X is N.

In one aspect there is provided a method of treating a subject having cancer, or suspected of having cancer, comprising, administering a therapeutically effective amount of a compound of Formula (IV),

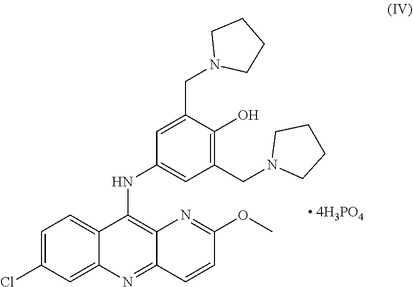 [SMILES: COc1ccc2nc3cc(Cl)ccc3c(Nc3cc(CN4CCCC4)c(O)c(CN4CCCC4)c3)c2n1]

or a pharmaceutically acceptable salt, or a solvate, or a functional derivative thereof.

In another aspect of the present application, there is provided a pharmaceutical composition comprising a compound as described herein, and a pharmaceutically acceptable carrier, diluent, or vehicle.

In another aspect of the present application, there is provided a use of a compound as described herein, or a composition as described herein for treating a subject having cancer, or suspected of having cancer.

In another aspect, there is provided a use of a compound as described herein, or a composition as described herein in the manufacture of a medicament for treating a subject having cancer, or suspected of having cancer.

In another aspect of the present application, there is provided a method of treating a subject having cancer, or suspected of having cancer, comprising, administering a therapeutically effective amount of a compound as described herein, or a composition as described herein.

In another aspect of the present application, there is provided a compound of as described herein, or a composition as described herein for inhibiting ERCC1-XFP.

In another aspect, there is provided a compound as described herein, or a composition as described herein for increasing the sensitivity of a cancerous cell of a subject to a chemotherapeutic agent or radiation therapy.

In another aspect of the present application, there is provided a method of chemosensitizing or radiosensitizing a cancerous cell in a subject in need of chemotherapy or radiation therapy, comprising: administering to said subject a compound as described herein, or a composition as described herein.

In another aspect of the present application, there is provided a kit comprising: a compound as described herein, or a composition as described herein, and a container, and/or instructions for the use thereof.

### BRIEF DESCRIPTION OF THE FIGURES

Embodiments of the present disclosure will now be described, by way of example only, with reference to the attached Figures.

FIG. 1. Analysis of the binding mode of the top hits. A) Per-residue decomposition of ligand-receptor binding energy. F06 and A3 had similar patterns, while A4 showed a different pattern of interaction energies. Blue cells indicate favorable interactions, while red cells indicate unfavorable interactions. Refer to the text for more details. B) Lowest potential energy snapshot extracted from the MD simulation of A4 bound to XPF. Hydrogen bonds are colored in purple. The highest pocket contributors in terms of total interaction energy are labelled. C) Interaction diagram for A4 and surrounding residues and water molecules. The non-bonded interactions established with water molecules was responsible for a highly-favorable polar solvation contribution to the binding energy. The diagram refers to the lowest potential energy snapshot extracted from the MD simulation. Original PDB structure for XPF: 1Z00.

FIG. 2. In-vitro inhibition of ERCC1-XPF endonuclease activity. A microplate fluorescent assay was used to measure inhibition of ERCC1-XPF endonuclease activity by the different compounds. Incubation of the DNA stem-loop substrate with ERCC1-XPF resulted in the release of a fluorescent 8-base fragment. A) Shows the increase in fluorescence (FRU) with time. A representative tracing of the effect of the different compounds (8 μM each) on the incision activity is shown. B) (inset) shows a representative plot of enzyme rate (ΔRFU/time) vs A4 concentration.

FIG. 3. Determination of the affinity (K_{d}) between A4 and the ERCC1-XPF complex. Quenching of the intrinsic protein fluorescence of the tryptophan residues was used to monitor the interaction between the compounds and the protein. A) Fluorescence of ERCC1-XPF (13 nM and 20 nM) incubated with A5 (negative control), and B) A4 (active inhibitor) respectively. C) Unimodal binding pattern and the binding affinity of A4 with ERCC1-XPF (70 nM). The protein was excited at 295 nm and fluorescence intensity was monitored at 330 nm (see inset). The fraction bound (i.e. relative fluorescence) intensity versus ligand concentration is plotted.

FIG. 4. Inhibition of cellular NER. A) Immunofluorescence images of the UV-based assay for detecting CPDs in HCT116 cells treated with compounds 4 and 5. B) Normalized fluorescence intensity of the treated cells (±S.E.M) based on quantitation of fluorescence from 50 randomly selected cells per treatment. All the values obtained for treatment with A4 from 4-24 h post-irradiation were significantly different (p<0.005, Student's t-test) from the values obtained with the control irradiated cells.

FIG. 5. Sensitization of cells to UV and cyclophosphamide. A) Survival of HCT116 cells exposed to increasing doses of 254 nm UV radiation and treated with 1 and 2 μM A4 determined by the clonogenic survival assay. B) Survival of HCT116 cells exposed to increasing doses of cyclophosphamide and treated with 1 and 2 μM A4 determined by the clonogenic survival assay. The dashed line indicates that the no colonies were observed after treatment with 300 μM cyclophosphamide+2 μM A4. The survival curves (±S.E.M) are based on three independent sets of determinations. All the values obtained with cells treated with 2 μM A4 were significantly different (p<0.005, Student's t-test) from the values obtained with the control cells not treated with A4.

FIG. 6. A) One-pot sequential addition reaction for synthesis of compounds 1-8. B) Main steps and intermediates included in the one pot sequential addition reaction.

FIG. 7. Visual analysis of the F06 docking pose. A) Shape complementarity between the docked pose of F06 and the XPF binding site. The compound was docked to the XPF pocket that wraps around residue F293 of ERCC1 in the dimerization complex. B) Non-bonded interactions. Observed hydrogen bonds are represented in purple. Residues involved in hydrogen bonding or hydrophobic interactions with the ligand are explicitly represented. See text for more details. C) Affinity mapping of the F06 binding site on the XPF HhH2 domain. Blue color indicates zones of interaction where a donor probe atom shows a potential of at least −2 kcal/mol, therefore where a ligand donor atom will contribute favourably to the free energy of binding. Red indicates acceptor favorable zones, and white hydrophobic favorable zones. D) Pharmacophore model designed for the lead compound binding to the XPF binding site. Aro features are aromatic, Acc are hydrogen bond acceptors and Don hydrogen bond donors. Original PDB structure for XPF: 1Z00.

FIG. 8. Cell proliferation of HCT116 cells treated with compounds 4 (active inhibitor) and 5 (non-active inhibitor) using the MTS assay.

FIG. 9. Structure of F06 hit and the potential modification sites for hit to lead optimisation.

FIG. 10. IC50 value of 0.167+/−0.028 μM for compound Gen C1.

FIG. 11. In-vitro inhibition of ERCC1-XPF endonuclease activity. A microplate fluorescent assay was used to measure inhibition of ERCC1-XPF endonuclease activity by the different compounds. Incubation of the DNA stem-loop substrate with ERCC1-XPF resulted in the release of a fluorescent 8-base fragment accompanied by an increase in fluorescence (RFU) with time. A) A representative tracing of the effect of the different compounds (10 μM each) on the incision activity is shown. B) Plot of enzyme rate (ΔRFU/time) vs Gen B9 concentration.

FIG. 12 ERCC1-XPF endonuclease assay of Compounds Gen C-1, 3, 6, 10, Gen A-4 (TFA salt), and control Gen C-7 and 10.

FIG. 13. ERCC1-XPF endonuclease assay of compounds Gen C-1, 5, 9, 10, 11, A-4, Gen B-9, and Gen B-1.

FIG. 14. Crystal violet based viability assay to assess the cytotoxicity profile of A) Gen B9 (active inhibitor) and Gen B7 (negative control), and B) F06 (hit).

FIG. 15. Cytotoxicity assay using crystal violet staining (A) and colony forming assay.

FIG. 16. Sensitization of HCT116 cells to UV and cyclophosphamide. A) Cellular survival upon exposure to increasing doses of 254 nm UV radiation and treated with 2 and 4 μM Gen B7, B) 2 μM F06, and C) 2 and 4 μM Gen B9 determined by the clonogenic survival assay. D) HCT116 cell survival following exposure to increasing doses of cyclophosphamide and treated with 4 and 2 μM of Gen B7 and B9 respectively, determined by the clonogenic survival assay. The cell survival curves (±SEM) are based on three independent sets of experiments. All the values related to cells treated with 2 μM Gen B9 were significantly different (p<0.005, Student's test) from the values obtained with the control cells not treated with Gen B9.

FIG. 17. Sensitization of HCT116 cells to UV and cyclophosphamide. Cellular survival upon exposure to increasing doses of 254 nm UV radiation and treated with 0.5 and 1 μM Gen C1 (A and B). HCT116 cells survival following exposure to increasing doses of cyclophosphamide and treated with 1 μM of Gen C-1 and Gen C-5, determined by the clonogenic survival assay (C).

FIG. 18. Inhibition of cellular NER by compound B9. (A) Immunofluorescence images of the UV-based assay for detecting CPDs in HCT116 colorectal cancer cells treated with compounds B9 (at 2 μM) and compared to negative control B7 and the control. (B) Normalized fluorescence intensity of the treated cells (±SEM) based on quantitation of fluorescence from 100 randomly selected cells per treatment. Treated cells with active inhibitor B9 were significantly different from the non-inhibitory negative control B7 from 4 to 24 h postirradiation (p<0.005, Student's t test).

FIG. 19. Immunocytochemistry Assay of Gen C-1 on HCT116 Cells. Normalized fluorescence intensity of the treated cells.

FIG. 20. Six synthesized quinoline derivatives and pyronaridine (D-4).

FIG. 21. Inhibition of XPF-ERCC1 endonuclease activity of different quinoline derivatives (and lead compounds.

FIG. 22. Toxicity, UV repair, UV survival and cyclophosphamide toxicity of F06 and the lead derivatives. (NDT: Not determined).

FIG. 23. Summary of compounds Gen C.

FIG. 24. In silico screening for Gen C: Average binding energies were calculated over a molecular dynamics trajectory using MM/GBSA method; cLog P values were determined in MOE using an empirical method based on single atom contributions.

### DETAILED DESCRIPTION

Generally, the present disclosure provides targeting DNA repair in tumor cells via inhibition of ERCC1-XPF.

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) and/or ingredient(s) as appropriate.

In one aspect, there is described compounds, compositions, methods and kits for treating a subject suspect of having cancer or having cancer.

In one aspect, there is described compounds, compositions, methods and kits for increasing sensitivity of cells and/or tumours to chemotherapeutic agents and/or ionizing radiation.

As further described herein, in one aspect the present application relates to inhibitors of ERCC1-XPF.

As used herein, the term “inhibit” with respect to ERCC1-XPF is intended to include partial or complete inhibition of ERCC1-XPF activity.

The term “radiosensitizer”, as used herein refers to an agent, molecule, compound or composition that enhances the sensitivity of a neoplastic cell, a cancer cell and/or a tumour to the effects of radiation. The “sensitivity” of a neoplastic cell, a cancer cell, and/or a tumour to radiation is the susceptibility of the neoplastic cell, cancer cell, and/or tumour to the inhibitory effects of radiation on the cell's or tumour's growth and/or viability.

The term “chemosensitizer”, as used herein, refers to an agent, molecule, compound or composition that enhances the sensitivity of a neoplastic cell, a cancer cell and/or a tumor to the effects of a chemotherapeutic agent. The “sensitivity” of a neoplastic cell, a cancer cell, and/or a tumour to a chemotherapeutic agent is the susceptibility of the neoplastic cell, cancer cell, and/or tumour to the inhibitory effects of a chemotherapeutic agent on the cell's or tumour's growth and/or viability.

The term “subject”, as used herein, refers to any human or non-human animal whom would benefit from treatment with a chemosensitizer and/or a radiosensitizer, and/or has a disorder associated with ERCC1-XPF. Non-limiting examples of a subject include humans, non-human mammal, primates, rodents, companion animals (including but not limited to dogs, cats, mice, rats), livestock (including but not limited to horses, sheep, cattle, pigs), reptiles, amphibians, and the like. In a specific example, the subject is a human.

The term “cancer” refers to or describes the physiological conditions in a subject generally characterized by inappropriate cellular proliferation, abnormal or excessive cellular proliferation. Cancers may be solid or non-solid cancers. Cancers may be a primary cancer and/or metastatic cancer. Cancers include, but are not limited to, a solid cancer, a non-solid cancer, a primary cancer, a metastatic cancer, lung cancer, bladder cancer, cervical cancer, testicular cancer, ovarian cancer, head and neck cancer, endometrial cancer, breast cancer, central nervous system or germ cell tumors, osteogenic sarcoma, colon cancer, rectal cancer, Hodgkin's and non-Hodgkin's lymphoma, Burkitt's lymphoma, chronic lymphocytic leukemia, chronic myelocytic leukemia, acute myeloid leukemia, acute lymphocytic leukemia, t-cell lymphoma, multiple myeloma, neuroblastoma, retinoblastoma, stomach cancer, pancreatic cancer, esophageal cancer, or anal cancer.

The term “sample” as used herein encompasses a variety of cell-containing bodily fluids and/or secretions as well as tissues including, but not limited to, a cell(s), tissue, whole blood, blood-derived cells, plasma, serum, sputum, mucous, bodily discharge, and the like, and combinations thereof. Methods of obtaining such samples from subject are known to the skilled worker.

The term “treatment”, “treat”, “treating”, or lessening of severity” as used herein, refers to obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment.

The term “amelioration” or “ameliorates” as used herein refers to a decrease, reduction or elimination of a condition, disease, disorder, or phenotype, including an abnormality or symptom.

In a specific example, treatment is carried out in vivo.

In a specific example, treatment is carried out in vitro, including but not limited to, in test tube, in cultured cells (both adherent cells and non-adherent cells), and the like.

In a specific example, treatment is carried out ex vivo, including but not limited to, in test tube, in cultured cells (both adherent cells and non-adherent cells), and the like.

The term “prognosis” as used herein refers to the prediction of the likelihood of cancer-attributable death or progression, including recurrence, metastatic spread, and drug resistance, of a neoplastic disease.

The term “pharmaceutically effective amount” as used herein refers to the amount of a drug or pharmaceutical agent that will elicit the biological or medical response of a tissue, system, animal or human that is being sought by a researcher or clinician. This amount can be a therapeutically effective amount.

The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” as used herein refers to a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation, for example the carrier does not decrease the impact of the agent on the treatment. In other words, a carrier is pharmaceutically inert. The terms “physiologically tolerable carriers” and “biocompatible delivery vehicles” are used interchangeably. Thus, the term “carrier” or “excipient” may refer to a non-toxic solid, semi-solid or liquid filler, diluent. The term includes solvents, dispersion, media, coatings, isotonic agents, and adsorption delaying agents, and the like. The use of such media and agents for pharmaceutically active substances is well known in the art.

As used herein, the term “pharmaceutically-acceptable salts” refers to the conventional nontoxic salts or quaternary ammonium salt. These salts can be prepared in situ in the administration vehicle or the dosage form manufacturing process, or by separately reacting a compound in its free base or acid form with a suitable organic or inorganic acid or base, and isolating the salt thus formed during subsequent purification. Conventional nontoxic salts include those derived from inorganic acids such as sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

A “pharmaceutical composition” as used herein refers to a chemical or biological composition suitable for administration to a mammalian subject. Such compositions may be specifically formulated for administration via one or more of a number of routes, including but not limited to, oral, parenteral, intravenous, intraarterial, subcutaneous, intranasal, sublingual, intraspinal, intracerebroventricular, and the like.

The term “functional derivative” as used herein refers to a molecule that retains a biological activity (either function or structural) that is substantially similar to that of the original compound. A functional derivative or equivalent may be a natural derivative or is prepared synthetically. For example, the term “derivatitive” as used herein may refer to a chemical substance related structurally to another, i.e., an “original” substance, which can be referred to as a “parent” compound. A “derivative” can be made from the structurally-related parent compound in one or more steps. The general physical and chemical properties of a derivative are also similar to the parent compound.

Also encompassed is prodrug or “physiologically functional derivative”. The term “physiologically functional derivative” as used herein refers to compounds which are not pharmaceutically active themselves but which are transformed into their pharmaceutically active form in vivo, i.e. in the subject to which the compound is administered. The term “prodrug” as used herein, refers to a derivative of a substance that, following administration, is metabolized in vivo, e.g. by hydrolysis or by processing through an enzyme, into an active metabolite.

As used herein, a “prodrug” refers to a compound that can be converted via some chemical or physiological process (e.g., enzymatic processes and metabolic hydrolysis). The term “prodrug” also refers to a precursor of a biologically active compound that is pharmaceutically acceptable. A prodrug may be inactive when administered to a subject, i.e. an ester, but is converted in vivo to an active compound, for example, by hydrolysis to the free carboxylic acid or free hydroxyl. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound may be prepared by modifying functional groups present in the active compound in such a way that the modifications are cleaved, either in routine manipulation or in vivo, to the parent active compound.

Described herein is the design, and synthesis of new compounds and the identification of inhibitors of ERCC1-XPF.

The term “enantiomer” is used to describe one of a pair of molecular isomers which are mirror images of each other and non-superimposable.

Radiation and many of the current chemotherapeutic agents used to treat cancer kill cancer cells by damaging the DNA in the cells. Because cells contain enzymes that repair DNA damage, small molecules have been developed that inhibit DNA repair enzymes like ERCC1-XPF and improve the ability of radiation and drugs to kill cancer cells. Compounds described herein can be used to treat cancers in combination with DNA crosslinking agents including, but not limited to, the platinum-based family of drugs (e.g., cisplatin, carboplatin, or oxaliplatin), cyclophosphamide, and mitomycin C.

Damage to cellular DNA is the principle cause of cell death induced by many chemotherapeutic drugs and ionizing radiation (IR).

In both the metastatic and curative settings, a significant number of patients do not benefit from chemotherapy and radiotherapy because their tumors possess robust DNA repair capacity or they develop resistance over time through upregulation of DNA repair pathways. Inhibiting the capacity of cancer cells to repair their DNA following irradiation or treatment with drugs has potential to improve the therapeutic benefit of these treatments, or to reduce the dosage and the adverse effects associated with treatment.

In an aspect of the present application, there is provided compound(s) and composition(s) that inhibit ERCC1-XPF activity. In examples of the present application, the compound(s) and composition(s) inhibit the activity of human ERCC1-XPF activity.

In an aspect of the present application, compound(s) as described herein increase radiosensitivity and/or chemosensitivity of a cell(s) and/or tumour(s).

Compounds as described herein are capable of forming a variety of different salts with various inorganic and organic acids. Such salts are pharmaceutically acceptable for administration to a subject.

In an example of the present application, the compound(s) and composition(s) as described herein increase sensitivity of a cell and/or tumour to radiation.

About half of patients with cancer are treated with radiation therapy, either alone or in combination with other types of cancer treatment. Radiation therapy (also referred to as radiotherapy, X-ray therapy, or irradiation) may be external, internal and systemic. External radiation is delivered from a machine outside the body; internal radiation is implanted into or near the tumour(s); systemic radiation utilizes unsealed radiation sources.

External radiation therapy is used to treat most types of cancer, including but not limited to, cancer of the bladder, brain, breast, cervix, larynx, lung, prostate, and vagina. Intraoperative radiation therapy (IORT) is a form of external radiation that is given during surgery, and can be used to treat localized cancers that cannot be completely removed or that have a high risk of recurring in nearby tissues, including, but not limited to treatment of thyroid and colorectal cancers, gynecological cancers, cancer of the small intestine, and cancer of the pancreas. Prophylactic cranial irradiation (PCI) is another type of external radiation given to the brain when the primary cancer (for example, small cell lung cancer) has a high risk of spreading to the brain.

Internal radiation therapy (or brachytherapy) typically uses radiation source sealed in a small holder called an implant. Implants may be in the form of thin wires, plastic tubes called catheters, ribbons, capsules, or seeds. Interstitial radiation therapy, a type of internal radiation therapy, is inserted into tissue at or near the tumour site. It is used to treat tumors of the head and neck, prostate, cervix, ovary, breast, and perianal and pelvic regions. Intracavitary or intraluminal radiation therapy is inserted into the body with an applicator. It is commonly used in the treatment of uterine cancer, and may have application in other cancers, including breast, bronchial, cervical, gallbladder, oral, rectal, tracheal, uterine, and vaginal.

Systemic radiation therapy uses materials such as iodine 131 and strontium 89, and may be taken by mouth or injected.

In an example of the present application, the radiation is γ-radiation. In another example, the ionizing radiation is X-rays generated by a linear accelerator (Linac).

In another aspect of the present application, there are provided pharmaceutical compositions and methods of treatment using such pharmaceutical compositions for therapeutic uses. In an example of the present application, there is provided a pharmaceutical composition comprising a compound as described herein together with pharmaceutically acceptable diluents or carriers. Suitable pharmaceutical carriers include inert diluents or fillers, water and various organic solvents. The pharmaceutical composition may, if desired, contain additional ingredients such as flavorings, binders, excipients and the like.

In another example, the compound(s) and composition(s) as described herein increases sensitivity of a cell(s) and/or tumour(s) to a chemotherapeutic agent.

In another aspect of the present application, there are provided pharmaceutical compositions and methods of treatment using such pharmaceutical compositions for therapeutic uses. In an example of the present application, there is provided a pharmaceutical composition comprising a compound as described herein together with pharmaceutically acceptable diluents or carriers. Suitable pharmaceutical carriers include inert diluents or fillers, water and various organic solvents. The pharmaceutical composition may, if desired,

Compound(s) of the present application may be administered with a physiologically acceptable carrier. A physiologically acceptable carrier is a formulation to which the compound can be added to dissolve it or otherwise facilitate its administration. Non limiting examples include, but are not limited to, water, saline, physiologically buffered saline.

Solid dosage forms for oral administration can include capsules, tablets, pills, powders, and granules. In such forms, the compound(s) as described herein may be combined with one or more adjuvants, as indicated by the route of administration. Compound(s) as described herein can be admixed with, for example, lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, magnesium or calcium carbonate or bicarbonate. Tablets and pills can additionally be prepared with enteric coatings.

Compound(s) and pharmaceutically acceptable composition(s) as described herein can be administered by parenteral administration, in the form of aqueous or non-aqueous isotonic sterile injection solutions or suspensions. These solutions and suspensions can be prepared from sterile powders or granules having one or more of the carriers or diluents mentioned for use in the formulations for oral administration. Compound(s) as described herein can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art, as know by the skilled worker.

Liquid dosage forms for oral administration include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

It will be appreciated that the amount of active ingredient that can be combined with the carrier materials to produce a single dosage form varies depending upon the mammalian host treated and the particular mode of administration.

Compound(s) and composition(s) as described herein are suitable for combination. Combination therapy as used herein includes administration of the therapeutic agents in a sequential manner, wherein each therapeutic agent is administered at a different time, as well as administration of the therapeutic agents at the same time. As used herein, the therapeutic agents are administered in a sequential manner, wherein each therapeutic agent is administered at a different time, or administered in a generally simultaneous manner. The generally simultaneous administration can be accomplished, for example, by administering to the subject a single capsule having a fixed ratio of each therapeutic agent or in multiple, single capsules for each of the therapeutic agents.

Administration of each therapeutic agent, whether sequential or generally simultaneous, can be effected by any appropriate route including, but not limited to, oral routes, intravenous routes, intramuscular routes, and direct absorption through mucous membrane tissues, etc. The therapeutic agents can be administered by the same route or by different routes.

Combination therapy also includes administration of the therapeutic agents in combination with other biologically active ingredients (such as, but not limited to, a second and different antineoplastic agent) and non-drug therapies (such as, but not limited to, surgery or radiation therapy).

Where the combination therapy further comprises radiation therapy, the radiation therapy may be conducted at a suitable time so long as a beneficial effect from the co-action of the combination of the therapeutic agents and radiation treatment is achieved.

In accordance with another aspect of the present application, there is provided a method(s) for increasing the sensitivity of a cell(s) and/or tumour(s) to chemotherapeutic agents and/or ionizing radiation. In another aspect, there is provided a method(s) for inhibiting ERCC1-XPF activity.

Compound(s) and composition(s) as described herein are chemosensitizers and/or radiosensitizers useful for the treatment of cancer. In an aspect of the present application, the methods, compound(s) and composition(s) of the present invention may be used for the treatment of neoplasia disorders including benign, metastatic and malignant neoplasias. An embodiment of the present application relates to treating or lessening the severity of one or more diseases in which ERCC1-XPF is known to play a role. In an example, the disease is cancer.

In some example, the compound(s) and composition(s) described herein increase radiosensitivity and/or chemosensitivity of a cell(s) and/or tumour(s).

In one aspect, there is provided radiosensitizer and chemosensitizer compounds and compositions, methods and kits and the uses thereof.

The compounds and compositions of the present invention are suitable for combination. Combination therapy as used herein includes administration of the therapeutic agents in a sequential manner, wherein each therapeutic agent is administered at a different time, as well as administration of the therapeutic agents at the same time.

As used herein, the therapeutic agents are administered in a sequential manner, wherein each therapeutic agent is administered at a different time, or administered in a generally simultaneous manner. The generally simultaneous administration can be accomplished, for example, by administering to the subject a single capsule having a fixed ratio of each therapeutic agent or in multiple, single capsules for each of the therapeutic agents.

Administration of each therapeutic agent, whether sequential or generally simultaneous, can be effected by any appropriate route including, but not limited to, oral routes, intravenous routes, intramuscular routes, and direct absorption through mucous membrane tissues, etc. The therapeutic agents can be administered by the same route or by different routes.

Combination therapy also includes administration of the therapeutic agents in combination with other biologically active ingredients (such as, but not limited to, a second and different antineoplastic agent) and non-drug therapies (such as, but not limited to, surgery or radiation therapy).

Where the combination therapy further comprises radiation therapy, the radiation therapy may be conducted at a suitable time so long as a beneficial effect from the co-action of the combination of the therapeutic agents and radiation treatment is achieved.

As used herein, the term “therapeutically effective amount” refers to an amount that is effective for preventing, ameliorating, or treating a disease or disorder (e.g., a infection disease, such as a viral disease).

In an aspect, there is described a compounds, compositions, methods, and uses, for the treatment of a subject having, or suspected of having,

In some examples, the compounds as described herein are an enantiomer, a racemate, a tautomer, or a pharmaceutically acceptable salt, or a solvate, or a functional derivative thereof.

In some examples, there is described a composition comprising a compound as described herein, and a pharmaceutically acceptable carrier, diluent, or vehicle.

A compound or composition may be administered alone or in combination with other treatments, either simultaneously or sequentially, dependent upon the condition to be treated.

In treating a subject, a therapeutically effective amount may be administered to the subject.

Formulations may conveniently be presented in unit dosage form and may be prepared by any methods known in the art. Such methods include the step of bringing the active compound into association with a carrier, which may constitute one or more accessory ingredients. In general, the formulations are prepared by uniformly and intimately bringing into association the active compound with liquid carriers or finely divided solid carriers or both, and then if necessary shaping the product.

The compounds and compositions may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly.

Compounds and/or compositions comprising compounds disclosed herein may be used in the methods described herein in combination with standard treatment regimes, as would be known to the skilled worker.

Methods of the invention are conveniently practiced by providing the compounds and/or compositions used in such method in the form of a kit. Such kit preferably contains the composition. Such a kit preferably contains instructions for the use thereof.

In another example of the present application, there is provided a pharmaceutical composition comprising a compound as described herein, and a pharmaceutically acceptable carrier, diluent, or vehicle.

In another example of the present application, there is provided a use of a compound as described herein, or a composition as described herein for treating a subject having cancer, or suspected of having cancer.

In another example, there is provided a use of a compound as described herein, or a composition as described herein in the manufacture of a medicament for treating a subject having cancer, or suspected of having cancer.

In another example of the present application, there is provided a method of treating a subject having cancer, or suspected of having cancer, comprising, administering a therapeutically effective amount of a compound as described herein, or a composition as described herein.

In another example of the present application, there is provided a compound of as described herein, or a composition as described herein for inhibiting ERCC1-XFP.

In another example, there is provided a compound as described herein, or a composition as described herein for increasing the sensitivity of a cancerous cell of a subject to a chemotherapeutic agent or radiation therapy.

In another example of the present application, there is provided a method of chemosensitizing or radiosensitizing a cancerous cell in a subject in need of chemotherapy or radiation therapy, comprising: administering to said subject a compound as described herein, or a composition as described herein.

In another example of the present application, there is provided a kit comprising: a compound as described herein, or a composition as described herein, and a container, and/or instructions for the use thereof.

To gain a better understanding of the invention described herein, the following examples are set forth. It should be understood that these examples are for illustrative purposes only. Therefore, they should not limit the scope of this invention in anyway.

### EXAMPLES

### Example 1—Targeting DNA Repair in Tumor Cells Via Inhibition of ERCC1-XPF

As described above, preliminary in-vitro assays confirmed that F06 shows promising inhibitory activity and acts synergistically with cisplatin. However, the activity of F06 is suboptimal in terms of clinical properties including potency and safety, and a derivatization strategy, suggested by Jordheim et al (25), was adapted to optimize the action of the compound. Also, previous efforts on developing inhibitors for ERCC1-XPF activity were carried out on the truncated version of the heterodimer, and not the full-length protein (2,25-27). It is reported that the full length ERCC1-XPF protein was 15-fold more active than the truncated form under standard reaction conditions (2,19). Herein described are inhibitors of the activity of the full-length protein. Using F06 as a reference hit, computer-aided drug design techniques were employed such as electrostatic mapping of the F06 binding pocket, molecular docking, pharmacophore modeling and molecular dynamics (MD)-based rescoring to rank F06 derivatives based on their predicted binding affinities to the XPF domain. The reference compound (A1) and top hits (compounds A2-A8) were synthesized and tested for their ability to inhibit the in-vitro endonuclease activity of the full length ERCC1-XPF protein, and the most active compound was further assessed as an inhibitor of the repair of UV-induced thymidine dimers in colorectal cancer cells, as well as a sensitizing agent to not only to UV radiation but also to cyclophosphamide which has been used to significantly reduce proliferation of metastatic colorectal cancer (28).

Results and Discussion

Computer-Aided Drug Design of F06 Analogues

The first step was to investigate the binding mode of the lead compound, F06, to the pocket on the XPF C-terminus, followed by functionalization and extension of the piperazine ring in the F06 compound to: 1) provide potential key interactions via hydrogen bond formation or hydrophobic interactions, and 2) optimize the physicochemical properties and binding affinity for better potency and reduced toxicity of the F06-based compounds.

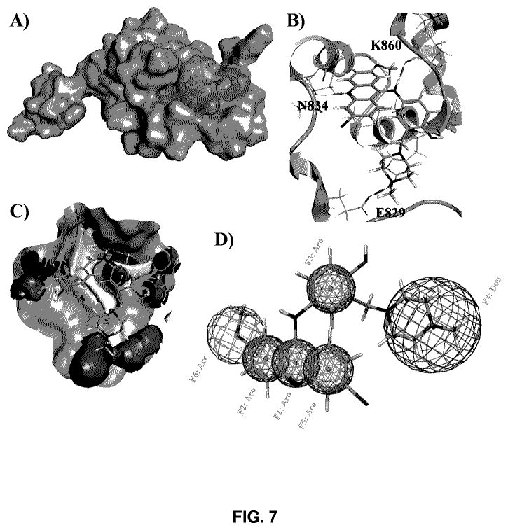

The binding energy for the best docked conformation of the F06 molecule to the XPF structures was −10.23 kcal/mol, as calculated by the Autodock scoring function. The compound showed a high shape complementarity with the XPF pocket, which interacts with the F293 residue of ERCC1 in the dimerized complex (FIG. 7A). Three ligand-receptor hydrogen bonds were observed in the docked pose, namely with the side chain of E829, the backbone of N834 and the backbone of K860. In addition, the hydrophobic core of F06, comprised of three aromatic rings, was positioned within a hydrophobic zone constituted by XPF residues Y833, Q838, M856 and H857 (FIG. 77B). Also, the binding mode of the ligand was in accordance with the spatial distribution of the affinity maps calculated in Molecular Operating Environment 2015 (MOE2015) (FIG. 7C).

To correctly place the scaffold common to all the analogues within the binding site, a pharmacophore model based on the binding pose of F06 was built using MOE. This model included six features, namely four aromatic moieties, one hydrogen bond donor, and one hydrogen bond acceptor features (FIG. 7D and Table 3). Upon generation of such features using MOE, their radii were manually adjusted consistently with the spatial distribution of the affinity maps. The total number of molecular structures that were docked was fifty-seven, accounting for all the different states generated during the preparation step. After the docking was performed, just the top scored pose for each analog was retained. The results were ranked according to their Generalized Born Volume Integral/Weighted Surface Area (GBVI/WSA) score, as calculated by MOE. Then, MD simulations of the complexes were performed, and the analogues rescored using the Molecular Mechanics-Generalized Born Surface Area (MM/GBSA) method. A subset of the top ranked hits was selected for chemical synthesis.

The results of the docking-based VS for this subset are reported in Table 4. The first two hits (compounds 3 and 4) were particularly interesting because of their binding energies in the range of the lead compound, reasonable logP values and conserved ligand efficiencies with respect to the hit structure. The decomposition of the binding energies among the residues of the binding pocket revealed a similar pattern of ligand-receptor interactions for the A3, A4 and F06, although some differences are noticeable for A4.

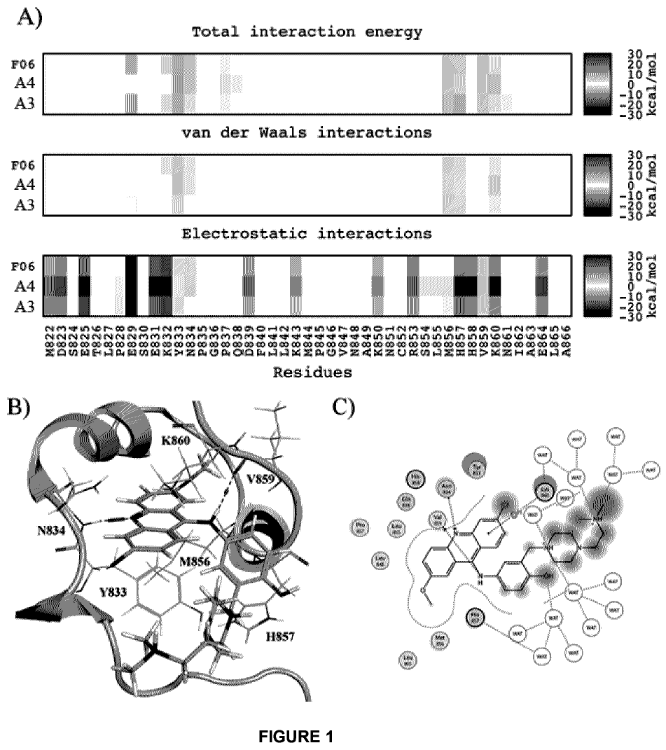

The residues that contribute consistently to the binding of F06 analogues were identified as Y833, M856, H857 and V859, where van der Waals interactions dominated the binding. Electrostatic interactions were divided in highly-favorable with basic residues (D823, E825, E829, E831, D839, E864), and highly-unfavorable with acidic residues (K832, K843, K850, R853, H857, H858, K860), due in part to the positive protonation state of the piperazine ring of the compounds (FIG. 1A). Noteworthy, both contributions were generally higher for A4, when compared with F06 and A3. This may be due to the addition of another positive R-substituent moiety in A4, differently from F06 and A3 where hydrophobic groups were present as R-substituents. A further confirmation of the different nature of the binding was found when analyzing the different contributions of the total binding energy. Indeed, while F06 and A3 showed favorable electrostatic interactions, a highly-unfavorable value was observed for A4. An opposite trend was observed for the polar solvation energy, highly-favorable for A4, and highly-unfavorable for F06 and A3. Van der Waals and non-polar solvation contributions were similar for all the three compounds (Table S3). A detailed analysis of the binding mode of A4 is shown in FIG. 1B. The conserved core structure of the analogue interacted similarly to F06 with the residues constituting the binding site, although the backbone of V859 replaces K860 as hydrogen bond partner during the simulation. In addition, a stable network of water molecules surrounding the binding site and interacting with the exposed R-substituent of A4 was observed during the 2-ns simulation (FIG. 1C). These water-ligand interactions may be responsible for the favorable polar solvation contribution of the binding energy of A4. Charge-charge interactions with the E829 and E831 residues were maintained in terms of average magnitude, when compared with F06.

Synthesis of F06-Based Analogues

Synthesis of compounds A1-A8 was achieved through a one-pot sequential addition reaction in 3 steps as shown in FIG. 6A. Mannich reaction of p-acetamidophenol with formaldehyde and appropriate secondary amine in 2-propanol was carried out under reflux for 12 h. The solvent and excess of the unreacted formaldehyde from the resulting mixture was removed under vacuum and without isolating the compound, the resulting viscous residue was treated with 6 M HCl to deacetylate the acetamido group to furnish the primary amine as depicted in FIG. 6. Afterwards, an equimolar amount of 6,9-dichloro-2-methoxyacridine was added, affording after heating compounds A1-A8 in moderate to good yields after isolation. The sequence is general, facile and reproducible. All synthesized compounds A1-A8 were characterized by ¹H NMR, ¹³C NMR, HRMS, IR and the purity of the most active compound A4 was determined by HPLC (≥98% purity).

According to the in-silico screening, seven compounds were selected to be synthesized among the top hits due to their promising ligand efficiencies and binding energy values in comparison to F06. Derivatization focused primarily on extension of the piperazine ring with different functionalities (i.e alicyclic (A3), substituted aromatics with electron donating (EDG) or electron withdrawing (EWG) groups (compounds A2, A5, A6, and A7), and an aliphatic chain installed with hydrogen bond acceptor (HBA) atom (A4) as shown in Table 2. A8 was synthesized as a control to investigate whether the piperazine ring is crucial for the activity of compounds.

Inhibition of ERCC1-XPF Endonuclease Activity

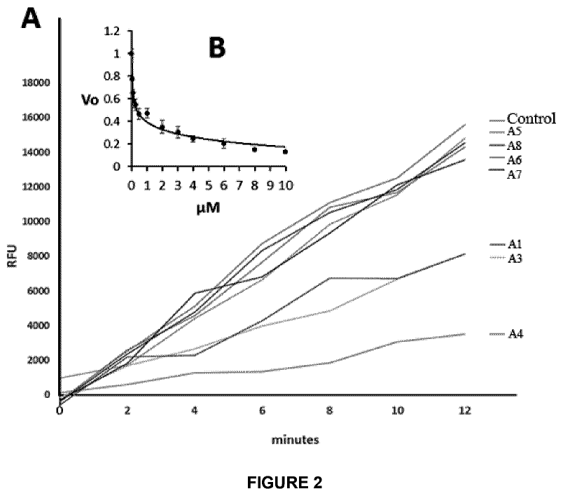

In-vitro real-time fluorescence-based assay was used to assess the inhibitory effect of the synthesized compounds (except A2 due to its instability over time) on ERCC1-XPF endonuclease activity. This assay has been previously described (29), and utilizes a stem-loop substrate (composed of a 10mer-duplex stem and a 20mer-oligodT single-strand loop), labelled on the 5′-terminus with 6-FAM and 3′ with the quencher dabcyl. Upon ERCC1-XPF cleavage in the 10mer-duplex region an 8-base 5′ FAM-labelled product is released resulting in an increased fluorescent signal (FIG. 2A, Control). The other tracings in FIG. 2A indicate to what extent the different compounds (10 μM each) could inhibit scission of the substrate.

As shown herein, three compounds exhibited a marked capacity to inhibit the nuclease activity of ERCC1-XPF, i.e. compounds A1 (F06), A3 and A4. For these three compounds, different concentrations of the drugs were plotted against the initial velocity (Vo, change in relative fluorescence units (RFU)/time) of the enzyme. FIG. 2B (inset) shows an example of data obtained with A4. Half-maximum inhibitory concentrations (IC₅₀) for these compounds were estimated from at least three different experiments for every compound (Table S4).

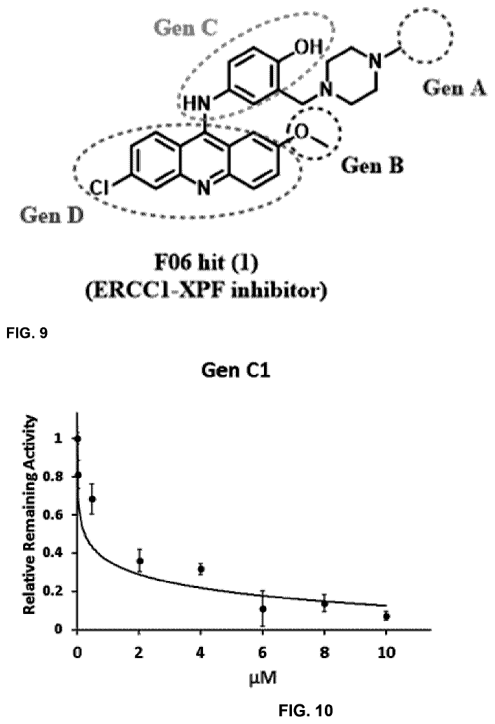

Since acridine compounds are known to bind nucleic acids, the possibility existed that a disruption of the structure of the stem-loop substrate by A4 could have been responsible for the inhibition observed in the ERCC1-XPF kinetic assays. To rule out this possibility the experiment shown in FIG. 9 was carried out. Pre-incubation of ERCC1-XPF with A4 (3 μM) in the reaction medium resulted in a time-dependent loss of enzyme activity. The reactions were started either with addition of the substrate (TO) or with substrate that had been pre-incubated in the reaction medium in the presence of A4 (3 μM) for 12 minutes (T12). The data indicate that the time-dependent inactivation of ERCC1-XPF by A4 was the consequence of the interaction of the drug with the enzyme, and not altered by addition of substrate that had been pre-incubated with the inhibitor. The data in FIG. 9A were obtained with a concentration of A4 approximately 9 times higher than the observed IC₅₀ value of the compound and in the presence of 10% DMSO. When the same test was carried out in the presence of 5% DMSO, a similar time-dependent inhibition was observed and the substrate exhibited comparable behavior (FIG. 9B). (The IC₅₀ values reported in the present study were obtained by incubating ERCCI-XPF in the presence of the indicated concentrations of the drug for 30 minutes in the reaction medium containing 5% DMSO.)

Inhibitor Binding to ERCC1-XPF

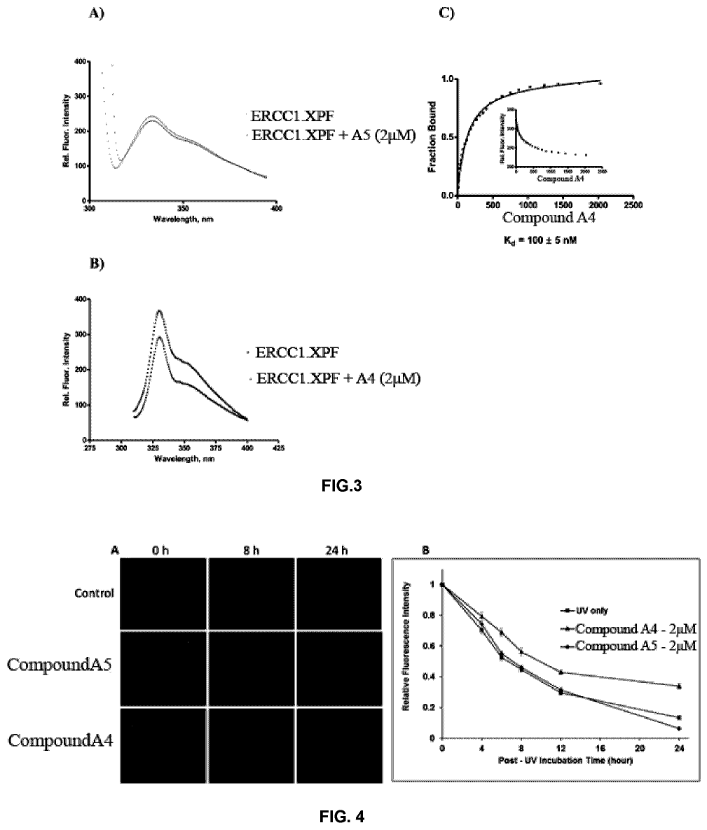

Intrinsic fluorescence spectroscopy (of the protein tryptophan residues) was utilized to study the binding affinity of A4 (active compound) and A5 (non-active compound) to ERCC1-XPF (FIG. 3). Addition of 2 μM of A5 had no significant effect on protein fluorescence and the observed fluorescence quenching at 330 nm was only 3±1%, thus providing no evidence of any interaction. In contrast, addition of A4 (2 μM) induced nearly 23±2% quenching of protein fluorescence at 330 nm, clearly indicating interaction of A4 with the ERCC1-XPF complex as depicted in FIG. 3B. Binding affinity (in terms of dissociation constant, K_{d}) of A4 for ERCC1-XPF complex was determined by following fluorescence quenching (a measure of ligand binding) as a function of ligand concentration. A representative plot of relative fluorescence intensities versus the concentration of A4 is shown in FIG. 3C (inset). Nonlinear regression analysis (GraphPad Prism Software, San Diego, Calif.) of the binding data was carried out as described in our earlier paper (41), and revealed a unimodal binding with a K_{d} value of 100±5 nM as also shown in Table 6.

Inhibition of Cellular Repair of Cyclobutane Pyrimidine Dimers

Significant inhibition of NER determined by the removal of UV-induced CPDs compared with control cells over 24 hours is depicted in FIG. 4.

Immunofluorescent detection of CPDs after the exposure of HCT116 colorectal cells to 8 J/m² treated with A4 showed a significant inhibition of removal of CPDs compared with control cells over 24 hours. Based on these results and others (30), approximately 80% of CPDs are removed over 24 hours after UV exposure of HCT116 cells, but this was reduced to approximately 60% in the presence of A4. On the other hand, A5, which demonstrated very limited inhibition in vitro, failed to inhibit the cellular removal of CPDs. FIG. 4B shows the relative quantification of CPDs after treatment with active (A4) and inactive (A5) compounds.

Increased Sensitization to UV Radiation and Cyclophosphamide

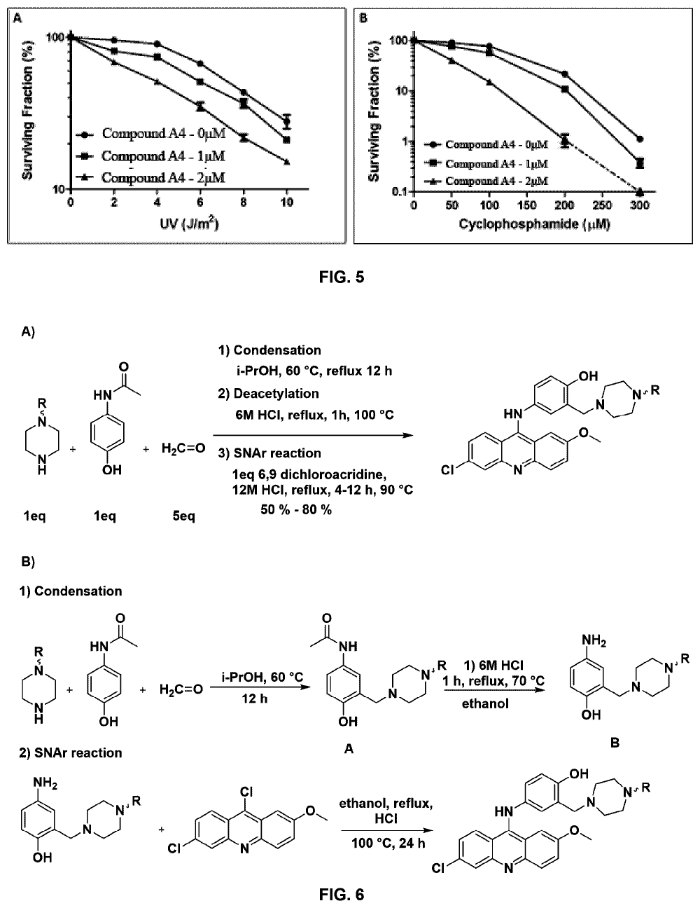

Also evaluated was the effectiveness of A4 (1 and 2 μM) to sensitize cells to UV irradiation based on clonogenic survival as shown in FIG. 5A. In accord with the repair data, 2 μM A4 significantly reduced survival of the UV irradiated cells.

Then examined was the combined effect of A4 (1 and 2 μM) on cellular survival following exposure to the DNA interstrand crosslinking agent cyclophosphamide. The survival curves shown in FIG. 5B indicate that, at a concentration of 2 μM, A4 significantly sensitized the cells to cyclophosphamide (starting at 50 μM) and no colonies were detected on the plates with a cyclophosphamide concentration of 300 μM.

### Conclusion

The ERCC1-XPF heterodimer is a structure-specific endonuclease, which is required especially for NER and ICL DNA repair pathways. Although its action is essential to maintain genome integrity and to protect against damage-induced mutations, as part of the NER and ICL machinery it can counteract the effect of DNA damaging therapies such as platinum-based chemotherapy and radiotherapy. A promising approach to enhance the effect of such therapies is to inhibit the action of DNA repair in cancer cells using small molecules. In this work, a computational drug design workflow was used to provide a rational design for novel analogues of the F06 molecule (A1), a lead inhibitor targeting the dimerization between XPF and ERCC1, which is required for endonuclease activity. Identified were seven compounds for which in-silico simulations predicted attractive properties, namely binding affinities and ligand efficiency towards an XPF site on the dimerization interface. The synthesis of the computationally designed compounds was successfully carried out using a simple, robust and reproducible synthetic strategy. Interestingly, A4 has good physicochemical properties, i.e. reasonable logP and ligand efficiency values, and small molecular weight (see Table 4), and thus is a potential lead candidate for further optimization. Following structure-activity relationship studies and in-vitro screening, this approach yielded compounds A3 and A4 as potent inhibitors of ERCC1-XPF activity. An in-vitro ERCC1-XPF endonuclease assay identified A4 as the best ERCC1-XPF inhibitor with an IC₅₀ value of 0.33 μM compared to 1.86 μM for 1. In addition, the K_{d} value for this compound was experimentally measured as 100 nM. A4 also showed a significant inhibition of the removal of cyclobutane pyrimidine dimers compared with control cells after exposure of HCT116 cells to UV radiation and sensitized the cells to UV and cyclophosphamide-induced cytotoxicity, indicating inhibition of NER and ICL repair. Computational workflow successfully identified superior compounds from a set of analogues differing by one substituent group. Additionally, it provided detailed information about the ligand-protein interaction. A4 was the best in vitro analogue. Detailed analysis of the MM/GBSA binding energies, together with the visual analysis of the simulation results, suggests a binding mode for A4 where the conserved hydrophobic core of the analogue is buried inside the XPF binding site and, differently to the other active compounds (1 and 3) the positive R-substituent is favorably exposed to the solvent. Although all of the compounds share the acridine moiety, the assay showed that the inhibition activity of A4 was the consequence of the interaction of the drug with the enzyme, and not the result of binding with DNA. The fact that compounds A4 and A5 (negative control) show drastically different activity despite possessing the same acridine moiety also provides strong evidence that inhibition is not mediated via DNA intercalation.

Methods

Molecular Docking of F06 (1)

First step of the VS study was to investigate the binding mode of the lead compound A1 (25) to the pocket on the XPF C-terminus. Used was twenty XPF structures as single targets for a relaxed complex scheme (RCS) docking protocol, in order to accurately account for protein flexibility (31,32). These structures were extracted from the Protein Data Bank (PDB)(33) entry 1Z00 (17), reporting the NMR ensemble of the dimerization complex between the ERCC1 and XPF HhH2 domains. The XPF structures were optimized with a minimization process, details of which are reported in Example 2—Supporting Information, section 1.1. The binding site of each target was defined as the geometric center of the residues Y833, N834, P837, Q838, M856, K860, N861 and 1862 on the XPF HhH2 domain, as reported in our previous study (25), where the residues are numbered according to the 1Z00 PDB file. Molecular docking simulations were performed using the Lamarckian Genetic Algorithm (LGA) (34) and the built-in scoring function (35) of Autodock4 (36). Technical details regarding the docking simulations are reported in Example 2—Supporting Information, section 1.2.

Characterization of the Binding Pocket and Pharmacophore Modeling

To characterize the binding site and the binding mode of A1, electrostatic maps were generated in the MOE2015 software package (37), based on the docked pose of A1, using −2 kcal/mol for the potential isosurfaces of hydrophobic, acceptor and donor probe atoms, and the Poisson-Boltzmann equation to compute the potentials. The maps were built within 4.5 Å of the ligand pose obtained from docking. Using the information from the docking and electrostatic mapping, a pharmacophore model for A1 was built with the MOE Pharmacophore Query Editor and the EHT scheme (38).

Docking-Based Virtual Screening of F06 Analogues

Fifty-seven analogues of the F06 structure were designed by replacement of the piperazine N-methyl group with other moieties expected to capture additional binding interactions of the piperazine ring. The structures were prepared using Schrödinger LigPrep (39) to account for multiple tautomers, protonation states and low-energy ring conformations, using the same approach as for A1 (Example 2—Supporting Information, section 1.2). The molecular docking simulations were performed with MOE Dock. Only the XPF structure from the best F06 complex was used as a target for the VS study. Different conformations of the analogues were obtained using Conformation Import. The previously built pharmacophore model was used for the placement step, in which 30 poses are returned according to the London dG scoring method (40). To account for the local arrangement of the pocket residues upon ligand binding, the Induced Fit method was selected for the refinement step, where the side chains of the binding pocket were left to move freely. At the end, one pose scored with the Generalized Born Volume Integral/Weighted Surface Area (GBVI/WSA) function was returned (40). Water/octanol partition coefficients (logP) of the molecules were calculated in MOE using the SlogP function (41), which takes into account the given protonation state of the molecule under examination. Ligand efficiencies were calculated as the ratio between the computed binding energies and the number of heavy atoms of each analog.

Molecular Dynamics Simulations and MM/GBSA Rescoring of the Analogues

To calculate an average binding energy for the hits, performed were 2 ns of MD simulations for the top ranked compound-XPF complexes. Details of the preparation method and the parameters used in these simulations are reported in Example 2—Supporting Information, section 2.1. Free energy calculations were performed over the trajectories with the MM/GBSA method, using the MMPBSA.py script (42). For technical details regarding the calculations refer to Example 2—Supporting Information, section 2.1. The calculations were performed on snapshots extracted every 10 ps from the MD trajectories, and per-residue decompositions of the binding energies were also performed for the residues within 10 Å of any analog atom at the beginning of the simulations. Also calculated were the entropic contribution of ligand binding using the normal mode analysis (NMA) method (43). The final binding energies used to rank the compounds were calculated combining the MM/GBSA and entropy contributions with the equation S1 in Example 2—Supporting Information.

General Experimental Procedures for Preparation of the Top F06-Based Analogues

Synthesis and characterization of compounds A1-A8 are described in Example 2—Supporting Information, section 6. Reactions were carried out in flame-dried glassware under a positive argon atmosphere unless otherwise stated. Transfer of anhydrous solvents and reagents was accomplished with oven-dried syringes or cannulae. Solvents were distilled before use. Chemicals were purchased from Sigma Aldrich Inc., and were used without further purification. Thin layer chromatography was performed on glass plates pre-coated with 0.25 mm silica gel. Flash chromatography columns were packed with 230-400 mesh silica gel. Proton nuclear magnetic resonance spectra (¹H NMR) were recorded at 500 MHz, and coupling constants (J) are reported in hertz (Hz). Standard notation was used to describe the multiplicity of signals observed in ¹H NMR spectra: broad (br), multiplet (m), singlet (s), doublet (d), triplet (t), etc. Carbon nuclear magnetic resonance spectra (¹³C NMR) were recorded at 125 MHz and are reported (ppm) relative to the center line of the triplet from chloroform-d (77.0 ppm) or the center line of the heptuplet from methanol-d4 (49.0 ppm). Infrared (IR) spectra were measured with a FT-IR 3000 spectrophotometer. Mass spectra were determined on a high-resolution electrospray positive ion mode spectrometer. The high-performance liquid chromatography (HPLC) analyses were performed using an Agilent 1100 LC/MSD instrument. Elution was done with a gradient of 10-95% solvent B in solvent A (solvent A was 0.1% TFA in water, and solvent B was 0.1% acetic acid in MeCN) through an Agilent column eclipse XDB-C18 (4.6×250 mm, 5 μm) column at 1.0 mL/min. Area % purity was measured at 210 and 254 nm. The purity of the most active compound was assessed by HPLC (>95%).

ERCC1-XPF Protein Preparation

Human ERCC1-XPF wild-type protein was obtained as previously described (29). Basically, the recombinant protein was expressed from a bicistronic plasmid (provided by Dr. Richard Wood, University of Texas MD Anderson Cancer Center, Smithville, Tex.) in the E. coli BL21 (DE3) strain. Since both XPF and ERCC1 contained a polyhistidine (His-6) tag, the proteins extracted from E. coli were incubated with a ProBond Nickel-Chelating Resin (Thermo Fisher Scientific). Protein eluted from the Ni affinity column was subsequently loaded into a Hi-trap heparin column (GE Healthcare). Fractions recovered from the heparin column that contained ERCC1-XPF were dialyzed, concentrated and stored at −80° C. in 10 mM HEPES pH 7.4, 2.5 mM p-mercaptoethanol, 0.01% CHAPS, 0.25 mM EDTA, 50% glycerol and 25 mM NaCl.

Microplate Fluorescence Incision Assay

A previously described protocol (2,29) was followed. Briefly, reactions were carried out in 384-well black, flat-bottomed microtiter plates (OptiPlate—384 F, Perkin Elmer) in a total volume of 20 μl containing the indicated concentrations of inhibitor compounds, 100 nM stem-loop substrate [6-FAM-5′-CAGCGCTCGG(20T)CCGAGCGCTG-3′-dabcyl] (SEQ ID NO: 1), 25 ng ERCC1-XPF, 50 mM Tris-CI pH 8, 20 mM NaCl, 0.5 mM DDT and 0.75 mM MnCl₂ at 25° C. Fluorescent readings were obtained using a FLUOstar Optima fluorimeter (BMG Labtech) with Optima software at an excitation of and emission wavelengths of 485 and 520 nm respectively, for 12 minutes.

Steady-State Fluorescence Assays

Steady-state fluorescence spectra were measured at room temperature on a Perkin-Elmer LS-55 spectrofluorometer (Freemont, Calif.) with 5 nm spectral resolution for excitation and emission using 30-80 nM solution of purified recombinant ERCC1-XPF protein complex. Protein fluorescence was excited at 295 nm, and fluorescence emission spectra were recorded in the 300-400 nm range: changes in fluorescence intensity was monitored at the emission maximum (330 nm). In studying the effects of inhibitors on protein fluorescence intensities, additions to protein samples were made from inhibitors stock solutions, keeping the protein dilution below 3%.

Cell Culture

Human colorectal cancer HCT116 cell line was obtained from the American Type Culture Collection (ATCC). The cell population was expanded immediately after arrival, aliquoted and stored frozen in liquid nitrogen. Freshly thawed cells were used for each experiment. The cells were cultured in a 1:1 DMEM/F12 media supplemented with 10% FBS, 50 units/mL penicillin, 50 μg/mL streptomycin, mM 1-glutamine, 0.1 mM nonessential amino acids, 1 mM sodium pyruvate and maintained under 5% CO₂ in a humidifier incubator at 37° C. All the supplies for cell culture were obtained from Gibco/BRL.

Cellular Repair of Cyclobutane Pyrimidine Dimers

Protocol of Mirzayans et al. was followed with minor modifications (30). Approximately 1×10⁵ HCT116 cells were seeded on each coverslip and allowed to attach overnight. Medium was then removed and cells were treated for one hour with the desired compound. The medium was removed, and cells were exposed to 8 J/m² UV-C radiation (G15T8 254 nm lamp, Ushio America Inc, Cypress, Calif.) followed by adding fresh medium containing the compound. Plates were incubated at 37° C. for different periods of time up to 24 hours and fixed in 50:50 methanol/1× phosphate-buffered saline (PBS) solution, followed by replacing the methanol/PBS solution with 100% methanol and incubation in −20° C. After 20 minutes, methanol was removed, and cells were treated with PBS for 5 minutes in room temperature. After fixing, cells were permeabilized in 0.5% Triton/PBS and washed with PBS, denatured in 2 N HCl, and neutralized by twice washing with 0.1 M borate buffer pH 8.5. Cells on cover slips were washed once with PBS followed by blocking with 5% non-fat dry milk/PBS for 30 minutes. Mouse anti-thymine dimer monoclonal antibody (Cat. No. MC-062, Kamiya Biomedical Company, Seattle, Wash.) was applied to the cover slips for one hour in the dark and at room temperature. Cells were then washed with PBS containing 0.1% Tween-20 and incubated with rabbit anti-mouse IgG-Alexafluor antibody (Invitrogen, Carlsbad, Calif.) followed by two washes with PBS/(0.1%)Tween-20. Cells on coverslips were then washed with PBS, rinsed with water, and mounted on slides using DAPI glycerol mounting solution. Slides were kept at 4° C. before fluorescent microscopic evaluation and measurement of fluorescence intensity with MetaXpress Version: 6.2.1.704 software (Molecular Devices, Sunnyvale, Calif.).

Clonogenic Survival Assay

UV treatment: HCT116 cells (100-800 cells depending on the UV dose) were plated in triplicate in 60-mm petri dishes. The cells were incubated overnight at 37° C. in a humidified atmosphere containing 5% CO₂ to allow for cell attachment. Medium was then removed, and cells were treated with 1 or 2 μM A4 for one hour. Medium was removed again, and cells were exposed to increasing doses (0-10 J/m²) of UV-C radiation and then incubated for a further 10 days in the presence of inhibitor at 37° C. in a 5% CO₂ atmosphere to allow for colony formation. After this period plates were stained with crystal violet and colonies were counted using a Colcount instrument (Oxford Optronix, Abingdon UK). Finally, plating efficiency and surviving fraction were calculated.

Cyclophosphamide treatment: A similar protocol was followed as described for the UV treatment, except that cells were treated with 1 or 2 μM A4 for 4 hours followed by addition of increasing doses of cyclophosphamide (0-300 μM). After 24 hours medium was replaced with fresh medium containing A4 alone. Plates were incubated for another 8 days at 37° C. in a 5% CO₂ atmosphere for colony formation. After this period plates were stained with crystal violet, colonies were counted, and plating efficiency and surviving fraction were calculated.

### REFERENCES

1. Sijbers A M, de Laat W L, Ariza R R, Biggerstaff M, Wei Y-F, Moggs J G, et al. Xeroderma pigmentosum group F caused by a defect in a structure-specific DNA repair endonuclease. Cell 1996; 86(5):811-22.
2. McNeil E M, Astell K R, Ritchie A-M, Shave S, Houston D R, Bakrania P, et al. Inhibition of the ERCC1-XPF structure-specific endonuclease to overcome cancer chemoresistance. DNA Repair 2015; 31:19-28.
3. Friedberg E C, Walker G C, Siede W, Wood R D, Schultz R A, Ellenberger T. DNA Repair and Mutagenesis. 2nd edn ASM Press. Washington, DC 2006.
4. McNeil E M, Melton D W. DNA repair endonuclease ERCC1-XPF as a novel therapeutic target to overcome chemoresistance in cancer therapy. Nucleic Acids Research 2012; 40(20):9990-10004.
5. Douwel D K, Hoogenboom W S, Boonen RACM, Knipscheer P. Recruitment and positioning determine the specific role of the XPF-ERCC1 endonuclease in interstrand crosslink repair. The EMBO Journal 2017:e201695223.
6. Yagi T, Katsuya A, Koyano A, Takebe H. Sensitivity of group F xeroderma pigmentosum cells to UV and mitomycin C relative to levels of XPF and ERCC1 overexpression. Mutagenesis 1998; 13(6):595-9.
7. Gajjar K K, Yadav D K, Kobawala T P, Trivedi T I, Vora H H, Ghosh N R. ERCC1 expression in patients with colorectal cancer: a pilot study. Journal of Cancer Metastasis and Treatment; Vol 2, No 12 (2016) 2016.
8. McHugh P J, Spanswick V J, Hartley J A. Repair of DNA interstrand crosslinks: molecular mechanisms and clinical relevance. The Lancet Oncology 2001; 2(8):483-90.
9. Ahmad A, Robinson A R, Duensing A, van Drunen E, Beverloo H B, Weisberg D B, et al. ERCC1-XPF endonuclease facilitates DNA double-strand break repair. Molecular and Cellular Biology 2008; 28(16):5082-92.
10. de Laat W L, Appeldoorn E, Jaspers N G J, Hoeijmakers J H J. DNA structural elements required for ERCC1-XPF endonuclease activity. Journal of Biological Chemistry 1998; 273(14):7835-42.
11. Gaillard P-HL, Wood R D. Activity of individual ERCC1 and XPF subunits in DNA nucleotide excision repair. Nucleic Acids Research 2001; 29(4):872-9.
12. Gentile F, Tuszynski J A, Barakat K H. New design of nucleotide excision repair (NER) inhibitors for combination cancer therapy. Journal of Molecular Graphics and Modelling 2016; 65:71-82.
13. Gentile F, Barakat K H, Tuszynski J A. Computational Characterization of Small Molecules Binding to the Human XPF Active Site and Virtual Screening to Identify Potential New DNA Repair Inhibitors Targeting the ERCC1-XPF Endonuclease. International Journal of Molecular Sciences 2018; 19 (5).
14. Gentile F, A Tuszynski J, H Barakat K. Modelling DNA repair pathways: recent advances and future directions. Current Pharmaceutical Design 2016; 22(23):3527-46.
15. Ahmad A, Enzlin J H, Bhagwat N R, Wijgers N, Raams A, Appledoorn E, et al. Mislocalization of XPF-ERCC1 nuclease contributes to reduced DNA repair in XP-F patients. PLoS Genetics 2010; 6 (3):e1000871.
16. de Laat W L, Sijbers A M, Odijk H, Jaspers N G J, Hoeijmakers J H J. Mapping of interaction domains between human repair proteins ERCC1 and XPF. Nucleic Acids Research 1998; 26(18):4146-52.
17. Tripsianes K, Folkers G, Eiso A B, Das D, Odijk H, Jaspers N G J, et al. The structure of the human ERCC1/XPF interaction domains reveals a complementary role for the two proteins in nucleotide excision repair. Structure 2005; 13(12):1849-58.
18. Choi Y-J, Ryu K-S, Ko Y-M, Chae Y-K, Pelton J G, Wemmer D E, et al. Biophysical characterization of the interaction domains and mapping of the contact residues in the XPF-ERCC1 complex. Journal of Biological Chemistry 2005; 280(31):28644-52.
19. Tsodikov O V, Enzlin J H, Scharer O D, Ellenberger T. Crystal structure and DNA binding functions of ERCC1, a subunit of the DNA structure-specific endonuclease XPF-ERCC1. Proceedings of the National Academy of Sciences of the United States of America 2005; 102(32):11236-41.
20. McNeil E M, Astell K R, Ritchie A-M, Shave S, Houston D R, Bakrania P, et al. Inhibition of the ERCC1-XPF structure-specific endonuclease to overcome cancer chemoresistance. DNA Repair 2015; 31:19-28.
21. Chapman T M, Gillen K J, Wallace C, Lee M T, Bakrania P, Khurana P, et al. Catechols and 3-hydroxypyridones as inhibitors of the DNA repair complex ERCC1-XPF. Bioorganic & Medicinal Chemistry Letters 2015; 25(19):4097-103.
22. Chapman T M, Wallace C, Gillen K J, Bakrania P, Khurana P, Coombs P J, et al. N-Hydroxyimides and hydroxypyrimidinones as inhibitors of the DNA repair complex ERCC1-XPF. Bioorganic & Medicinal Chemistry Letters 2015; 25(19):4104-8.
23. Arora S, Heyza J, Zhang H, Kalman-Maltese V, Tillison K, Floyd A M, et al. Identification of small molecule inhibitors of ERCC1-XPF that inhibit DNA repair and potentiate cisplatin efficacy in cancer cells. Oncotarget 2016; 7(46):75104-17.
24. Yang L, Ritchie A-M, Melton D W. Disruption of DNA repair in cancer cells by ubiquitination of a destabilising dimerization domain of nucleotide excision repair protein ERCC1. Oncotarget 2017; 8(33):55246-64.
25. Jordheim L P, Barakat K H, Heinrich-Balard L, Matera E-L, Cros-Perrial E, Bouledrak K, et al. Small molecule inhibitors of ERCC1-XPF protein-protein interaction synergize alkylating agents in cancer cells. Molecular Pharmacology 2013; 84(1):12-24.
26. Chapman T M, Gillen K J, Wallace C, Lee M T, Bakrania P, Khurana P, et al. Catechols and 3-hydroxypyridones as inhibitors of the DNA repair complex ERCC1-XPF. Bioorganic & Medicinal Chemistry Letters 2015; 25(19):4097-103.
27. Arora S, Heyza J, Zhang H, Kalman-Maltese V, Tillison K, Floyd A M, et al. Identification of small molecule inhibitors of ERCC1-XPF that inhibit DNA repair and potentiate cisplatin efficacy in cancer cells. Oncotarget 2016; 7(46):75104-17.
28. Scurr M, Pembroke T, Bloom A, Roberts D, Thomson A, Smart K, et al. Low-Dose Cyclophosphamide Induces Antitumor T-Cell Responses, which Associate with Survival in Metastatic Colorectal Cancer. Clinical cancer research: an official journal of the American Association for Cancer Research 2017; 23(22):6771-80.
29. Bowles M, Lally J, Fadden A J, Mouilleron S, Hammonds T, McDonald N Q. Fluorescence-based incision assay for human XPF-ERCC1 activity identifies important elements of DNA junction recognition. Nucleic Acids Research 2012; 40 (13):el01-e.
30. Mirzayans R, Pollock S, Scott A, Gao C Q, Murray D. Metabolic labeling of human cells with tritiated nucleosides results in activation of the ATM-dependent p53 signaling pathway and acceleration of DNA repair. Oncogene 2003; 22(36):5562-71.
31. Philippopoulos M, Lim C. Exploring the dynamic information content of a protein NMR structure: Comparison of a molecular dynamics simulation with the NMR and X-ray structures of Escherichia coli ribonuclease HI. Proteins: Structure, Function, and Bioinformatics 1999; 36(1):87-110.
32. Ishima R, Torchia D A. Protein dynamics from NMR. Nature Structural & Molecular Biology 2000; 7(9):740.
33. Berman H M, Westbrook J, Feng Z, Gilliland G, Bhat T N, Weissig H, et al. The Protein Data Bank. Nucleic Acids Research 2000; 28(1):235-242.
34. Morris G M, Goodsell D S, Halliday R S, Huey R, Hart W E, Belew R K, et al. Automated docking using a Lamarckian genetic algorithm and an empirical binding free energy function. Journal of Computational Chemistry 1998; 19(14):1639-62.
35. Huey R, Morris G M, Olson A J, Goodsell D S. A semiempirical free energy force field with charge-based desolvation. Journal of Computational Chemistry 2007; 28(6):1145-52.
36. Morris G M, Huey R, Lindstrom W, Sanner M F, Belew R K, Goodsell D S, et al. AutoDock4 and AutoDockTools4: Automated docking with selective receptor flexibility. Journal of Computational Chemistry 2009; 30(16):2785-91.
37. Chemical Computing Group I. Molecular Operating Environment (MOE). 2013.082017.
38. Lin A. Overview of Pharmacophore Applications in MOE. 2004.
39. Schrödinger. LigPrep. 2013.
40. Labute P. The generalized Born/volume integral implicit solvent model: estimation of the free energy of hydration using London dispersion instead of atomic surface area. Journal of Computational Chemistry 2008; 29(10):1693-8.
41. Wildman S A, Crippen G M. Prediction of physicochemical parameters by atomic contributions. Journal of Chemical Information and Computer Sciences 1999; 39(5):868-73.
42. Miller lii BR, McGee Jr T D, Swails J M, Homeyer N, Gohlke H, Roitberg A E. MMPBSA. py: an efficient program for end-state free energy calculations. Journal of Chemical Theory and Computation 2012; 8(9):3314-21.
43. Kassem S, Ahmed M, EI-Sheikh S, Barakat K H. Entropy in bimolecular simulations: A comprehensive review of atomic fluctuations-based methods. Journal of Molecular Graphics and Modelling 2015; 62:105-17.

### Example 2—Supporting Information, Targeting DNA Repair in Turner Cells Via Inhibition of ERCC1-XPF

Methods

1. Molecular Docking of F06

1.1 Target Preparation

Twenty NMR structures of the ERCC1-XPF complex (PDB entry 1Z00) were prepared. Initially the ERCC1 terminus was removed, as the previous study identified the best suitable binding pocket being on the XPF dimerization surface (1). Then assigned was the protonation states of the XPF residues the H++ server (2) using pH 7, a salinity of 0.15 M, a protein and solvent dielectric constants of 10 and 80, respectively. In AmberTools14 tleap (3), Amber ff14SB force field parameters were assigned to the protein (4), whereas the Li, Song and Merz's 12-6-4 parameters for mono and divalent ions in TIP3P water have been used for the ions (5,6). The protein was solvated with an octahedral box of TIP3P explicit water molecules with 15 Å of buffer (minimal distance between any protein atom and the edge of the box). Na+ and Cl− ions have been added to the system to neutralize it and reach a 0.15 M physiological ionic concentration. Then performed was a two steps minimization in Amber pmemd.cuda (7) with the following procedure: 1000 steps of conjugate gradients method keeping harmonically restrained the whole protein (force constant of 500 kcal/mol/Å²) to relax ion and water positions; followed by 1000 steps of conjugate gradients method keeping restrained the backbone atoms with a force constant of 2 kcal/mol/Å². A cutoff of 9 Å was used during this relaxation process.

1.2 Docking Simulations

The 2D structure of F06 was downloaded from the PubChem repository (8) (entry 421105). Different protonation states were obtained using Epik, tautomers and up to three low energy ring conformations at pH 7.0+−2.0 of the compound have been calculated in Schrödinger LigPrep, using the OPLS-2005 force field (9). The docking box for each XPF structure was defined as 56X50X56 points spaced by 0.375 Å and centered in the geometric center of the residues Tyr833, Asn834, Pro837, Gln838, Met856, Lys860, Asn861 and Ile862 (1), as calculated in Visual Molecular Dynamics (VMD) (10). Affinity, electrostatic and desolvation maps were obtained using Autogrid4 (11). The Lamarckian genetic algorithm (LGA) of Autodock4 (12) was used for the docking simulations, with a population size of 300, 25000000 maximal energetic evaluations, 27000 maximal generations and one survival for each generation with the rates of mutation and crossover set to 0.02 and 0.8, respectively. For each simulation, 100 LGA runs were performed and the results were clustered based on the root-mean squared deviation (RMSD) metric using 2 Å as tolerance; just the simulations resulting in at least a cluster with more than 25 conformations were considered. The Autodock scoring function (13) was used to rank the results. Hydrogen bonds were detected in VMD with the following parameters: distance cutoff of 3.5 Å and angle cutoff of 120° (60° of deviation from linearity).

2. MD Simulations and MM/GBSA Rescoring of the Analogues

2.1 Molecular Dynamics Simulations of Top Hit Complexes

Antechamber (14) was used to assign the General Amber Force Field (GAFF) (15) parameters to the compounds. The ff14SB force field parameters were used for the protein. The same explicit solvation and ionic concentration setup introduced previously was employed. Molecular dynamics (MD) simulations of the systems were performed on pmemd.cuda using the following procedure: 1) relaxation of ions and water molecule positions using 1000 steps of steepest descent and 1000 steps of conjugate gradients minimization, keeping all the complex atoms fixed through an harmonic restraint (force constant of 500 kcal/mol/Å²). 2) 2000 steps of steepest descent followed by 3000 steps of conjugate gradients methods without restraints. 3) Gradual heating of the system from 0 to 300 K in 100 ps using Langevin dynamics, keeping restrained the backbone atoms (2 kcal/mol/Å²) and using a time step of 0.5 fs and NVT conditions. 4) Gradual release of the restraint in four phases of 50 ps each at constant pressure (1 atm). The time step was set to 2 fs. 5) 2 ns of production simulation, recording the coordinates every 2 ps, similar to what was done in our previous work (16).

The enthalpic contribution to the change in free energy due to the binding of the ligands has been estimated using the MM/GBSA method using the script MMPBSA.py (17); the change of the energy of a complex configuration is calculated as

ΔG_{bind,solv}=ΔG_{MM,vac}+ΔG_{solv,complex}−(ΔG_{solv,ligand}+ΔG_{solv,protein})−TΔS (Eq. S1)

where ΔG_{MM,vac} is calculated as the sum of electrostatic and van der Waals interactions occurring between the protein and the ligand. TAS is the term modeling the change in conformational entropy due to the binding. The solvation terms are modeled as

ΔGₛₒₗᵥ=ΔG_{solv,polar}+ΔG_{solv,npolar} (Eq. S2)

where the polar contribution of the solvent is calculated solving the Generalized Born equation (18). The ionic concentration was set to 0.15 M. igb flag was set to 5 (19) and mbondi2 radii were used. The hydrophobic contribution to the solvation free energy has been calculated as

ΔG_{solv,npolar}=γ·SASA (Eq. S3)

where γ (surface tension) was 0.005 kcal/mol/Å2 and A the solvent accessible surface area (SASA) calculated using the linear combinations of pairwise overlaps (LCPO) model method (20).

3. Synthesis of F06-Based Analogues

See FIG. 6, which depicts A) a one-pot sequential addition reaction for synthesis of compounds A1-A8, and B) main steps and intermediates included in the one pot sequential addition reaction.

4. Cell Proliferation Assay

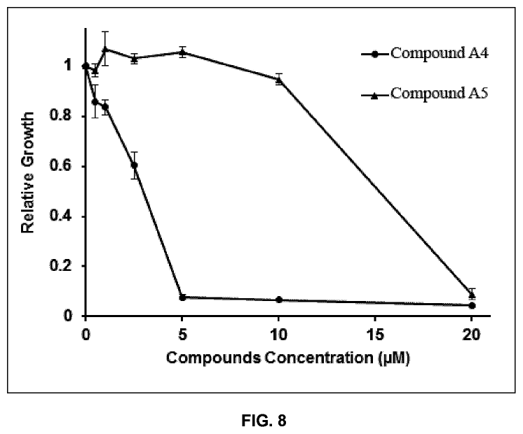

The Cell Titer 96@ Aqueous One Solution Cell Proliferation Assay (MTS) (Promega, Madison, Wis.) was used to measure the proliferation of HCT116 colorectal cancer cells. Briefly, 3,000 cells were seeded in 100 μl of culture medium. After 24 hours, the medium was aspirated and new culture medium without (control) and with inhibitory compounds were added to each well. After 72 hours 20 μl of MTS [3-(4, 5-dimethylthiazol-2-yl)-5-(3-carboxymethoxyphenyl)-2-(4-sulfophenyl)-2H-tetrazolium] solution was added to each well and plates were incubated for 2 hours at 37° C. in a humidified atmosphere containing 5% CO₂ followed by reading the absorbance of treated and untreated cells at 490 nm wavelength with a microplate reader, and plotting the corrected absorbance at 490 nm versus concentration of drug as shown in FIG. 8.

Results

5. Virtual Screening of F06 Analogues

Please see FIG. 7, a visual analysis of the F06 docking pose. Also see Tables S1-S4.

6. Synthesis and Characterization of Inhibitors (A1-A8)

### 4-((6-Chloro-2-methoxyacridin-9-yl)amino)-2-((4-methylpiperazin-1-yl)methyl)phenol (A1)

Synthesis of A1 was achieved through one pot sequential addition reaction in 3 steps. A mixture of N-methyl piperazine (0.12 mL, 1.0 mmol), acetaminophen (0.15 g, 1.0 mmol) and 37% formaldehyde (0.10 mL, 5 mmol) in isopropyl alcohol (2 ml) was taken in 10 mL single neck round bottom flask. Then, the reaction mixture was stirred and heated under reflux at 65° C. for 6 h. Upon completion of the reaction (monitored by TLC with 15% MeOH/DCM eluent system), the solvent was removed on a rotatory evaporator. Next, the residue was dissolved in 3 mL ethanol and 3 drops of 12 M HCl were added. Subsequently, the reaction mixture was heated at 90° C. under reflux for 90 min. Afterwards, 6,9-dichloro-methoxyacridine (0.28 g, 1.0 mmol) was added to the mixture and further stirred at 90° C. under reflux and the course of reaction followed by TLC until little or no starting material was detected (around 12 h). On cooling to room temperature, the reaction mixture was diluted with cold water and neutralized to pH of 8-9 with 28% v/v ammonia solution. The alkaline solution was extracted with dichloromethane. The organic layer was washed with brine, concentrated in vacuum, and purified by column chromatography (gradient elution with 5% to 10% MeOH:DCM system) to afford A1 as orange reddish semisolid (0.08 g) in 78% yield; R_{f} 0.50 (2:8, MeOH:DCM); IR (cast film) vₘₐₓ=3272, 2923, 1629, 1254, 1231, 1032, 926, 816, 815, 775 cm⁻¹; ¹H NMR (500 MHz, CDCl₃) δ 7.99 (s, 1H), 7.91 (d, J=8.7 Hz, 1H), 7.80 (d, J=9.2 Hz, 1H), 7.29 (dd, J=9.4, 2.6 Hz, 1H), 7.11 (d, J=10.7 Hz, 1H), 7.03 (s, 1H), 6.77-6.70 (m, 2H), 6.49 (d, J=2.5 Hz, 1H), 3.63 (s, 3H), 3.53 (s, 2H), 2.75-2.32 (m, 8H), 2.27 (s, 3H), OH and NH protons were not observed; ¹³C NMR (125 MHz, CDCl₃) δ 155.9, 153.2, 147.8, 143.9, 137.0, 135.0, 125.0, 124.9, 122.0, 121.8, 120.0, 119.9, 119.7, 117.61, 116.8, 116.5, 116.1, 115.9, 100.3, 61.1, 55.2, 54.8 (2C), 52.4 (2C), 45.8; HRMS (ESI) calcd for C₂₆H₂₈ClN₄O₂[M+H]⁺ 463.1895; found 463.1890.

### 4-((6-Chloro-2-methoxyacridin-9-yl)amino)-2-((4-(3-methylbenzyl)piperazin-1-yl)methyl)phenol (A2)

The previous method was employed to synthesize A2 with the following stoichiometric amounts; 1-(3-methylbenzyl)piperazine (0.19 g, 1.0 mmol) and 6,9-dichloroacridine (0.28 g, 1.0 mmol) to afford it as orange reddish semisolid (0.14 g) in 73% yield; R_{f} 0.44 (1:9, MeOH:DCM); IR (cast film) vₘₐₓ=3258, 2921, 1629, 1560, 1493, 1253, 1133, 1007, 927, 826, 775 cm⁻¹; ¹H NMR (500 MHz, CDCl₃) δ 8.05 (s, 1H), 7.95 (d, J=9.2 Hz, 1H), 7.83 (d, J=9.2 Hz, 1H), 7.32 (d, J=7.6 Hz, 1H), 7.21 (t, J=7.5 Hz, 1H), 7.17-7.06 (m, 5H), 6.87 (d, J=7.1 Hz, 1H), 6.77 (d, J=8.6 Hz, 1H), 6.62 (d, J=1.9 Hz, 1H), 3.70 (s, 3H), 3.59 (s, 2H), 3.49 (s, 2H), 2.79-2.40 (m, 8H), 2.35 (s, 3H). OH and NH protons were not observed; ¹³C NMR (125 MHz, CDCl₃) δ 156.1, 153.9, 150.4, 146.8, 144.5, 137.9, 137.6, 136.3, 135.5, 129.9, 128.2, 127.9, 126.2, 125.4, 125.0, 122.1, 121.8, 120.6, 120.3, 119.2, 116.9, 116.5, 116.1, 115.9, 100.4, 62.8, 61.2, 55.4, 52.8 (2C), 52.5 (2C), 21.4; HRMS (ESI) calcd for C₃₃H₃₄ClN₄O₂[M+H]⁺ 553.2357; found 553.2365.

### 4-((6-Chloro-2-methoxyacridin-9-yl)amino)-2-((4-cyclohexylpiperazin-1-yl)methyl)phenol (A3)

The previous method was employed to synthesize A3 with the following stoichiometric amounts; 1-cyclohexyl piperazine (0.17 g, 1.0 mmol) and 6,9-dichloroacridine (0.28 g, 1.0 mmol) to afford it as orange reddish semisolid (0.13 g) in 81% yield; R_{f} 0.52 (0.5:9.5, MeOH:EtOAc); IR (cast film) vₘₐₓ=3313, 2918, 1736, 1560, 1468, 1255, 1235, 1181, 1032, 929, 829, 722 cm⁻¹; ¹H NMR (500 MHz, CDCl₃) δ 8.03 (d, J=1.4 Hz, 1H), 7.92 (d, J=9.6 Hz, 1H), 7.80 (d, J=9.5 Hz, 1H), 7.28 (d, J=5.7 Hz, 1H), 7.11 (d, J=9.1 Hz, 2H), 6.91 (d, J=9.3 Hz, 1H), 6.77 (d, J=8.5 Hz, 1H), 6.69 (s, 1H), 3.71 (s, 3H), 3.60 (s, 2H), 2.73-2.22 (m, 8H), 1.83 (m, 4H), 1.63 (d, J=12.8 Hz, 1H), 1.26-1.18 (m, 6H). OH and NH protons were not observed; ¹³C NMR (125 MHz, CDCl₃) δ 156.1, 153.8, 146.9, 144.5, 136.3, 135.5, 125.4-116.8 (10C), 101.3, 100.4, 98.8, 63.4, 61.3, 52.9 (2C), 48.7 (2C), 29.7, 28.9 (2C), 26.2, 25.8 (2C); HRMS (ESI) calcd for C₃₁H₃₅ClN₄O₂[M+H]⁺ 531.2521; found 531.2527.

### 4-((6-Chloro-2-methoxyacridin-9-yl)amino)-2-((4-(2-(dimethylamino)ethyl) piperazin-1-yl) methyl) phenol (A4)

The previous method was employed to synthesize A4 with the following stoichiometric amounts; 1-[2-(dimethylamino)ethyl] piperazine (0.16 g, 1.0 mmol) and 6,9-dichloroacridine (0.28 g, 1.0 mmol) to afford it as orange reddish semisolid (0.13 g) in 80% yield; R_{f} 0.40 (1.3:8.5:0.2, MeOH:DCM:Et₃N); IR (cast film) vₘₐₓ=3361, 2918, 1736, 1562, 1467, 1295, 1255, 1143, 1032, 945, 828, 763, 722 cm⁻¹; ¹H NMR (500 MHz, CDCl₃) δ 8.10 (s, 1H), 8.00 (s, 1H), 7.89 (d, J=9.1 Hz, 1H), 7.40 (d, J=9.8 Hz, 1H), 7.24 (d, J=8.0 Hz, 1H), 7.09 (s, 1H), 6.85 (dd, J=8.6, 2.7 Hz, 1H), 6.78 (d, J=8.6 Hz, 1H), 6.56 (d, J=2.3 Hz, 1H), 3.74 (s, 3H), 3.59 (s, 2H), 2.70-2.40 (m, 12H), 2.27 (s, 6H). OH and NH protons were not observed; ¹³C NMR (125 MHz, CDCl₃) δ 156.2, 153.6, 147.5, 144.0, 136.6, 135.2, 125.3, 125.1, 124.8, 122.0, 120.3, 120.2, 120.0, 119.8, 119.6, 117.5, 116.9, 116.3, 100.2, 61.2, 56.7, 56.7, 55.3, 53.3 (2C), 52.4 (2C), 45.8 (2C); HRMS (ESI) calcd for C₂₉H₃₅ClN₅O₂[M+H]⁺ 520.2474; found 520.2473.

### 4-((6-Chloro-2-methoxyacridin-9-yl)amino)-2-((4-(3,4-dichlorophenyl)piperazin-1-yl)methyl)phenol (A5)

The previous method was employed to synthesize A5 with the following stoichiometric amounts; 1-(3,4-dichlorophenyl) piperazine (0.23 g, 1.0 mmol), acetaminophen (0.15 g, 1.0 mmol), 37% formaldehyde (0.10 mL, 5 mmol) and 6,9-dichloroacridine (0.28 g, 1.0 mmol) to afford it as orange reddish semisolid (0.16 g) in 71% yield; R_{f} 0.56 (0.5:9.5, MeOH:DCM); IR (cast film) vₘₐₓ=3271, 2918, 1736, 1563, 1227, 1180, 1080, 1031, 803, 721 cm⁻¹; ¹H NMR (500 MHz, CDCl₃) δ 8.09 (s, 1H), 8.00 (s, 1H), 7.88 (d, J=9.2 Hz, 1H), 7.40 (d, J=9.3 Hz, 1H), 7.28 (d, J=8.7 Hz, 1H), 7.24 (d, J=9.0 Hz, 1H), 7.09 (s, 1H), 6.95 (d, J=2.8 Hz, 1H), 6.87 (dd, J=8.6, 2.7 Hz, 1H), 6.80 (d, J=8.6 Hz, 1H), 6.73 (dd, J=8.9, 2.8 Hz, 1H), 6.54 (d, J=2.6 Hz, 1H), 3.74 (s, 3H), 3.63 (s, 2H), 3.27-3.15 (m, 4H), 2.72-2.62 (m, 4H), OH and NH protons were not observed; ¹³C NMR (125 MHz, CDCl₃) δ 156.3, 153.1, 150.2, 147.9, 143.3, 137.0, 135.0, 132.9, 130.5, 125.2, 124.7, 122.9, 121.6, 121.4, 120.2, 120.0, 119.7, 117.9, 117.6, 117.0, 116.7, 116.2, 115.6, 115.5, 100.0, 61.2, 55.4 (2C), 52.2 (2C), 48.7; HRMS (ESI) calcd for C₃₁H₂₈Cl₃N₄O₂ [M+H]⁺ 593.1272; found 593.1272.

### 2-((4-(Bis(4-fluorophenyl)methyl)piperazin-1-yl)methyl)-4-((6-chloro-2-methoxyacridin-9-yl)amino)phenol (A6)

The previous method was employed to synthesize A6 with the following stoichiometric amounts; 1-bis (4-fluorophenyl)methylpiperazine (0.29 g, 1.0 mmol), acetaminophen (0.15 g, 1.0 mmol), 37% formaldehyde (0.10 mL, 5 mmol) and 6,9-dichloroacridine (0.28 g, 1.0 mmol) to afford it as orange reddish semisolid (0.19 g) in 65% yield; R_{f} 0.56 (0.5:9.5, MeOH:DCM); IR (cast film) vₘₐₓ=3275, 2919, 1737, 1566, 1255, 1217, 1181, 1032, 828, 722 cm⁻¹; ¹H NMR (500 MHz, CDCl₃) δ 8.07 (s, 1H), 7.98 (d, J=9.0 Hz, 1H), 7.84 (d, J=9.2 Hz, 1H), 7.34 (m, 6H), 7.18 (d, J=8.0 Hz, 1H), 7.06 (d, J=1.8 Hz, 1H), 6.97 (m, 5H), 6.83 (dd, J=8.5, 2.6 Hz, 1H), 6.75 (d, J=8.6 Hz, 1H), 6.53 (d, J=2.2 Hz, 1H), 4.23 (s, 1H), 3.69 (s, 3H), 3.57 (s, 2H), 2.74-2.21 (m, 8H); ¹³C NMR (125 MHz, CDCl₃) δ 161.9 (2C), 156.2, 153.5, 150.3, 147.7, 143.7, 137.8 (2C), 136.7, 135.0, 129.2 (4C), 125.2, 124.8, 121.9, 121.7, 120.2, 119.9, 119.7, 117.6, 116.8, 116.5, 116.1, 115.5 (4C), 100.0, 74.3, 61.1, 55.3, 52.6 (2C), 51.5 (2C); HRMS (ESI) calcd for C₃₈H₃₄ClF₂N₄O₂ [M+H]⁺ 651.2333; found 651.2328.

### 4-((6-Chloro-2-methoxyacridin-9-yl)amino)-2-((4-(4-hydroxyphenyl)piperazin-1-yl)methyl)phenol (A7)

The previous method was employed to synthesize A7 with the following stoichiometric amounts; 1-(4-hydroxyphenyl)piperazine (0.30 g, 1.0 mmol), acetaminophen (0.15 g, 1.0 mmol), 37% formaldehyde (0.10 mL, 5 mmol) and 6,9-dichloroacridine (0.28 g, 1.0 mmol) to afford it as orange reddish semisolid (0.17 g) in 59% yield; R_{f} 0.58 (1.5:8.5, MeOH:DCM); IR (cast film) vₘₐₓ=3334, 2918, 1737, 1564, 1468, 1235, 1181, 1031, 930, 830, 722 cm⁻¹; ¹H NMR (500 MHz, CD₃OD) δ 8.07 (d, J=9.3 Hz, 1H), 7.87 (d, J=1.8 Hz, 1H), 7.82 (d, J=9.3 Hz, 1H), 7.51-7.46 (m, 1H), 7.41 (d, J=2.5 Hz, 1H), 7.29-7.24 (m, 1H), 7.04 (dd, J=8.7, 2.4 Hz, 1H), 6.93-6.89 (m, 1H), 6.88-6.81 (m, 3H), 6.70 (dd, J=8.9, 2.3 Hz, 2H), 3.72 (s, 3H), 3.61 (s, 2H), 3.11-2.99 (m, 4H), 2.82-2.67 (m, 4H), OH and NH protons were not observed; ¹³C NMR (125 MHz, CDCl₃) δ 156.3, 153.5, 152.1, 150.2, 150.1, 145.3, 145.2, 144.0, 125.3, 124.7, 122.5, 121.9, 121.5, 120.4, 120.0, 118.8 (2C), 118 (2C), 118.4, 118.1, 117.0, 116.5, 116.1, 115.9, 61.3, 55.4, 52.7 (2C), 50.7 (2C); HRMS (ESI) calcd for C₃₁H₃₀ClN₄O₃[M+H]⁺ 541.2001; found 541.2010.

### 4-((6-Chloro-2-methoxyacridin-9-yl)amino)phenol (A8)

Synthesis of A8 was carried out by mixing 4-aminophenol (0.11 g, 1.0 mmol) and 6,9-dichloroacridine (0.28 g, 1.0 mmol) in 10 mL single neck round bottom flask and stirred at 90° C. under reflux. The course of reaction followed by TLC until little or no starting material was detected (around 12 h). On cooling to room temperature, the reaction mixture was diluted with cold water and neutralized to pH of 8-9 with 28% v/v ammonia solution. The alkaline solution was extracted with dichloromethane. The organic layer was washed with brine, concentrated in vacuum, and purified by column chromatography (gradient elution with 1% to 5% MeOH:DCM system) to afford it as orange reddish semisolid in 73% yield; R_{f} 0.47 (1.5:8.5, MeOH:DCM); IR (cast film) vₘₐₓ=3325, 2918, 1737, 1562, 1468, 1236, 1180, 1103, 1050, 722 cm⁻¹; ¹H NMR (500 MHz, CD₃OD) δ 8.12 (d, J=9.4 Hz, 1H), 7.84-7.83 (m, 1H), 7.78 (d, J=9.3 Hz, 1H), 7.61 (dd, J=9.3, 2.6 Hz, 1H), 7.48 (d, J=1.5 Hz, 1H), 7.35 (dd, J=9.4, 2.1 Hz, 1H), 7.27-7.22 (m, 2H), 6.95-6.91 (m, 2H), 3.88 (s, 1H), 3.69 (s, 3H). OH proton was not observed; ¹³C NMR (125 MHz, CD₃OD) δ 158.9, 157.7, 141.7, 137.9, 133.2, 129.8, 128.4, 127.8 (2C), 126.6, 125.7, 122.2, 120.1, 119.3, 117.8 (2C), 116.1, 113.3, 104.5, 56.1; HRMS (ESI) calcd for C₂₀H₁₆ClN₂O₂[M+H]⁺ 351.0908; found 351.0896.

7. Cytotoxicity Profile of A4

See FIG. 8, which depicts cell proliferation of HCT116 cells treated with compounds A4 (active inhibitor) and A5 (non-active inhibitor) using MTS assay. Also see FIG. 9.

### REFERENCES

1. Jordheim L P, Barakat K H, Heinrich-Balard L, Matera E-L, Cros-Perrial E, Bouledrak K, et al. Small molecule inhibitors of ERCC1-XPF protein-protein interaction synergize alkylating agents in cancer cells. Mol Pharmacol [Internet]. 2013 [cited 2015 Sep. 6]; 84:12-24. Available from: http://molpharm.aspetjournals.org/content/84/1/12.short
2. Anandakrishnan R, Aguilar B, OnufrievA V. H++3.0: automating pK prediction and the preparation of biomolecular structures for atomistic molecular modeling and simulations. Nucleic Acids Res [Internet]. 2012 [cited 2015 Mar. 25]; 40:W537-41. Available from: http://www.pubmedcentral.nih.gov/articlerender.fcgi?artid=3394296&tool=pmcentrez&rendertype=abstract
3. Case D A, Babin V, Berryman J T, Betz R M, Cai Q, Cerutti D S, et al. Amber 14 [Internet]. [cited 2015 Oct. 31]. Available from: http://ambermd.org/
4. Maier J A, Martinez C, Kasavajhala K, Wickstrom L, Hauser K E, Simmerling C. ff14SB: Improving the Accuracy of Protein Side Chain and Backbone Parameters from ff99SB. J Chem Theory Comput [Internet]. 2015 [cited 2016 May 27]; 11:3696-713. Available from: http://pubs.acs.org/doi/abs/10.1021/acs.jctc.5b00255
5. Li P, Roberts B P, Chakravorty D K, Merz K M. Rational Design of Particle Mesh Ewald Compatible Lennard-Jones Parameters for +2 Metal Cations in Explicit Solvent. J Chem Theory Comput [Internet]. 2013 [cited 2016 May 27]; 9:2733-48. Available from: http://pubs.acs.org/doi/abs/10.1021/ct400146w
6. Li P, Song L F, Merz K M. Systematic parameterization of monovalent ions employing the nonbonded model. J Chem Theory Comput [Internet]. 2015 [cited 2016 May 27]; 11:1645-57. Available from: http://pubs.acs.org/doi/abs/10.1021/ct500918t
7. Salomon-Ferrer R, Gotz A W, Poole D, Le Grand S, Walker R C. Routine Microsecond Molecular Dynamics Simulations with AMBER on GPUs. 2. Explicit Solvent Particle Mesh Ewald. J Chem Theory Comput [Internet]. American Chemical Society; 2013 [cited 2014 Oct. 19]; 9:3878-88. Available from: http://dx.doi.org/10.1021/ct400314y
8. Bolton E E, Wang Y, Thiessen P A, Bryant S H. Chapter 12 PubChem: Integrated Platform of Small Molecules and Biological Activities. Annu Rep Comput Chem [Internet]. 2008; 4:217-41. Available from: http://www.sciencedirect.com/science/article/pii/S1574140008000121
9. Schrödinger. LigPrep [Internet]. Schrödinger, LLC, New York, N.Y. 2013 [cited 2014 Jan. 24]. Available from: http://www.schrodinger.com/productpage/14/10/
10. Humphrey W, Dalke A, Schulten K. VMD: Visual molecular dynamics. J Mol Graph [Internet]. 1996 [cited 2014 Jul. 10]; 14:33-8. Available from: http://www.sciencedirect.com/science/article/pii/0263785596000185
11. Morris G M, Huey R, Lindstrom W, Sanner M F, Belew R K, Goodsell D S, et al. AutoDock4 and AutoDockTools4: Automated docking with selective receptor flexibility. J Comput Chem [Internet]. 2009 [cited 2013 Dec. 24]; 30:2785-91. Available from: http://www.pubmedcentral.nih.gov/articlerender.fcgi?artid=2760638&tool=pmcentrez&rendertype=abstract
12. Morris G, Goodsell D. Automated docking using a Lamarckian genetic algorithm and an empirical binding free energy function. J Comput Chem [Internet]. 1998 [cited 2013 Oct. 23]; 1639-62. Available from: http://ub.cbm.uam.es/publications/teaching/curso_verano_uah_2011/autodock4.pdf
13. Huey R, Morris G M, Olson A J, Goodsell D S. A semiempirical free energy force field with charge-based desolvation. J Comput Chem [Internet]. 2007 [cited 2013 Dec. 25]; 28:1145-52. Available from: http://www.ncbi.nlm.nih.gov/pubmed/17274016
14. Wang J, Wang W, Kollmann P, Case D. Antechamber, An Accessory Software Package For Molecular Mechanical Calculation. J Comput Chem. 2005; 25:1157-74.
15. Wang J, Wolf R M, Caldwell J W, Kollman P A, Case D A. Development and testing of a general amber force field. J Comput Chem [Internet]. 2004 [cited 2014 Jan. 26]; 25:1157-74. Available from: http://www.ncbi.nlm.nih.gov/pubmed/15116359
16. Gentile F, Tuszynski J A, Barakat K H. New Design of Nucleotide Excision Repair (NER) Inhibitors for Combination Cancer Therapy. J Mol Graph Model [Internet]. 2016 [cited 2016 Feb. 29]; 65:71-82. Available from: http://www.sciencedirect.com/science/article/pii/S1093326316300213
17. Miller B R, McGee T D, Swails J M, Homeyer N, Gohlke H, Roitberg A E. MMPBSA.py: An Efficient Program for End-State Free Energy Calculations. J Chem Theory Comput [Internet]. 2012 [cited 2014 May 26]; 8:3314-21. Available from: http://pubs.acs.org/doi/abs/10.1021/ct300418h
18. Genheden S, Ryde U. The MM/PBSA and MM/GBSA methods to estimate ligand-binding affinities. Expert Opin Drug Discov [Internet]. Taylor & Francis; 2015; 10:449-61. Available from: http://dx.doi.org/10.1517/17460441.2015.1032936
19. Onufriev A, Bashford D, Case D A. Exploring protein native states and large-scale conformational changes with a modified generalized born model. Proteins [Internet]. 2004 [cited 2014 Feb. 26]; 55:383-94. Available from: http://www.ncbi.nlm.nih.gov/pubmed/15048829
20. Weiser J, Shenkin P S, Still W C. Approximate atomic surfaces from linear combinations of pairwise overlaps (LCPO). J Comput Chem [Internet]. 1999 [cited 2015 Jul. 22]; 20:217-30. Available from: http://doi.wiley.com/10.1002/%28SICI%291096-987X%2819990130%2920%3A2%3C217%3A %3AAID-JCC4%3E3.0.CO%3B2-A

### Example 3—First, Second, Third and Fourth Generation of F06 Analogues

1. Computational Methods

The computational workflow used for virtual screening of all the compounds was the same adopted for the first generation (see paragraph [00147]-[00150]). Computational results are reported below, where available.

2. Molecular Structures

2.1 First Generation (Gen A)

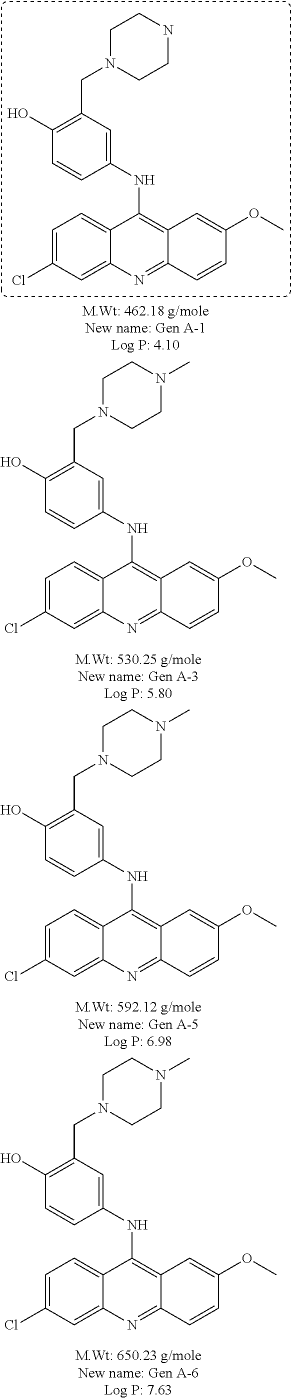 [SMILES: COc1ccc2nc3cc(Cl)ccc3c(Nc3ccc(O)c(CN4CCNCC4)c3)c2c1 ; COc1ccc2nc3cc(Cl)ccc3c(Nc3ccc(O)c(CN4CCN(C)CC4)c3)c2c1 ; COc1ccc2nc3cc(Cl)ccc3c(Nc3ccc(O)c(CN4CCN(C)CC4)c3)c2c1 ; COc1ccc2nc3cc(Cl)ccc3c(Nc3ccc(O)c(CN4CCN(C)CC4)c3)c2c1]

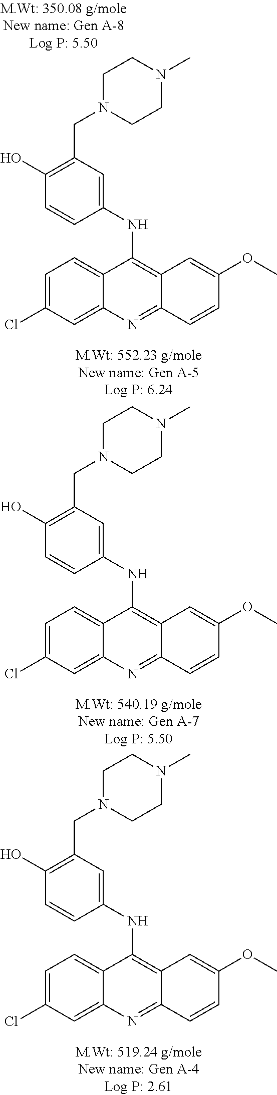 [SMILES: COc1ccc2nc3cc(Cl)ccc3c(Nc3ccc(O)c(CN4CCN(C)CC4)c3)c2c1 ; COc1ccc2nc3cc(Cl)ccc3c(Nc3ccc(O)c(CN4CCN(C)CC4)c3)c2c1 ; COc1ccc2nc3cc(Cl)ccc3c(Nc3ccc(O)c(CN4CCN(C)CC4)c3)c2c1]

2.2 Second Generation (Gen B)

2.2.1 Chemical Structures

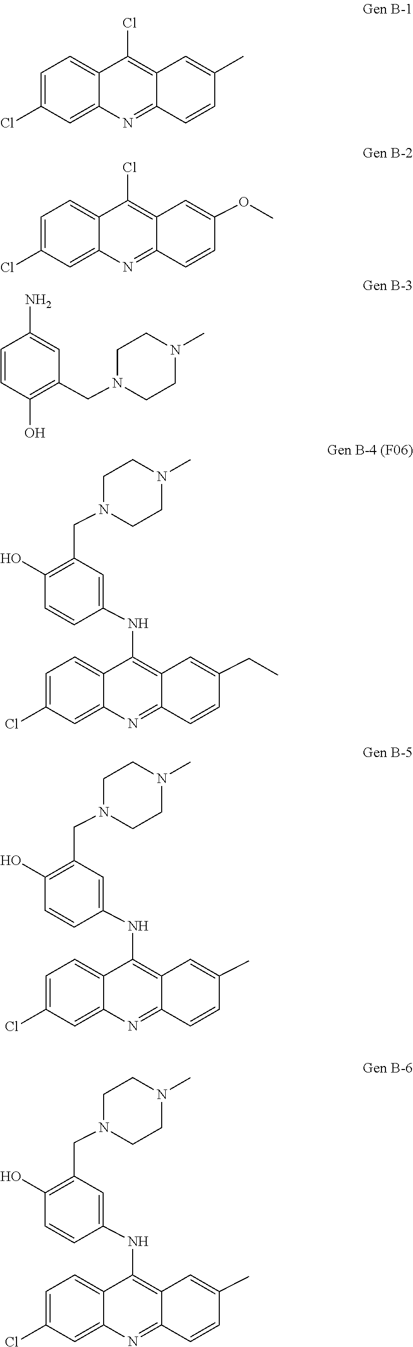

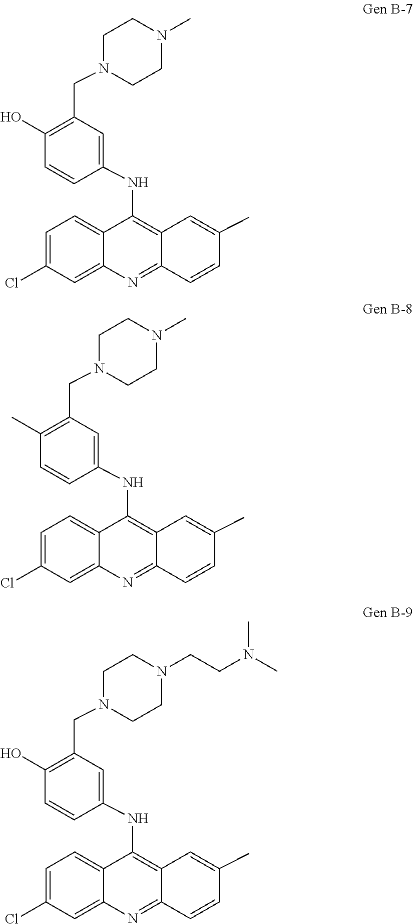

2.1.2 Computational Results

TABLE 1
Computational results for second generation compounds (ha = heavy atom).

| | | Ligand | | |
|---|---|---|---|---|
| | MOE Dock score | efficiency | MM-GBSA | |
| Molecule | (kcal/mol) | (kcal/mol/ha) | (kcal/mol) | LogP |
| B-4 | −6.70 | −0.20 | −17.78 | 4.10 |
| B-6 | −8.56 | −0.21 | −14.19 | 5.31 |
| B-1 | −5.21 | −0.31 | −7.11 | 4.40 |
| B-5 | −5.90 | −0.18 | −3.79 | 3.79 |
| B-7 | −5.17 | −0.13 | −8.15 | 5.93 |
| B-9 | −7.00 | −0.19 | −12.44 | 2.31 |

1. Results for the 3^{rd} Generation Compounds Gen C

1.1 Summary of Compounds Gen C

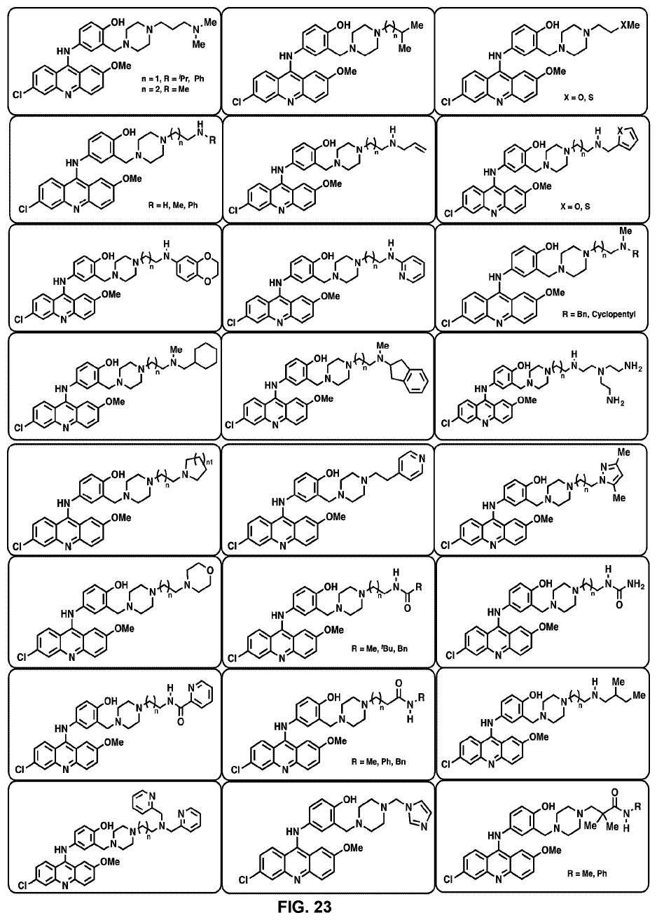

In comparison to Gen A-1 (F06), compound Gen A-4 showed significantly better inhibitory properties against ERCC1-XPF. It is not clarified yet, if the improvement in activity was positively affected by the additional methyl group, the dimethylamino group, the amine functionality itself, an additional heteroatom or the introduced linker. Because of that, a set of 3^{rd} generation compounds Gen C was developed, including branched as well as linear aliphatic, aromatic and heteroaromatic substituents at the amine functionality, nitrogen heterocycles, amides, different heteroatoms connected to the linker and an extended linker size between the aminophenol moiety and the piperazine (FIG. 23).

1.2 In Silico Studies

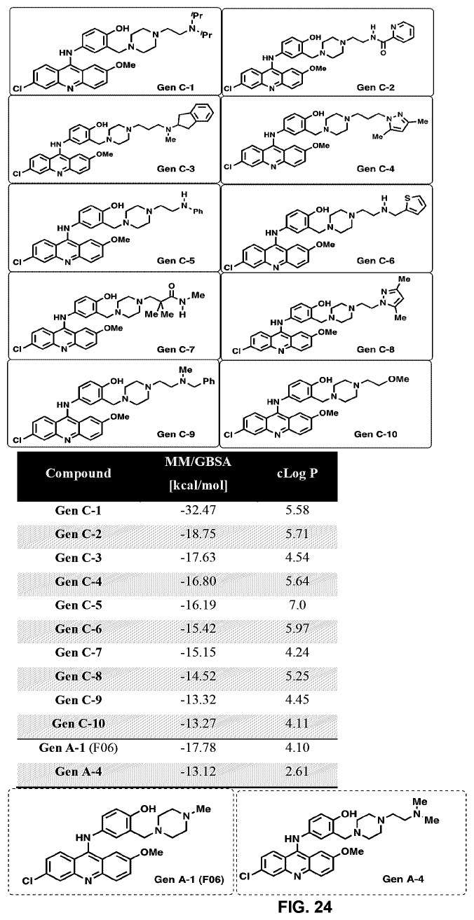

The in silico screening predicted the calculation of average binding energies and log P values for all compounds, shown in FIG. 23. This virtual screening gave a list of top ten compounds, which all showed promising binding energies. An outstanding value was calculated for Gen C-1 (FIG. 24).

2) TFA-Salts of Synthesized Compounds for 3^{rd} Generation Gen C (and Gen A)

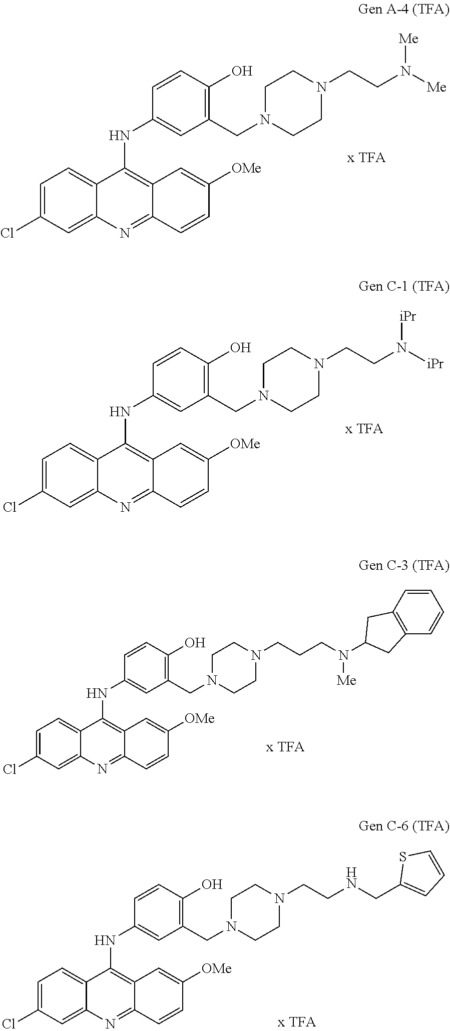 [SMILES: COc1ccc2nc3cc(Cl)ccc3c(Nc3ccc(O)c(CN4CCN(CCN(C)C)CC4)c3)c2c1 ; COc1ccc2nc3cc(Cl)ccc3c(Nc3ccc(O)c(CN4CCN(CCN(C(C)C)C(C)C)CC4)c3)c2c1 ; COc1ccc2nc3cc(Cl)ccc3c(Nc3ccc(O)c(CN4CCN(CCCN(C)C5Cc6ccccc6C5)CC4)c3)c2c1 ; COc1ccc2nc3cc(Cl)ccc3c(Nc3ccc(O)c(CN4CCN(CCNCc5cccs5)CC4)c3)c2c1]

3) General Strategy for the Synthesis Of 3^{rd} Generation Compounds Gen C

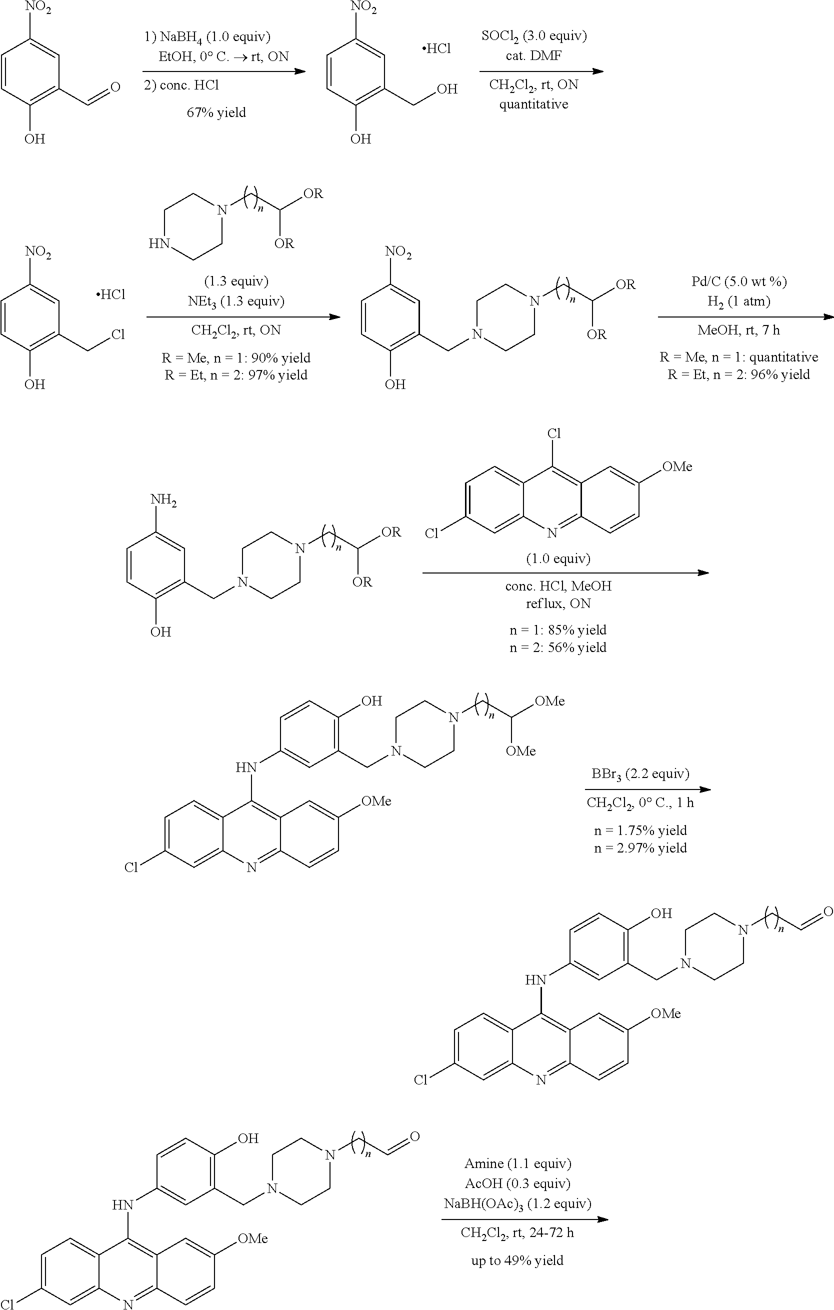

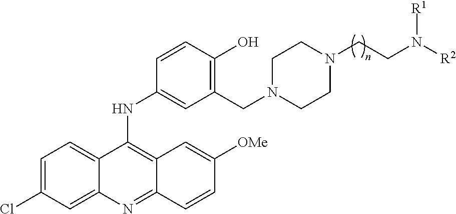

4) Alternative Synthesis for Gen C-1

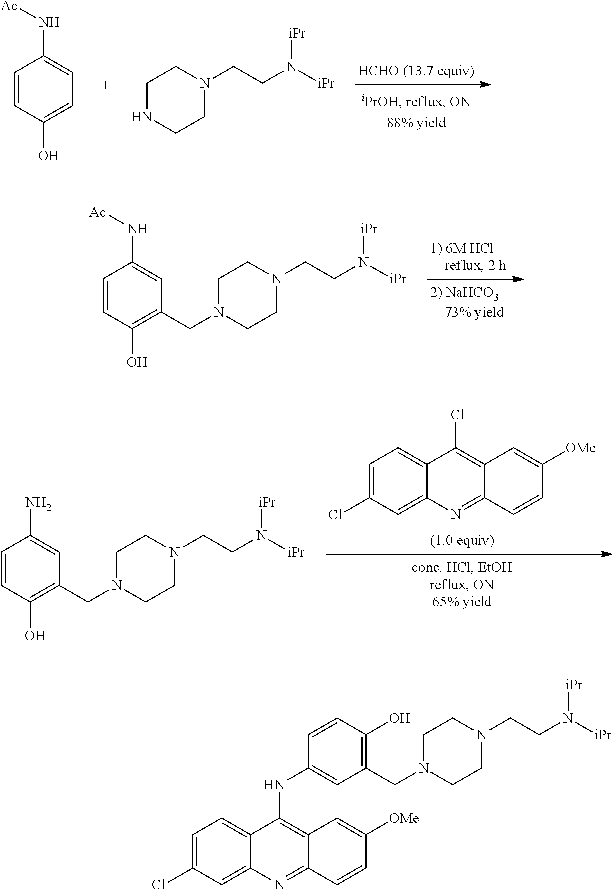 [SMILES: CC(=O)Nc1ccc(O)cc1 ; CC(C)N(CCN1CCNCC1)C(C)C ; CC(=O)Nc1ccc(O)c(CN2CCN(CCN(C(C)C)C(C)C)CC2)c1 ; CC(C)N(CCN1CCN(Cc2cc(N)ccc2O)CC1)C(C)C ; COc1ccc2nc3cc(Cl)ccc3c(Cl)c2c1 ; COc1ccc2nc3cc(Cl)ccc3c(Nc3ccc(O)c(CN4CCN(CCN(C(C)C)C(C)C)CC4)c3)c2c1]

5) Synthesis for Gen C-7

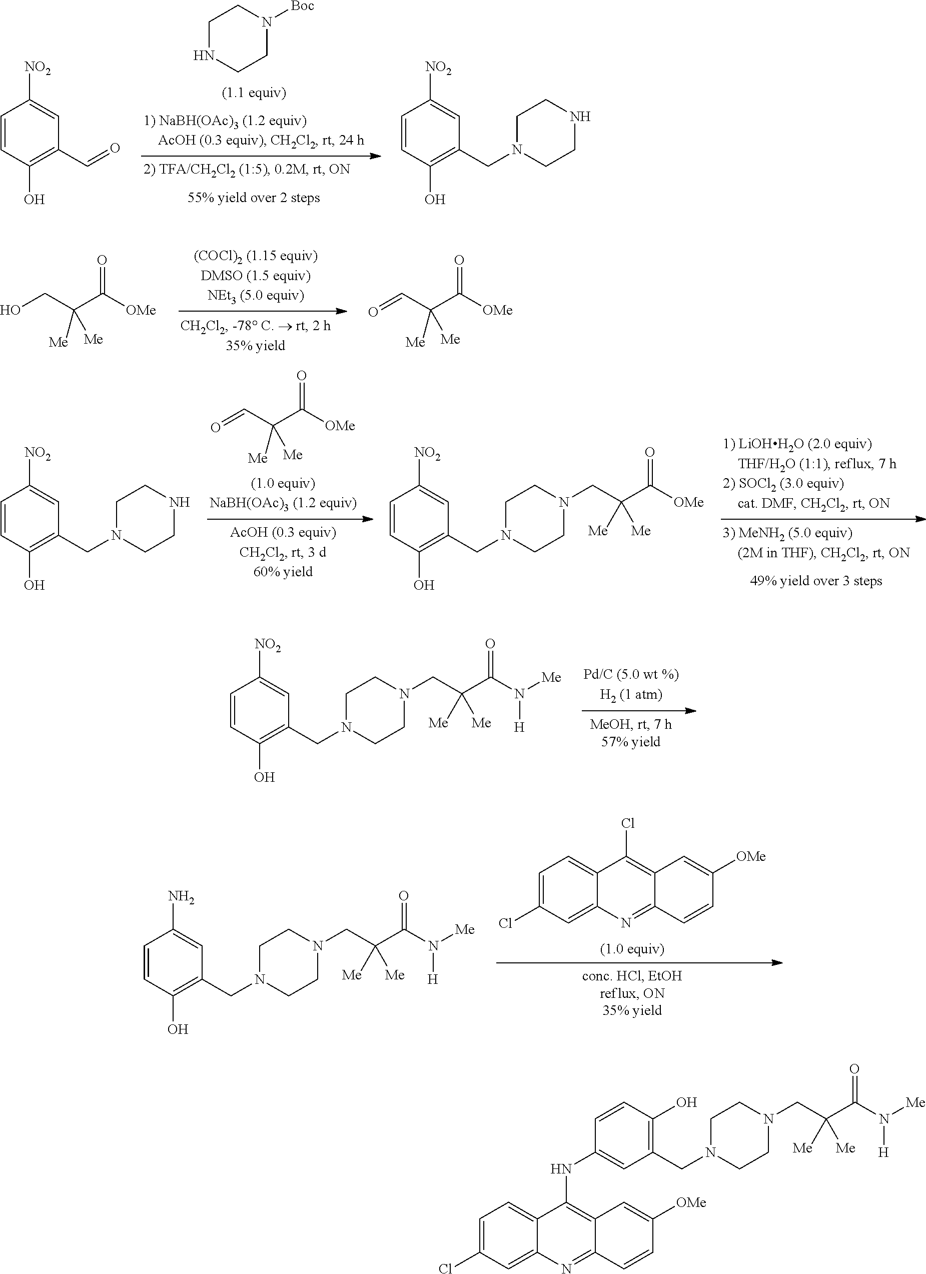

6) Synthesis for Gen C-10

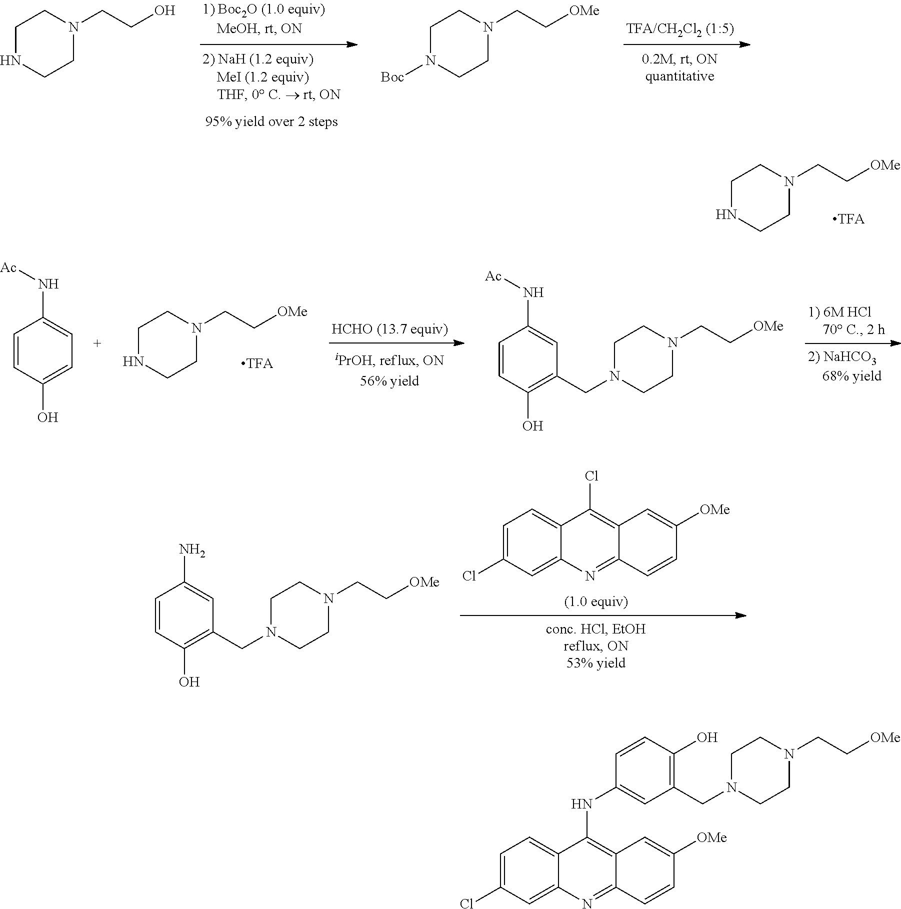

Synthesis of 3^{rd} Generation Compounds Gen C with Late Stage Reductive Amination

### Synthesis of 2-(Hydroxymethyl)-4-nitrophenol hydrochloride 1

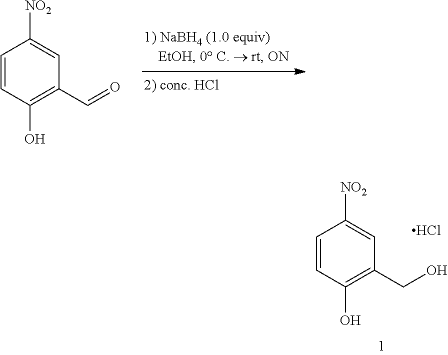 [SMILES: O=Cc1cc([N+](=O)[O-])ccc1O ; O=[N+]([O-])c1ccc(O)c(CO)c1 ; Cl]

According to the procedure of: C. Arenz, A. Giannis, Eur. J. Org. Chem. 2001, 137-140. In an oven-dried flask under nitrogen atmosphere, 2-hydroxy-5-nitrobenzaldehyde (2.55 g, 15.2 mmol, 1.0 equiv) was dissolved in 30 mL EtOH and cooled to 0° C. Sodium boronhydride (577 mg, 15.2 mmol, 1.0 equiv) was added in 3 portions. After stirring at 0° C. for 30 min, the reaction mixture was warmed to room temperature (rt) and stirred ON. HCl (12 M) was added to the yellow solution until the mixture appeared colourless. The formed precipitate was separated by filtration. Recrystallization in 20 mL H₂O isolated compound 1 in 2.08 g (10.1 mmol, 67%) as a colourless solid.

R_{F} (CH₂Cl₂/MeOH 5:1)=0.49. IR (film): v (cm⁻¹)=3524; 3460; 3086; 2963; 2904; 2822; 2762; 2688; 2623; 2571; 1917; 1617; 1599; 1519; 1488; 1465; 1438; 1399; 1363; 1307; 1198; 1178; 1140; 1092; 987; 957; 935; 912; 837; 752; 735. ¹H NMR (500 MHz, CD₃OD): δ (ppm)=8.25 (d, J=2.8 Hz, 1H), 8.02 (dd, J=8.9 Hz, J=2.9 Hz, 1H), 6.85 (d, J=8.9 Hz, 1H), 4.64 (s, 2H). HR-MS (ESI): calc. for C₇H₆NO₄ ([M−H]⁻): 168.0302; found: 168.0302. C₇H₇NO₄ (169.04). C₇H₈ClNO₄ (205.01).

### Synthesis of 2-(Chloromethyl)-4-nitrophenol hydrochloride 2

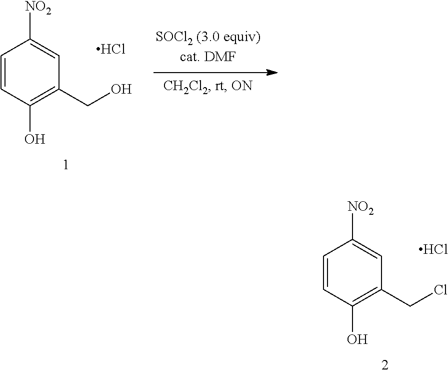 [SMILES: O=[N+]([O-])c1ccc(O)c(CO)c1 ; Cl ; O=[N+]([O-])c1ccc(O)c(CCl)c1 ; Cl]

In an oven-dried flask under nitrogen atmosphere, 2-(Hydroxymethyl)-4-nitrophenol hydrochloride 1 (460 mg, 2.24 mmol, 1.0 equiv) was dissolved in 10 mL CH₂Cl₂. Three drops of DMF were added, followed by the dropwise addition of thionyl chloride (488 μL, 800 mg, 6.72 mmol, 3.0 equiv). The reaction mixture was stirred at rt ON and the solvent was removed in vacuo. 500 mg (2.24 mmol, quant.) of compound 2 were isolated as a beige solid.

R_{F} (CH₂Cl₂/MeOH 5:1)=0.78. IR (film): v (cm⁻¹)=3218; 3091; 2967; 2933; 1655; 1618; 1595; 1524; 1498; 1435; 1387; 1342; 1292; 1235; 1157; 1130; 1087; 941; 917; 834; 752; 673. ¹H NMR (500 MHz, CDCl₃): δ (ppm)=8.28 (d, J=2.7 Hz, 1H), 8.17-8.07 (m, 1H), 7.07-6.94 (m, 1H), 4.69 (s, 1H). HR-MS (ESI): calc. for C₇H₅ClNO₃ ([M−H]⁻): 185.9963; found: 185.9969. C₇H₆ClNO₃ (187.00). C₇H₇Cl₂NO₃ (222.98).

General Procedure 1: Nucleophilic Substitution for the Synthesis of Compounds 4

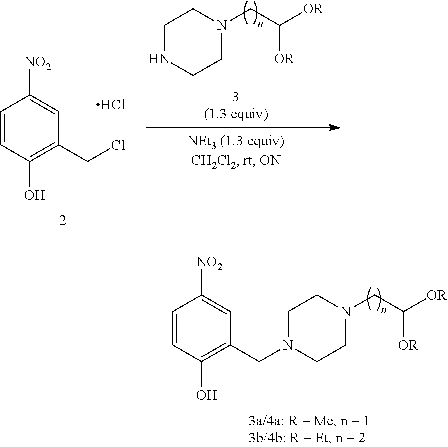

In an oven-dried flask under nitrogen atmosphere, 2-(chloromethyl)-4-nitrophenol hydrochloride 2 (1.0 equiv) was dissolved in CH₂Cl₂ (0.2 M). Triethylamine (1.3 equiv) was added dropwise at rt, followed by the addition of compound 3 (1.3 equiv). The reaction mixture was stirred at rt ON and the solvent was removed in vacuo. The crude mixture was purified by column chromatography using CH₂Cl₂/2% NEt₃ with MeOH as gradient elution (1%→5%).

### 2-((4-(2,2-Dimethoxyethyl)piperazin-1-yl)methyl)-4-nitrophenol 4a

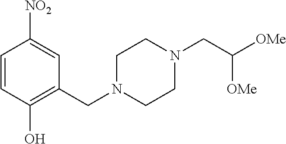 [SMILES: COC(CN1CCN(Cc2cc([N+](=O)[O-])ccc2O)CC1)OC]

The reaction was performed with 353 mg (1.58 mmol, 1.0 equiv) 2-(chloromethyl)-4-nitrophenol hydrochloride 2, 285 μL (209 mg, 2.06 mmol) triethylamine and 359 mg (2.06 mmol) 1-(2,2-dimethoxyethyl)piperazine 3a in 8.0 mL CH₂Cl₂ (in accordance with general procedure 1). Appearance: Yellow solid. Yield: 462 mg, 1.42 mmol, 90%. R_{F} (CH₂Cl₂/MeOH 5:1)=0.97. IR (film): v (cm⁻¹)=2946; 2830; 1675; 1619; 1590; 1523; 1480; 1458; 1406; 1337; 1284; 1157; 1133; 1082; 1008; 968; 930; 823; 792; 771; 753; 720; 661. ¹H-NMR (400 MHz, CDCl₃): δ (ppm)=8.09 (dd, J=9.0 Hz, J=2.8 Hz, 1H), 7.95 (d, J=2.8 Hz, 1H), 6.85 (d, J=9.0 Hz, 1H), 4.51 (t, J=5.2 Hz, 1H), 3.79 (s, 2H), 3.36 (s, 6H), 2.87-2.36 (m, 10H). HR-MS (ESI): calc. for C₁₅H₂₄N₃O₅ ([M+H]⁺): 326.1710; found: 326.1707. C₁₅H₂₃N₃O₅ (325.16).

### 2-((4-(3,3-Diethoxypropyl)piperazin-1-yl)methyl)-4-nitrophenol 4b

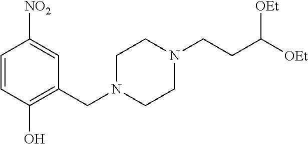 [SMILES: CCOC(CCN1CCN(Cc2cc([N+](=O)[O-])ccc2O)CC1)OCC]

The reaction was performed with 652 mg (2.93 mmol) 2-(chloromethyl)-4-nitrophenol hydrochloride 2, 527 μL (385 mg, 3.80 mmol) triethylamine and 822 mg (3.80 mmol) 1-(3,3-diethoxypropyl)piperazine 3b in 14 mL CH₂Cl₂ (in accordance with general procedure 1). Appearance: Yellow solid. Yield: 1.04 g, 2.83 mmol, 97%. R_{F} (CH₂Cl₂/MeOH 5:1)=0.98. IR (film): v (cm⁻¹)=2973; 2881; 2824; 1619; 1590; 1523; 1480; 1452; 1374; 1337; 1283; 1156; 1134; 1088; 1062; 1010; 997; 930; 903; 823; 771; 753; 719; 661. ¹H-NMR (400 MHz, CDCl₃): δ (ppm)=8.11 (dd, J=9.0 Hz, J=2.8 Hz, 1H), 7.96 (dt, J=2.8 Hz, J=0.9 Hz 1H), 6.86 (d, J=9.0 Hz, 1H), 4.58 (t, J=5.6 Hz, 1H), 3.80 (s, 2H), 3.64 (dd, J=9.4 Hz, J=7.1 Hz, 2H), 3.51 (dd, J=9.4 Hz, J=7.0 Hz, 2H), 3.22-2.29 (m, 10H), 1.82 (q, J=6.8 Hz, 2H), 1.20 (t, J=7.0 Hz, 6H). HR-MS (ESI): calc. for C₁₈H₃₀N₃O₅ ([M+H]⁺): 368.2180; found: 368.2180. C₁₈H₂₉N₃O₅ (367.21).

General Procedure 2: Hydrogenation for the Synthesis of Compounds 5

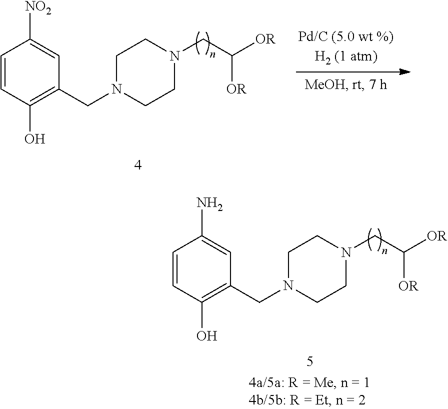

In an oven-dried flask under nitrogen atmosphere, Pd/C (5.0 wt %) was suspended in MeOH (0.05 M) and A4 (1.0 equiv) was added. The resulting dark suspension was stirred under 1 atm for 7 h. Subsequently, the crude mixture was filtered over Celite® and washed with MeOH (1.0 mL/0.1 mmol). The solvent was removed in vacuo and the residue was purified by column chromatography using CH₂Cl₂/2% NEt₃ with MeOH as gradient elution (2%→10%).

### 4-Amino-2-((4-(2,2-dimethoxyethyl)piperazin-1-yl)methyl)phenol 5a

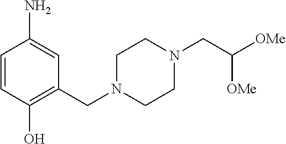 [SMILES: COC(CN1CCN(Cc2cc(N)ccc2O)CC1)OC]

The reaction was performed with 179 mg (0.550 mmol) 2-((4-(2,2-dimethoxyethyl)piperazin-1-yl)methyl)-4-nitrophenol 4a and 15 mg Pd/C in 11 mL MeOH (in accordance with general procedure 2). Appearance: Light yellow oil. Yield: 162 mg, 0.550 mmol, quant. R_{F} (CH₂Cl₂/MeOH 5:1)=0.72. IR (film): v (cm⁻¹)=3422; 3350; 3224; 2944; 2827; 2710; 1631; 1498; 1457; 1406; 1368; 1349; 1336; 1297; 1279; 1245; 1220; 1154; 1134; 1069; 1009; 993; 967; 934; 865; 820; 773; 735; 706. ¹H-NMR (400 MHz, CDCl₃): δ (ppm)=6.65 (d, J=8.4 Hz, 1H), 6.55 (dd, J=8.4 Hz, J=2.8 Hz, 1H), 6.38 (d, J=2.8 Hz, 1H), 4.50 (t, J=5.2 Hz, 1H), 3.60 (s, 2H), 3.35 (d, J=0.6 Hz, 6H), 2.74-2.44 (m, 8H), 1.08 (t, J=7.2 Hz, 2H). HR-MS (ESI): calc. for C₁₅H₂₆N₃O₃ ([M+H]⁺): 296.1969; found: 296.1970. C₁₅H₂₅N₃O₃ (295.19).

### 4-Amino-2-((4-(3,3-diethoxypropyl)piperazin-1-yl)methyl) Phenol 5b

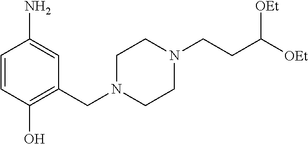 [SMILES: CCOC(CCN1CCN(Cc2cc(N)ccc2O)CC1)OCC]

The reaction was performed with 438 mg (1.19 mmol) 2-((4-(3,3-diethoxypropyl)piperazin-1-yl)methyl)-4-nitrophenol 4b and 35 mg Pd/C in 22 mL MeOH (in accordance with general procedure 2). Appearance: Light yellow oil. Yield: 385 mg, 1.14 mmol, 96%. R_{F} (CH₂Cl₂/MeOH 5:1)=0.78. IR (film): v (cm⁻¹)=3421; 3353; 3215; 2973; 2942; 2878; 2820; 1632; 1498; 1458; 1346; 1313; 1245; 1136; 1061; 996; 931; 868; 816; 774. ¹H-NMR (400 MHz, CDCl₃): δ (ppm)=6.65 (d, J=8.4 Hz, 1H), 6.55 (dd, J=8.4 Hz, J=2.8 Hz, 1H), 6.38 (d, J=2.8 Hz, 1H), 4.56 (t, J=5.6 Hz, 1H), 3.73-3.56 (m, 4H), 3.49 (dq, J=9.4 Hz, J=7.0 Hz, 2H), 2.72-2.32 (m, 10H), 1.81 (d, J=6.6 Hz, 2H), 1.19 (t, J=7.0 Hz, 6H). HR-MS (ESI): calc. for C₁₈H₃₂N₃O₃ ([M+H]⁺): 338.2440; found: 338.2430. C₁₈H₃₁N₃O₃ (337.24).

General Procedure 3: Nucleophilic Aromatic Substitution for the Synthesis of Compounds 6

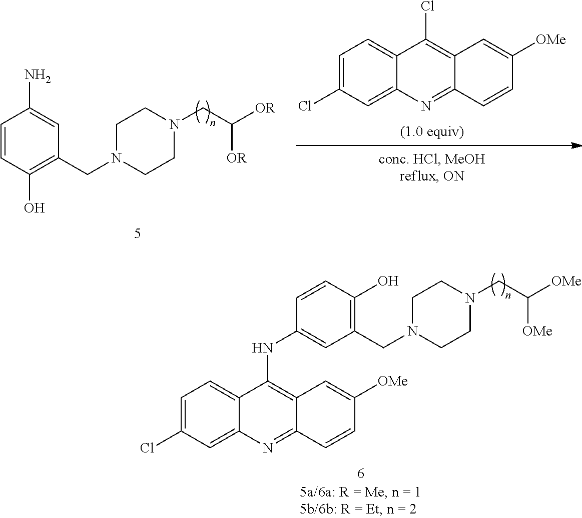

6,9-Dichloro-2-methoxyacridine (1.0 equiv) and compound 5 (1.0 equiv) were dissolved in MeOH (0.05 M). Subsequently, conc. HCl (1 drop/0.5 mmol) was added to the yellow suspension, which resulted in the formation of an orange solution. The reaction mixture was heated to 80° C. for 24 h. After cooling down, a saturated NaHCO₃ solution was added (until pH=8-9), and the mixture was extracted with CH₂Cl₂ (3×50 mL/1.0 mmol). The combined organic phases were dried over magnesium sulfate. The solution was filtered and the solvent was removed in vacuo. The residue was purified by column chromatography using CH₂Cl₂/2% NEt₃ with MeOH as gradient elution (1%→2%).

### 4-((6-Chloro-2-methoxyacridin-9-yl)amino)-2-((4-(2,2-dimethoxyethyl) piperazin-1-yl)methyl)phenol 6a/Gen C-11

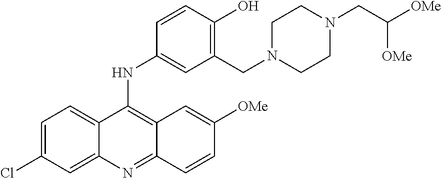 [SMILES: COc1ccc2nc3cc(Cl)ccc3c(Nc3ccc(O)c(CN4CCN(CC(OC)OC)CC4)c3)c2c1]

The reaction was performed with 540 mg (1.94 mmol) 6,9-dichloro-2-methoxyacridine and 573 mg (1.94 mmol) 4-amino-2-((4-(2,2-dimethoxyethyl)-piperazin-1-yl)methyl)phenol 5a in 40 mL MeOH (in accordance with general procedure 3). Appearance: Orange solid. Yield: 888 mg, 1.66 mmol, 85%. R_{F} (CH₂Cl₂/MeOH 5:1)=0.69. IR (film): v (cm⁻¹)=3246; 2926; 2850; 2830; 1736; 1631; 1606; 1561; 1519; 1494; 1467; 1438; 1420; 1364; 1334; 1303; 1254; 1234; 1134; 1072; 1032; 1010; 994; 967; 929; 873; 828; 803; 774; 735; 703. ¹H-NMR (400 MHz, CDCl₃): δ (ppm)=8.12 (s, 1H), 8.03 (s, 1H), 7.88 (d, J=9.3 Hz, 1H), 7.39 (d, J=9.3 Hz, 1H), 7.06 (s, 1H), 6.83 (dd, J=8.6 Hz, J=2.7 Hz, 1H), 6.76 (d, J=8.6 Hz, 1H), 6.53 (d, J=2.7 Hz, 1H), 6.46 (s, 1H), 4.50 (t, J=5.2 Hz, 1H), 3.72 (s, 3H), 3.56 (s, 2H), 3.35 (s, 6H), 2.40-2.82 (m, 10H). HR-MS (ESI): calc. for C₂₉H₃₄ClN₄O₄ ([M+H]⁺): 537.2263; found: 537.2257. RP-HPLC-UV-Purity (C18 column, 0.1% TFA in water/0.1% AA in MeCN): 98.0% (210 nm), 98.0% (254 nm). C₂₉H₃₃ClN₄O₄ (536.22).

### 4-((6-Chloro-2-methoxyacridin-9-yl)amino)-2-((4-(3,3-dimethoxypropyl) piperazin-1-yl)methyl)-phenol 6b

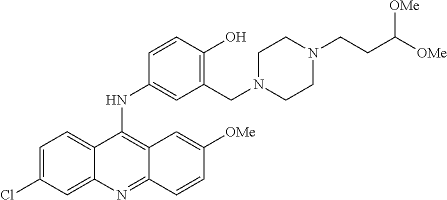 [SMILES: COc1ccc2nc3cc(Cl)ccc3c(Nc3ccc(O)c(CN4CCN(CCC(OC)OC)CC4)c3)c2c1]

The reaction was performed with 308 mg (1.11 mmol) 6,9-dichloro-2-methoxyacridine and 373 mg (1.11 mmol) 4-amino-2-((4-(3,3-diethoxy-propyl)-pi-perazin-1-yl)methyl)phenol 5b in 23 mL MeOH (in accordance with general procedure 3). Appearance: Orange solid. Yield: 342 mg, 0.62 mmol, 56%. R_{F} (CH₂Cl₂/MeOH 5:1)=0.65. IR (film): v (cm⁻¹)=3328; 2948; 2828; 1631; 1561; 1494; 1437; 1420; 1363; 1255; 1235; 1124; 1070; 1032; 1009; 925; 874; 828; 736; 702. ¹H-NMR (500 MHz, CDCl₃): δ (ppm)=8.13 (s, 1H), 8.03 (s, 1H), 7.90 (d, J=9.2 Hz, 1H), 7.41 (d, J=9.4 Hz, 1H), 7.07 (d, J=7.9 Hz, 1H), 6.85 (dd, J=8.6 Hz, J=2.7 Hz, 1H), 6.78 (d, J=8.6 Hz, 1H), 6.55 (d, J=2.7 Hz, 1H), 6.46 (s, 1H), 4.45 (d, J=5.7 Hz, 1H), 3.74 (s, 3H), 3.58 (s, 2H), 3.33 (s, 6H), 2.43 (dd, J=8.9 Hz, J=6.4 Hz, 8H), 1.86-1.70 (m, 2H), 1.27 (s, 2H). HR-MS (ESI): calc. for C₃₀H₃₆ClN₄O₄ ([M+H]⁺): 551.2420; found: 551.2418. C₃₀H₃₅ClN₄O₄ (550.23).

General Procedure 4: Deprotection for the Synthesis of Compounds 7

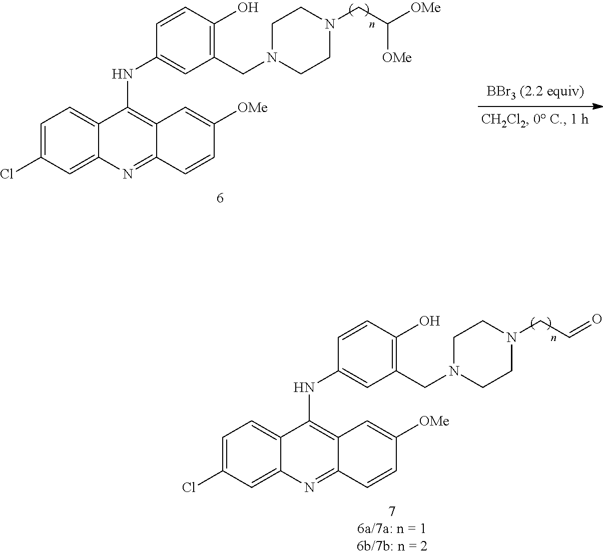

In an oven-dried flask under nitrogen atmosphere, compound 6 (1.0 equiv) was dissolved in CH₂Cl₂ (0.02 M) and cooled to 0° C. Boron tribromide (2.2 equiv, 1.0 M in CH₂Cl₂) was added dropwise, resulting in the formation of a dark red solution. The reaction mixture was stirred at 0° C. for 1 h and was warmed to rt. H₂O (1.0 mL/0.1 mmol) and a saturated NaHCO₃ solution were added (until pH=8), and the mixture was extracted with CH₂Cl₂ (3×10 mL/0.1 mmol). The combined organic phases were dried over magnesium sulfate and the solution was filtered. The solvent was removed in vacuo and the crude mixture was directly used in the next step without further purification.

### 2-(4-(5-((6-Chloro-2-methoxyacridin-9-yl)amino)-2-hydroxybenzyl)piperazin-1-yl)acetaldehyde 7a

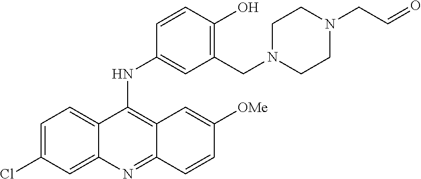 [SMILES: COc1ccc2nc3cc(Cl)ccc3c(Nc3ccc(O)c(CN4CCN(CC=O)CC4)c3)c2c1]

The reaction was performed with 322 mg (0.600 mmol) 4-((6-chloro-2-methoxyacridin-9-yl)-amino)-2-((4-(2,2-dimethoxy-ethyl)piperazin-1-yl)methyl)phenol 6a and 1.32 mL (1.32 mmol) boron tribromide in 28 mL CH₂Cl₂ (in accordance with general procedure 4). Appearance: Orange semisolid. Yield: 222 mg, 0.452 mmol, 75%. R_{F} (CH₂Cl₂/MeOH 5:1)=0.94. IR (film): v (cm⁻¹)=2937; 2828; 1729; 1631; 1561; 1494; 1420; 1364; 1302; 1254; 1235; 1158; 1135; 1031; 1009; 928; 876; 829; 735; 702. ¹H-NMR (400 MHz, CDCl₃): δ (ppm)=9.68 (s, 1H), 8.09 (s, 1H), 7.99 (d, J=9.4 Hz, 1H), 7.87 (d, J=9.2 Hz, 1H), 7.45-7.32 (m, 1H), 7.22 (d, J=9.2 Hz, 1H), 7.08 (s, 1H), 6.86 (dd, J=8.6 Hz, J=2.7 Hz, 1H), 6.77 (d, J=8.6 Hz, 1H), 6.57 (s, 1H), 3.73 (s, 3H), 3.60 (s, 2H), 3.22 (d, J=1.4 Hz, 2H), 2.89-2.20 (m, 8H). HR-MS (ESI): calc. for C₂₇H₂₈ClN₄O₃ ([M+H]⁺): 491.1844; found: 491.1852. C₂₇H₂₇ClN₄O₃ (490.18).

### 3-(4-(5-((6-Chloro-2-methoxyacridin-9-yl)amino)-2-hydroxybenzyl)piperazin-1-yl)propanal 7b

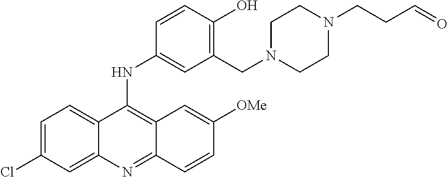 [SMILES: COc1ccc2nc3cc(Cl)ccc3c(Nc3ccc(O)c(CN4CCN(CCC=O)CC4)c3)c2c1]

The reaction was performed with 177 mg (0.312 mmol) 4-((6-chloro-2-methoxyacridin-9-yl)amino)-2-((4-(3,3-dimethoxy-propyl)piperazin-1-yl)methyl)-phenol 6b and 685 μL (0.685 mmol) boron tribromide in 13 mL CH₂Cl₂ (in accordance with general procedure 4). Appearance: Orange oil. Yield: 153 mg, 0.303 mmol, 97%. R_{F}(CH₂Cl₂/MeOH 5:1)=0.67. IR (film): v (cm⁻¹)=3255; 3060; 2925; 2852; 2830; 1723; 1631; 1605; 1561; 1518; 1494; 1467; 1438; 1421; 1364; 1343; 1302; 1254; 1234; 1136; 1071; 1032; 1003; 928; 874; 828; 778; 737; 703. ¹H-NMR (400 MHz, CDCl₃): δ (ppm)=9.76 (t, J=1.9 Hz, 1H), 8.10 (s, 1H), 7.98 (d, J=9.4 Hz, 1H), 7.83 (d, J=9.3 Hz, 1H), 7.32 (d, J=9.3 Hz, 1H), 7.17 (d, J=9.3 Hz, 1H), 7.11 (s, 1H), 6.92 (d, J=8.4 Hz, 1H), 6.78 (d, J=8.6 Hz, 1H), 6.68 (s, 1H), 3.75 (s, 3H), 3.60 (s, 2H), 2.76 (t, J=6.8 Hz, 2H), 2.59 (td, J=6.9 Hz, J=2.0 Hz, 7H), 1.25 (s, 2H), 0.87 (d, J=7.1 Hz, 1H). HR-MS (ESI): calc. for C₂₈H₃₀ClN₄O₃ ([M+H]⁺): 505.2001; found: 505.2000. C₂₈H₂₉ClN₄O₃ (504.19).

General Procedure 5: Reductive Amination for the Synthesis of Compounds 8/Gen C

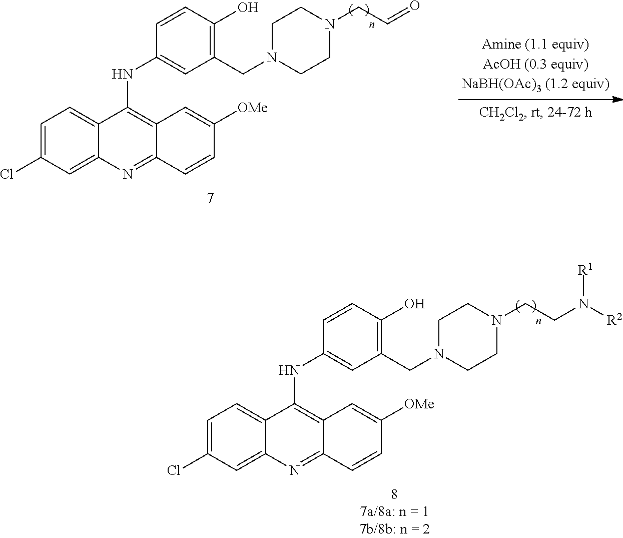

In an oven-dried flask under nitrogen atmosphere, compound 7 (1.0 equiv) was dissolved in CH₂Cl₂ (0.05 M) at rt. The amine (1.1 equiv) was added followed by the addition of glacial acetic acid (0.3 equiv) and sodium triacetoxyborohydride (1.2 equiv). After completion of the reaction, a saturated NaHCO₃ solution was added (until pH=8-9), and the mixture was extracted with CH₂Cl₂ (3×10 mL/0.1 mmol). The combined organic phases were dried over magnesium sulfate and the solution was filtered. The solvent was removed in vacuo and the residue was purified by column chromatography using CH₂Cl₂/2% NEt₃ with MeOH as gradient elution (1%→10%, depending on the polarity of compound 8). The resulting mixture was washed with H₂O (2×2.0 mL/0.1 mmol) and the combined, aqueous layers were extracted with CH₂Cl₂ (3×5.0 mL/0.1 mmol). The combined organic layers were dried over magnesium sulfate. The solution was filtered and the solvent was removed in vacuo. The resulting orange oil was additionally purified by recrystallization in n-hexane/CH₂Cl₂ (3:1, 2.0 mL/0.1 mmol) at −20° C. as well as in MeCN (2.0 mL/0.1 mmol) at −20° C.

### 4-((6-Chloro-2-methoxyacridin-9-yl)amino)-2-((4-(2-(diisopropylamino)ethyl) piperazin-1-yl)methyl)-phenol 8a/Gen C-1

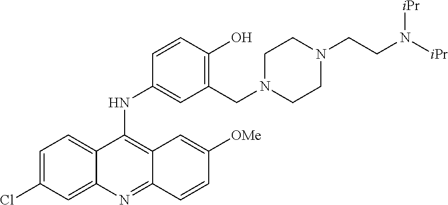 [SMILES: COc1ccc2nc3cc(Cl)ccc3c(Nc3ccc(O)c(CN4CCN(CCN(C(C)C)C(C)C)CC4)c3)c2c1]

The reaction was performed with 85.0 mg (0.173 mmol) 2-(4-(5-((6-chloro-2-methoxy-acridin-9-yl)-amino)-2-hydroxybenzyl)piperazin-1-yl)acetaldehyde 7a, 26.7 μL (19.3 mg, 0.190 mmol) diisopropylamine, 2.97 μL (3.12 mg, 520 μmol) glacial acetic acid and 44.1 mg (0.208 mmol) sodium triacetoxyborohydride in 3.5 mL CH₂Cl₂ for 72 h (in accordance with general procedure 5). Appearance: Orange solid. Yield: 33.5 mg, 0.0582 mmol, 34%. R_{F} (CH₂Cl₂/MeOH 5:1)=0.19. IR (film): v (cm⁻¹)=3227; 2964; 2877; 2820; 1631; 1606; 1562; 1519; 1494; 1437; 1420; 1362; 1333; 1303; 1254; 1235; 1150; 1132; 1072; 1032; 1008; 928; 875; 829; 803; 771; 728; 702. ¹H-NMR (500 MHz, CDCl₃): δ (ppm)=8.15 (d, J=2.1 Hz, 1H), 8.05 (d, J=9.4 Hz, 1H), 7.90 (dd, J=9.4 Hz, J=1.9 Hz, 1H), 7.42 (dd, J=9.4 Hz, J=2.7 Hz, 1H), 7.07 (t, J=2.7 Hz, 1H), 6.85 (dd, J=8.7 Hz, J=2.6 Hz, 1H), 6.77 (d, J=8.6 Hz, 1H), 6.55 (d, J=2.7 Hz, 1H), 6.45 (s, 1H), 3.75 (s, 3H), 3.58 (s, 2H), 2.99 (h, J=6.5 Hz, 2H), 2.72-2.47 (m, 8H), 2.46-2.34 (m, 2H), 1.00 (d, J=6.5 Hz, 12H). HR-MS (ESI): calc. for C₃₃H₄₃ClN₅O₂ ([M+H]⁺): 576.3100; found: 576.3084. RP-HPLC-UV-Purity (C18 column, 0.1% TFA in H₂O/0.1% AA in MeCN): 98.4% (210 nm), 98.9% (254 nm). C₃₃H₄₂ClN₅O₂ (575.30).

### 4-((6-Chloro-2-methoxyacridin-9-yl)amino)-2-((4-(3-((2,3-dihydro-1H-inden-2-yl)(methyl)amino)-propyl)piperazin-1-yl)methyl)phenol 8b/Gen C-3 (TFA)

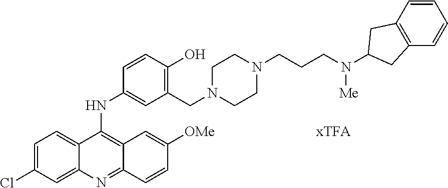 [SMILES: COc1ccc2nc3cc(Cl)ccc3c(Nc3ccc(O)c(CN4CCN(CCCN(C)C5Cc6ccccc6C5)CC4)c3)c2c1]

The reaction was performed with 143 mg (0.284 mmol) 3-(4-(5-((6-chloro-2-methoxy-acridin-9-yl)amino)-2-hydroxybenzyl)-piperazin-1-yl)-propanal 7b, 45.9 mg (0.311 mmol)N-methyl-2,3-dihydro-1H-inden-2-amine, 4.86 μL (5.01 mg, 850 μmol) glacial acetic acid and 72.0 mg (0.340 mmol) sodium triacetoxyborohydride in 5.7 mL CH₂Cl₂ for 24 h (in accordance with general procedure 5). Appearance: Orange semisolid. Yield: 31.1 mg, 0.0490 mmol, 17%. R_{F} (CH₂Cl₂/MeOH 5:1)=0.45. IR (film): v (cm⁻¹)=3179; 3049; 2925; 2856; 1672; 1587; 1560; 1445; 1207; 1138; 1030; 938; 844; 803; 763; 726; 679. ¹H-NMR (500 MHz, CD₃OD): δ (ppm)=8.06 (d, J=9.4 Hz, 1H), 7.92-7.83 (m, 2H), 7.68 (dd, J=9.3 Hz, J=2.5 Hz, 1H), 7.63 (d, J=2.5 Hz, 1H), 7.58 (d, J=7.7 Hz, 1H), 7.45-7.36 (m, 4H), 7.36-7.30 (m, 2H), 7.10 (d, J=8.5 Hz, 1H), 5.11 (dd, J=8.6 Hz, J=3.3 Hz, 1H), 3.78 (s, 3H), 3.34 (s, 1H), 3.24-3.07 (m, 4H), 2.76-2.62 (m, 4H), 2.63-2.47 (m, 2H), 2.42 (ddt, J=15.4 Hz, J=8.1 Hz, J=3.7 Hz, 1H), 2.01 (dd, J=16.7 Hz, J=7.8 Hz, 2H), 1.44-1.24 (m, 2H), 1.16 (ddd, J=19.2 Hz, J=17.3 Hz, J=6.7 Hz, 2H), 0.98-0.82 (m, 2H). HR-MS (ESI): calc. for C₃₈H₄₃ClN₅O₂ ([M+H]⁺): 636.3100; found: 636.3096. RP-HPLC-UV-Purity (C18 column, 0.1% TFA in H₂O/0.1% AA in MeCN): 69.9% (210 nm), 67.5% (254 nm). Additional purification by semipreparative HPLC (C8 column): RP-HPLC-UV-Purity (C3 column, 0.1% TFA in H₂O/0.1% AA in MeCN): F1: 98.3% (210 nm), 97.8% (254 nm) and F2: 96.8% (210 nm), 95.4% (254 nm). C₃₈H₄₂ClN₅O₂ (635.30).

### 4-((6-Chloro-2-methoxyacridin-9-yl)amino)-2-((4-(2-(phenylamino)ethyl) piperazin-1-yl)methyl)-phenol 8c/Gen C-5

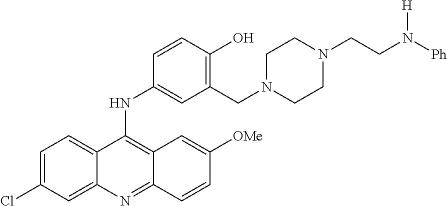 [SMILES: COc1ccc2nc3cc(Cl)ccc3c(Nc3ccc(O)c(CN4CCN(CCNc5ccccc5)CC4)c3)c2c1]

The reaction was performed with 71.6 mg (0.146 mmol) 2-(4-(5-((6-chloro-2-methoxy-acridin-9-yl)-amino)-2-hydroxybenzyl)piperazin-1-yl)acetaldehyde-7a, 14.7 μL (15.0 mg, 0.161 mmol) aniline, 2.51 μL (2.63 mg, 438 μmol) glacial acetic acid and 37.2 mg (0.175 mmol) sodium triacetoxyborohydride in 2.9 mL CH₂Cl₂ for 48 h (in accordance with general procedure 5). Appearance: Orange solid. Yield: 40.8 mg, 0.0719 mmol, 49%. R_{F}(CH₂Cl₂/MeOH 2:1)=0.96. IR (film): v (cm⁻¹)=3372; 3049; 2944; 2874; 2826; 1923; 1631; 1603; 1560; 1493; 1421; 1362; 1344; 1305; 1255; 1235; 1179; 1153; 1071; 1031; 1010; 992; 929; 872; 828; 751; 736; 694. ¹H-NMR (500 MHz, CDCl₃): δ (ppm)=8.15 (s, 1H), 8.06 (d, J=9.4 Hz, 1H), 7.91 (d, J=9.3 Hz, 1H), 7.42 (d, J=9.3 Hz, 1H), 7.24-7.12 (m, 1H), 7.07 (d, J=5.8 Hz, 1H), 6.85 (dd, J=8.6 Hz, J=2.7 Hz, 1H), 6.78 (d, J=8.6 Hz, 2H), 6.72 (tt, J=7.3 Hz, J=1.1 Hz, 1H), 6.67-6.58 (m, 2H), 6.55 (d, J=2.7 Hz, 1H), 6.45 (s, 1H), 3.75 (s, 3H), 3.60 (s, 2H), 3.50 (s, 2H), 3.17 (t, J=5.9 Hz, 2H), 2.90-2.27 (m, 8H). HR-MS (ESI): calc. for C₃₃H₃₅ClN₅O₂ ([M+H]⁺): 568.2474; found: 568.2471. RP-HPLC-UV-Purity (C18 column, 0.1% TFA in H₂O/0.1% AA in MeCN): 97.2% (210 nm), 97.1% (254 nm). C₃₃H₃₄ClN₅O₂ (567.24).

### 4-((6-Chloro-2-methoxyacridin-9-yl)amino)-2-((4-(2-((thiophen-2-ylmethyl) amino)ethyl)piperazin-1-yl)methyl)phenol 8d/Gen C-6 (TFA)

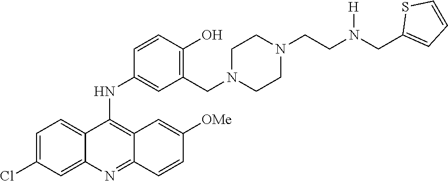 [SMILES: COc1ccc2nc3cc(Cl)ccc3c(Nc3ccc(O)c(CN4CCN(CCNCc5cccs5)CC4)c3)c2c1]

The reaction was performed with 85.0 mg (0.173 mmol) 2-(4-(5-((6-chloro-2-methoxy-acridin-9-yl)amino)-2-hydroxybenzyl)piperazin-1-yl)acet-aldehyde 7a, 26.7 μL (19.3 mg, 0.190 mmol) thiophen-2-ylmethanamine, 2.97 μL (3.12 mg, 520 μmol) glacial acetic acid and 44.1 mg (0.208 mmol) sodium triacetoxyborohydride in 3.0 mL CH₂Cl₂ for 72 h (in accordance with general procedure 5). Appearance: Orange semisolid. Yield: 9.1 mg, 0.0156 mmol, 9%. R_{F} (CH₂Cl₂/MeOH 5:1)=0.64. IR (film): v (cm⁻¹)=3352; 2925; 2853; 1680; 1587; 1561; 1496; 1443; 1377; 1209; 1181; 1137; 1088; 1035; 845; 802; 725. ¹H-NMR (400 MHz, CD₃OD): δ (ppm)=8.11 (s, 1H), 8.01 (s, 2H), 7.88 (d, J=9.2 Hz, 1H), 7.39 (d, J=9.4 Hz, 2H), 7.20 (dd, J=5.0 Hz, J=1.3 Hz, 1H), 7.07 (s, 1H), 6.98-6.89 (m, 2H), 6.88-6.82 (m, 1H), 6.76 (d, J=8.6 Hz, 1H), 6.55 (s, 1H), 4.00 (s, 2H), 3.73 (s, 3H), 3.57 (s, 2H), 2.72 (t, J=6.0 Hz, 2H), 2.66-2.33 (m, 8H), 2.17 (s, 2H). HR-MS (ESI): calc. for C₃₂H₃₃ClN₅O₂S ([M−H]⁻): 586.2049; found: 586.2053. HR-MS (ESI): calc. for C₃₂H₃₅ClN₅O₂S ([M+H]⁺): 588.2195; found: 588.2191. RP-HPLC-UV-Purity (C18 column, 0.1% TFA in H₂O/0.1% AA in MeCN): 91.3% (210 nm), 89.0% (254 nm). Additional purification by semipreparative HPLC (C8 column): RP-HPLC-UV-Purity (C3 column, 0.1% TFA in H₂O/0.1% AA in MeCN): F1: 100% (210 nm)/100% (254 nm) and F2: 97.6% (210 nm)/99.7% (254 nm). C₃₂H₃₄ClN₅O₂S (587.21).

### 2-((4-(2-(Benzyl(methyl)amino)ethyl)piperazin-1-yl)methyl)-4-((6-chloro-2-methoxyacridin-9-yl)-amino)phenol 8e/Gen C-9

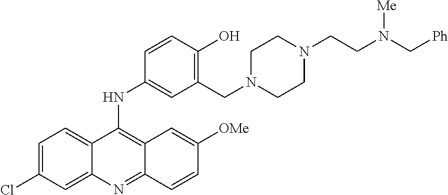 [SMILES: COc1ccc2nc3cc(Cl)ccc3c(Nc3ccc(O)c(CN4CCN(CCN(C)Cc5ccccc5)CC4)c3)c2c1]

The reaction was performed with 72.5 mg (0.148 mmol) 2-(4-(5-((6-chloro-2-methoxy-acridin-9-yl)amino)-2-hydroxybenzyl)piperazin-1-yl)acet-aldehyde 7a, 21.0 μL (19.7 mg, 0.163 mmol) methylbenzylamine, 2.53 μL (2.67 mg, 444 μmol) glacial acetic acid and 37.6 mg (0.178 mmol) sodium triacetoxyborohydride in 2.1 mL CH₂Cl₂ for 48 h (in accordance with general procedure 5). Appearance: Yellow-orange solid. Yield: 29.5 mg, 0.0496 mmol, 34%. R_{F} (CH₂Cl₂/MeOH 5:1)=0.61. IR (film): v (cm⁻¹)=3062; 3023; 2945; 2818; 1631; 1561; 1518; 1494; 1437; 1420; 1364; 1254; 1234; 1155; 1072; 1030; 1008; 928; 874; 829; 778; 738; 700. ¹H-NMR (400 MHz, CDCl₃): δ (ppm)=8.14 (s, 1H), 8.03 (s, 1H), 7.89 (d, J=9.6 Hz, 1H), 7.41 (d, J=9.7 Hz, 1H), 7.30 (d, J=4.3 Hz, 3H), 7.25-7.20 (m, 2H), 7.06 (s, 1H), 6.83 (dd, J=8.6 Hz, J=2.7 Hz, 1H), 6.76 (d, J=8.5 Hz, 1H), 6.54 (d, J=2.7 Hz, 1H), 6.44 (s, 1H), 3.74 (s, 3H), 3.57 (s, 2H), 3.51 (s, 2H), 2.85-2.32 (m, 10H), 2.24 (s, 3H), 1.26 (s, 2H). HR-MS (ESI): calc. for C₃₅H₃₉ClN₅O₂ ([M+H]⁺): 596.2787; found: 596.2770. RP-HPLC-UV-Purity (C18 column, 0.1% TFA in H₂O/0.1% AA in MeCN): 99.7% (210 nm), 99.7% (254 nm). C₃₅H₃₈ClN₅O₂ (595.27).

Three-Step Synthesis of 3^{rd} Generation Compound 8a/Gen C-1

General Procedure 6: Multi-Component Reaction for the Synthesis of Compounds 10

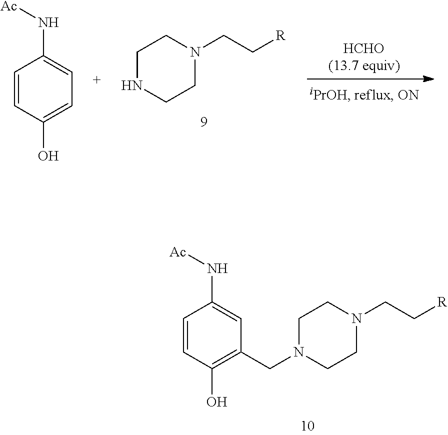

Acetaminophen (1.0 equiv) was dissolved in iPrOH (0.1 M) and formaldehyde (13.7 equiv) was added, followed by the addition of compound 9 (1.5 equiv). The reaction mixture was heated to 70° C. for 24 h. The reaction was cooled down to rt and the solvent was removed in vacuo. The mixture was partially purified by column chromatography using CH₂Cl₂/2% NEt₃ with MeOH as gradient elution (2%→10%). The compounds were used in the next step without an additional purification.

### N-(3-((4-(2-(Diisopropylamino)ethyl)piperazin-1-yl)methyl)-4-hydroxy phenyl)acetamide 10a

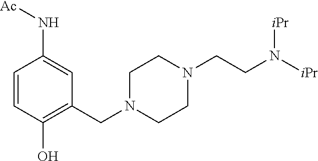 [SMILES: CC(=O)Nc1ccc(O)c(CN2CCN(CCN(C(C)C)C(C)C)CC2)c1]

The reaction was performed with 98.1 mg (0.649 mmol) acetaminophen, 260 μL (267 mg, 8.89 mmol) formaldehyde and 208 mg (0.976 mmol)N-isopropyl-N-(2-(piperazin-1-yl)ethyl)-propan-2-amine 9a in 6.5 mL iPrOH (in accordance with general procedure 6). Appearance: Light yellow oil. Yield: 214 mg, 0.568 mmol, 88%. R_{F}(CH₂Cl₂/MeOH 5:1)=0.30. C₂₁H₃₆N₄O₂ (376.28).

General Procedure 7: Acetal-Deprotection for the Synthesis of Compounds 11

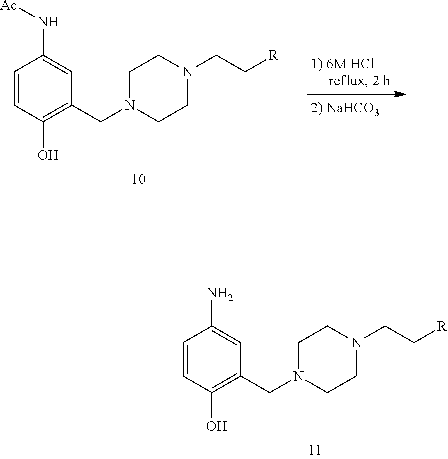

Compound 10 (1.0 equiv) and a 6 N HCl solution (0.4 M) were heated to 100° C. for 2 h. The mixture was cooled down to rt, a saturated NaHCO₃ solution (until pH=8-9) and CH₂Cl₂ were added (10 mL/0.1 mmol). The mixture was stirred at rt for 30 min. The phases were separated and the aqueous layer was extracted with CH₂Cl₂ (3×10 mL/0.1 mmol). The combined organic layers were dried over magnesium sulfate, the solution was filtered and the solvent was removed in vacuo.

### 4-Amino-2-((4-(2-(diisopropylamino)ethyl)piperazin-1-yl)methyl)phenol 11a

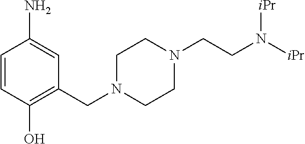 [SMILES: CC(C)N(CCN1CCN(Cc2cc(N)ccc2O)CC1)C(C)C]

The reaction was performed with 571 mg (1.52 mmol)N-(3-((4-(2-(diisopropylamino)ethyl)piperazin-1-yl)methyl)-4-hydroxyphenyl)-acetamide 10a in 3.8 mL 6 M HCl (in accordance with general procedure 7). Appearance: Light yellow oil. Yield: 369 mg, 1.10 mmol, 73%. R_{F}(CH₂Cl₂/MeOH 5:1)=0.17. IR (film): v (cm⁻¹)=3426; 3349; 3208; 2963; 2874; 2819; 1631; 1498; 1458; 1380; 1361; 1347; 1334; 1313; 1289; 1245; 1219; 1206; 1151; 1133; 1080; 1007; 931; 866; 814; 774; 744. ¹H NMR (400 MHz, CDCl₃): δ (ppm)=6.65 (d, J=8.4 Hz, 1H), 6.55 (dd, J=8.4 Hz, J=2.8 Hz, 1H), 6.38 (d, J=2.8 Hz, 1H), 3.60 (s, 2H), 3.11-2.90 (m, 2H), 2.88-2.80 (m, 2H), 2.75-2.30 (m, 12H), 0.99 (d, J=6.5 Hz, 12H). HR-MS (ESI): calc. for C₁₉H₃₅N40 ([M+H]⁺): 335.2805; found: 335.2807. C₁₉H₃₄N₄O (334.27).

General Procedure 8: Nucleophilic Aromatic Substitution for the Synthesis of Compounds 8

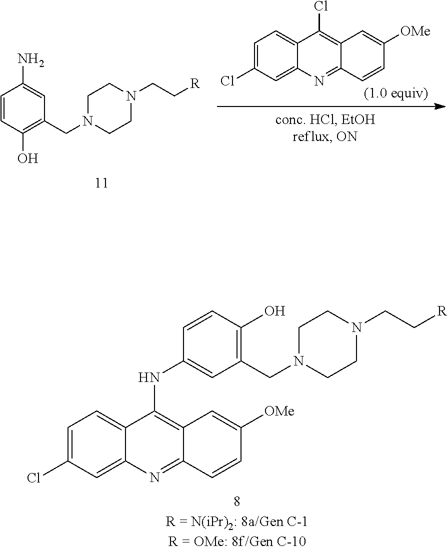

6,9-Dichloro-2-methoxyacridine (1.0 equiv) and compound 11 (1.0 equiv) were dissolved in EtOH (0.05 M). Subsequently, conc. HCl (1 drop/0.5 mmol) was added to the yellow suspension, which resulted in the formation of an orange solution. The reaction mixture was heated to 80° C. for 24 h. After cooling down, a saturated NaHCO₃ solution was added (until pH=8-9), and the mixture was extracted with CH₂Cl₂ (3×50 mL/1.0 mmol). The combined organic phases were dried over magnesium sulfate. The solution was filtered and the solvent was removed in vacuo. The residue was purified by column chromatography using CH₂Cl₂/2% NEt₃ with MeOH as gradient elution (1%→5%).

### Synthesis of 4-((6-Chloro-2-methoxyacridin-9-yl)amino)-2-((4-(2-(diisopropylamino)-ethyl)piperazin-1-yl)methyl)-phenol 8a/Gen C-1

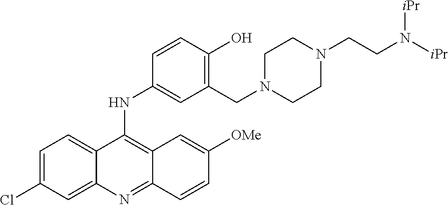 [SMILES: COc1ccc2nc3cc(Cl)ccc3c(Nc3ccc(O)c(CN4CCN(CCN(C(C)C)C(C)C)CC4)c3)c2c1]

The reaction was performed with 79.8 mg (0.287 mmol) 6,9-dichloro-2-methoxyacridine and 95.9 mg (0.287 mmol) 4-amino-2-((4-(2-(diisopropyl-amino)ethyl)piperazin-1-yl)methyl)phenol 10 in 5.5 mL EtOH (in accordance with general procedure 8). Appearance: Orange solid. Yield: 107 mg, 0.186 mmol, 65%. RP-HPLC-UV-Purity (C18 column, 0.1% TFA in H₂O/0.1% AA in MeCN): F1: 99.0% (210 nm), 99.5% (254 nm); F2: 100% (210 nm), 99.7% (254 nm) and F3: 97.3% (210 nm), 96.3% (254 nm). C₃₃H₄₂ClN₅O₂ (575.30).

Synthesis of 3^{rd} Generation Compound 8f/Gen C-7

### Synthesis of tert-Butyl-4-(2-hydroxy-5-nitrobenzyl)piperazine-1-carboxylate 12

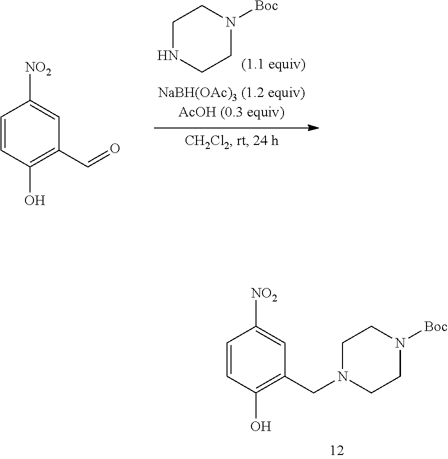 [SMILES: O=Cc1cc([N+](=O)[O-])ccc1O ; CC(C)(C)OC(=O)N1CCNCC1 ; CC(C)(C)OC(=O)N1CCN(Cc2cc([N+](=O)[O-])ccc2O)CC1]

In an oven-dried flask under nitrogen atmosphere, 2-nitro-5-benzaldehyde (202 mg, 1.21 mmol, 1.0 equiv) was dissolved in 5.0 mL CH₂Cl₂ at rt. tert-Butyl-piperazine-1-carboxylate (248 mg, 1.33 mmol, 1.1 equiv) was added followed by the addition of glacial acetic acid (20.8 μL, 21.8 mg, 0.363 mmol, 0.3 equiv) and sodium triacetoxy-borohydride (308 mg, 1.45 mmol, 1.2 equiv). After stirring at rt ON, a saturated NaHCO₃ solution was added (until pH=8-9), and the mixture was extracted with CH₂Cl₂ (3×100 mL). The combined organic phases were dried over magnesium sulfate and the solution was filtered. The solvent was removed in vacuo and the residue was purified by column chromatography using CH₂Cl₂/2% NEt₃ with MeOH as gradient elution (1%→10%). 362 mg (1.07 mmol, 89%) of compound 12 were isolated as a light yellow solid.

R_{F} (CH₂Cl₂/MeOH 5:1)=0.83. IR (film): v (cm⁻¹)=2977; 2930; 2856; 1694; 1620; 1589; 1523; 1478; 1421; 1366; 1337; 1286; 1248; 1170; 1141; 1088; 999; 903; 865; 798; 771; 753. ¹H NMR (400 MHz, CDCl₃): δ (ppm)=8.10 (dd, J=9.0 Hz, J=2.8 Hz, 1H), 7.95 (d, J=2.7 Hz, 1H), 6.87 (d, J=9.0 Hz, 1H), 3.80 (s, 2H), 3.66-3.24 (m 4H), 2.77-2.40 (m, 4H), 1.46 (s, 9H). HR-MS (ESI): calc. for C₁₆H₂₂N₃O₅ ([M−H]⁻): 336.1565; found: 336.1565. C₁₆H₂₃N₃O₅ (337.16).

### Synthesis of 4-Nitro-2-(piperazin-1-ylmethyl)Phenol 13

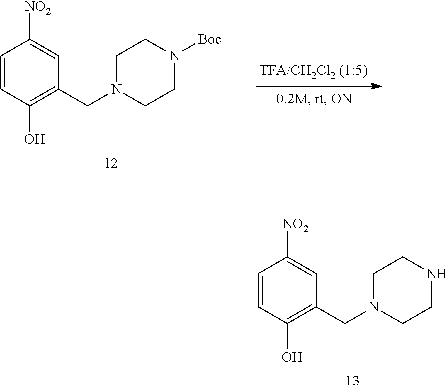 [SMILES: CC(C)(C)OC(=O)N1CCN(Cc2cc([N+](=O)[O-])ccc2O)CC1 ; O=[N+]([O-])c1ccc(O)c(CN2CCNCC2)c1]

tert-Butyl-4-(2-hydroxy-5-nitrobenzyl)piperazine-1-carboxylate 12 (708 mg, 2.10 mmol, 1.0 equiv) dissolved in 8.75 mL CH₂Cl₂ (0.2 M) at rt. 1.75 mL Trifluoroacetic acid (1:5 TFA/CH₂Cl₂) were added and the reaction mixture was stirred at rt ON. After completion of the reaction, a saturated NaHCO₃ solution was added (until pH=8-9), and the mixture was extracted with CH₂Cl₂ (3×50 mL). The combined organic phases were dried over magnesium sulfate. The solution was filtered and the solvent was removed in vacuo. 309 mg (1.31 mmol, 62%) of compound 13 (also commercially available) were isolated as a yellow solid.

R_{F} (CH₂Cl₂/MeOH 5:1)=0.39. IR (film): v (cm⁻¹)=2949; 2823; 1590; 1482; 1336; 1279; 1162; 1127; 1088; 998; 932; 835; 793; 754; 734; 701. ¹H NMR (400 MHz, CDCl₃): δ (ppm)=8.10 (dd, J=9.0 Hz, J=2.7 Hz, 1H), 7.95 (d, J=2.7 Hz, 1H), 6.85 (d, J=9.0 Hz, 1H), 3.79 (s, 2H), 2.97 (s, 2H), 2.77-2.42 (m, 4H). HR-MS (ESI): calc. for C₁₁H₁₆N₃O₃ ([M+H]⁺): 238.1186; found: 238.1184. C₁₁H₁₅N₃O₃ (237.11).

### Synthesis of Methyl-2,2-dimethyl-3-oxopropanoate 14

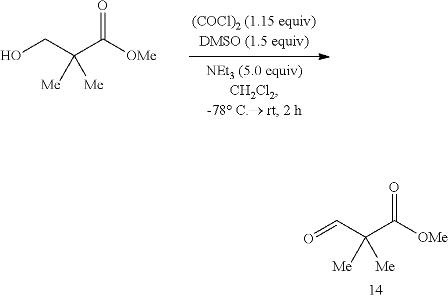 [SMILES: COC(=O)C(C)(C)CO ; COC(=O)C(C)(C)C=O]

According to the procedure of: B. M. Trost, M. J. Bartlett, A. H. Weiss, A. Jacobi von Wangelin, V. S. Chan, Chem. Eur. J. 2012, 18, 16498-16509. In an oven-dried flask under nitrogen atmosphere, oxalyl chloride (1.56 mL, 2.24 g, 17.6 mmol, 1.15 equiv) was dissolved in 37 mL CH₂Cl₂ and cooled to −78° C. A solution of DMSO (1.63 mL, 1.80 g, 23.1 mmol, 1.5 equiv) in 2.0 mL CH₂Cl₂ was added over a period of 20 min, followed by the addition of 2.03 g (15.3 mmol, 1.0 equiv) methyl-3-hydroxy-2,2-dimethylpropanoate (in 8.0 mL CH₂Cl₂) over a period of 15 min. The mixture was stirred at −78° C. for 15 min and triethylamine (10.6 mL, 7.76 g, 76.7 mmol, 5.0 equiv) was added dropwise. The reaction was allowed to warm rt and 10 mL H₂O was added. The phases were separated and the aqueous layer was extracted with EtOAc (3×100 mL). The combined organic layers were dried over magnesium sulfate. The solution was filtered and the solvent was removed in vacuo. 698 mg (5.37 mmol, 35%) of compound 14 were isolated as a colourless oil.

IR (film): v (cm⁻¹)=3450; 2980; 2954; 2876; 1854; 1804; 1733; 1476; 1435; 1368; 1273; 1195; 1158; 1093; 988; 870; 819; 769; 744. ¹H NMR (500 MHz, CDCl₃): δ (ppm)=9.67 (s, 1H), 3.77 (s, 3H), 1.37 (s, 6H). HR-MS (E1): m/z (%)=calc. for C₆H₁₁O₃ 131.0710; found: 131.0710 (17.3); calc. for CH₁₀O₂: 102.0681; found: 102.0681 (100); calc. for C₃H₇O: 59.0497; found: 59.0495 (37.0). C₆H₁₀O₃ (130.06).

### Synthesis of Methyl-3-(4-(2-hydroxy-5-nitrobenzyl)piperazin-1-yl)-2,2-dimethyl-pro-panoate 15

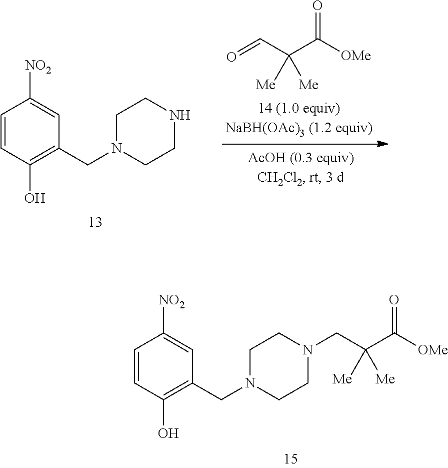 [SMILES: O=[N+]([O-])c1ccc(O)c(CN2CCNCC2)c1 ; COC(=O)C(C)(C)C=O ; COC(=O)C(C)(C)CN1CCN(Cc2cc([N+](=O)[O-])ccc2O)CC1]

In an oven-dried flask under nitrogen atmosphere, 231 mg (0.975 mmol, 1.0 equiv) 4-nitro-2-(piperazin-1-ylmethyl)phenol 13 were dissolved in 5.0 mL CH₂Cl₂ at rt. 127 mg (0.975 mmol, 1.0 equiv) methyl-2,2-dimethyl-3-oxopropanoate 14 were added dropwise, followed by the addition of glacial acetic acid (1.67 μL, 1.76 mg, 0.0293 mmol, 0.3 equiv) and sodium triacetoxyborohydride (248 mg, 0.975 mmol, 1.2 equiv). After stirring at rt ON, a saturated NaHCO₃ solution was added (until pH=8-9), and the mixture was extracted with CH₂Cl₂ (3×20 mL). The combined organic phases were dried over magnesium sulfate and the solution was filtered. The solvent was removed in vacuo and the residue was purified by column chromatography using CH₂Cl₂/2% NEt₃/1% MeOH. 204 mg (0.580 mmol, 60%) of compound 15 were isolated as a light yellow solid.

R_{F} (CH₂Cl₂/MeOH 5:1)=0.96. IR (film): v (cm⁻¹)=2950; 2832; 1728; 1620; 1590; 1523; 1479; 1461; 1338; 1283; 1191; 1156; 1122; 1088; 1009; 930; 903; 866; 825; 793; 771; 753; 720; 660. ¹H NMR (500 MHz, CDCl₃): δ (ppm)=8.10 (dd, J=9.0 Hz, J=2.8 Hz, 1H), 7.94 (dd, J=2.8 Hz, J=0.9 Hz, 1H), 6.85 (d, J=9.0 Hz, 1H), 3.77 (s, 2H), 3.67 (s, 3H), 2.75 (d, J=7.2 Hz, 2H), 2.67-2.45 (m, 8H), 1.18 (s, 6H). HR-MS (ESI): calc. for C₁₇H₂₆N305 ([M+H]⁺): 352.1867; found: 352.1864. C₁₇H₂₅N₃O₅ (351.18).

### Synthesis of 3-(4-(2-Hydroxy-5-nitrobenzyl)piperazin-1-yl)-N-2,2-trimethylpropan-amide 18

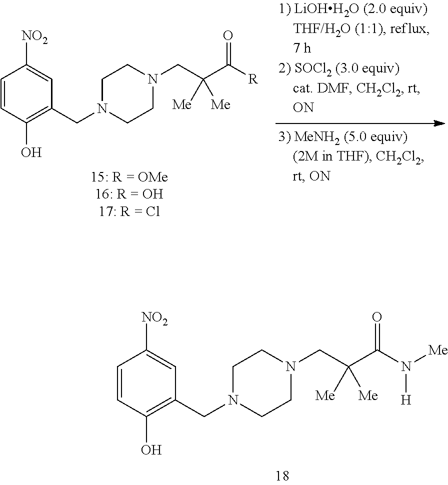

515 mg (1.47 mmol, 1.0 equiv) methyl-3-(4-(2-hydroxy-5-nitrobenzyl)piperazin-1-yl)-2,2-dimethyl-propanoate 15 were dissolved in 3.7 mL THF/H₂O (1:1). LiOH monohydrate (123 mg, 2.93 mmol, 2.0 equiv) was added and the mixture was heated to 90° C. for 7 h. The reaction was cooled down to rt and was extracted with CH₂Cl₂ (2×10 mL) to remove unreacted starting material 15. A saturated NaHCO₃ solution was added (until pH=8-9), and the mixture was extracted with CH₂Cl₂ (3×30 mL). The combined organic phases were dried over magnesium sulfate. The solution was filtered and the solvent was removed in vacuo. 280 mg (0.830 mmol, 57%) of compound 16 were isolated as a colourless solid and were directly used in the next step without further purification.

In an oven-dried flask under nitrogen atmosphere, 263 mg (0.780 mmol, 1.0 equiv) 3-(4-(2-hydroxy-5-nitrobenzyl)piperazin-1-yl)-2,2-dimethyl-propanoic acid 16 were dissolved in 7.0 mL CH₂Cl₂. Five drops of DMF were added, followed by the dropwise addition of thionyl chloride (170 μL, 279 mg, 2.34 mmol, 3.0 equiv). The reaction mixture was stirred at rt ON and the solvent was removed in vacuo. 277 mg (0.780 mmol, quant.) of compound 17 were isolated as a beige solid and was directly used in the next step without further purification.

In an oven-dried flask under nitrogen atmosphere, 277 mg (0.780 mmol, 1.0 equiv) 3-(4-(2-hydroxy-5-nitrobenzyl)piperazin-1-yl)-2,2-dimethyl-propanoyl chloride 17 were dissolved in 20 mL CH₂Cl₂. Methylamine (1.95 mL, 2 M in THF, 5.0 equiv) was added dropwise to the suspension, resulting in the formation of a yellow solution. The mixture was stirred at rt ON and 10 mL H₂O were added. The phases were separated and the aqueous layer was extracted with CH₂Cl₂ (3×50 mL). The combined organic phases were dried over magnesium sulfate and the solution was filtered. The solvent was removed in vacuo and the residue was purified by column chromatography using CH₂Cl₂/2% NEt₃ with MeOH as gradient elution (1%→3%). 236 mg (0.674 mmol, 86%) of compound 18 were isolated as a light yellow solid.

R_{F} (CH₂Cl₂/MeOH 5:1)=0.94. IR (film): v (cm⁻¹)=3350; 2939; 2824; 1652; 1621; 1590; 1526; 1480; 1460; 1408; 1337; 1284; 1155; 1123; 1087; 1008; 930; 902; 827; 792; 771; 754; 735; 661. ¹H-NMR (400 MHz, CDCl₃): δ (ppm)=8.10 (dd, J=9.0 Hz, J=2.8 Hz, 1H), 7.94 (d, J=2.8 Hz, 1H), 6.86 (d, J=9.0 Hz, 1H), 3.78 (s, 2H), 3.09-2.84 (m, 2H), 2.79 (d, J=4.8 Hz, 3H), 2.72-2.51 (m, 4H), 2.45 (s, 2H), 1.27 (d, J=8.6 Hz, 4H), 1.15 (s, 6H). HR-MS (ESI): calc. for C₁₇H₂₇N₄O₄ ([M+H]⁺): 351.2027; found: 351.2028. C₁₇H₂₆N₄O₄ (350.20).

### Synthesis of 3-(4-(5-Amino-2-hydroxybenzyl)piperazin-1-yl)-N-2,2-trimethylpropan-amide 19

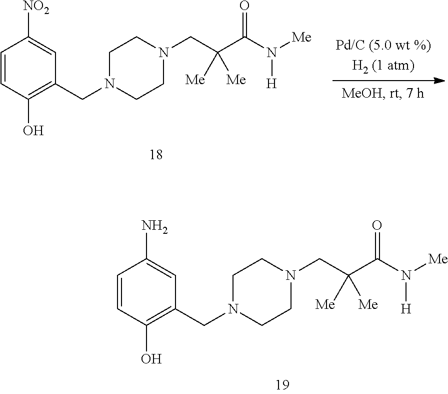 [SMILES: CNC(=O)C(C)(C)CN1CCN(Cc2cc([N+](=O)[O-])ccc2O)CC1 ; CNC(=O)C(C)(C)CN1CCN(Cc2cc(N)ccc2O)CC1]

In an oven-dried flask under nitrogen atmosphere, 50 mg Pd/C (5.0 wt %) were suspended in 10 mL MeOH and 148 mg (0.422 mmol, 1.0 equiv) 3(-(4-(2-hydroxy-5-nitrobenzyl)piperazin-1-yl)-N-2,2-trimethylpropanamide 18 were added. The resulting dark suspension was stirred under 1 atm for 7 h. Subsequently, the crude mixture was filtered over Celite® and washed with 50 mL MeOH. The solvent was removed in vacuo and the residue was purified by column chromatography using CH₂Cl₂/2% NEt₃ with MeOH as gradient elution (1%→5%). 77.1 mg (0.241 mmol, 57%) of compound 19 were isolated as a light yellow oil.

R_{F} (CH₂Cl₂/MeOH 5:1)=0.69. IR (film): v (cm⁻¹)=3344; 3234; 3049; 2949; 2877; 2820; 1648; 1538; 1498; 1457; 1408; 1381; 1346; 1316; 1288; 1245; 1221; 1154; 1123; 1009; 934; 870; 818; 773; 735; 701. ¹H NMR (400 MHz, CDCl₃): δ (ppm)=6.65 (d, J=8.5 Hz, 1H), 6.55 (dd, J=8.5 Hz, J=2.8 Hz, 1H), 6.37 (d, J=2.8 Hz, 1H), 3.60 (s, 2H), 2.77 (d, J=4.8 Hz, 3H), 2.57 (d, J=44.1 Hz, 6H), 2.41 (s, 2H), 1.32-1.17 (m, 2H), 1.13 (s, 6H). HR-MS (ESI): calc. for C₁₇H₂₉N₄O₂ ([M+H]⁺): 321.2285; found: 321.2284. C₁₇H₂₈N₄O₂ (320.22).

### Synthesis of 3-(4-(5-((6-Chloro-2-methoxyacridin-9-yl)amino)-2-hydroxybenzyl)-piperazin-1-yl)-N-2,2-trimethylpropanamide 8f/Gen C-7

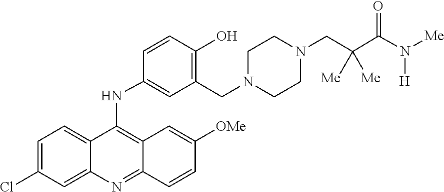 [SMILES: CNC(=O)C(C)(C)CN1CCN(Cc2cc(Nc3c4ccc(Cl)cc4nc4ccc(OC)cc34)ccc2O)CC1]

The reaction was performed with 59.2 mg (0.213 mmol) 6,9-dichloro-2-methoxyacridine and 68.1 mg (0.213 mmol) 3-(4-(5-amino-2-hydroxy-benzyl)piperazin-1-yl)-N-2,2-trimethylpropan-amide 19 in 4.5 mL EtOH (in accordance with general procedure 8). Appearance: Orange solid. Yield: 41.4 mg, 0.0738 mmol, 35%. R_{F}(CH₂Cl₂/MeOH 5:1)=0.42. IR (film): v (cm⁻¹)=3276; 2958; 2826; 1631; 1561; 1520; 1494; 1438; 1418; 1363; 1253; 1233; 1153; 1120; 1083; 1070; 1031; 1009; 928; 873; 829; 803; 777; 736; 702; 665. ¹H-NMR (400 MHz, CDCl₃): δ (ppm)=8.09 (s, 1H), 8.01 (s, 1H), 7.87 (d, J=9.3 Hz, 1H), 7.38 (d, J=9.4 Hz, 1H), 7.25-7.13 (m, 1H), 7.07 (s, 1H), 6.86 (dd, J=8.6 Hz, J=2.7 Hz, 1H), 6.77 (d, J=8.6 Hz, 1H), 6.49 (d, J=2.6 Hz, 1H), 3.72 (s, 3H), 3.55 (s, 2H), 2.75 (d, J=4.8 Hz, 2H), 2.57 (d, J=40.5 Hz, 4H), 2.41 (s, 2H), 1.25 (s, 2H), 1.12 (s, 6H). HR-MS (ESI): calc. for C₃₁H₃₇ClN₅O₃ ([M+H]⁺): 562.2579; found: 562.2571. RP-HPLC-UV-Purity (C18 column, 0.1% TFA in H₂O/0.1% AA in MeCN): F1: 100% (210 nm), 99.6% (254 nm); F2: 96.3% (210 nm), 95.4% (254 nm). C₃₁H₃₆ClN₅O₃ (561.25).

Synthesis of 3^{rd} Generation Compound 8g/Gen C-10

Synthesis of tert-Butyl-4-(2-hydroxyethyl)piperazine-1-carboxylate 21

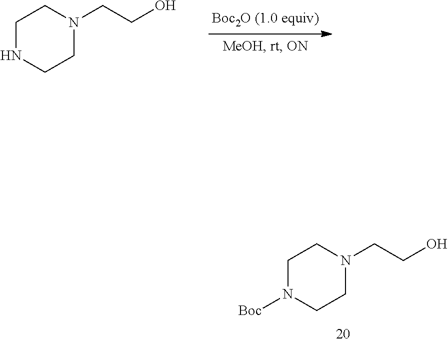 [SMILES: OCCN1CCNCC1 ; CC(C)(C)OC(=O)N1CCN(CCO)CC1]

According to the procedure of: I. Y. Lee, S. H. Sang, Y. H. Jung, Y. G. Kang, T. G. Oh, S. R. Cho, P. H. Kim, KR 2015060224, 2015: 582 mg (4.47 mmol, 1.0 equiv) 2-(piperazin-1-yl)ethan-1-ol were dissolved in 10 mL MeOH. 1.03 mL (976 mg, 4.47 mmol, 1.0 equiv) di-tert-butyl dicarbonate were added and the mixture was stirred at rt for 24 h. The solvent was removed in vacuo. 1.03 g (4.47 mmol, quant.) of compound 20 were isolated as colourless solid.

R_{F} (CH₂Cl₂/MeOH 5:1)=0.75. IR (film): v (cm⁻¹)=3441; 2975; 2934; 2868; 2813; 1697; 1457; 1420; 1366; 1291; 1247; 1171; 1128; 1077; 1055; 1004; 926; 866; 769. ¹H-NMR (500 MHz, CDCl₃): δ (ppm)=3.66-3.59 (m, 2H); 3.47-3.41 (m, 4H), 2.58-2.51 (m, 2H), 2.46 (t, J=5.1 Hz, 4H), 1.46 (d, J=0.7 Hz, 9H). HR-MS (ESI): calc. for C₁₁H₂₃N₂O₃ ([M+H]⁺): 231.1703; found: 231.1701. C₁₁H₂₂N₂O₃ (230.16).

### Synthesis of tert-Butyl-4-(2-methoxyethyl)piperazine-1-carboxylate 21

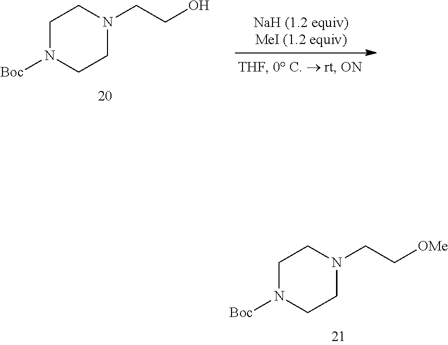 [SMILES: CC(C)(C)OC(=O)N1CCN(CCO)CC1 ; COCCN1CCN(C(=O)OC(C)(C)C)CC1]

In an oven-dried flask under nitrogen atmosphere, 87.8 mg (2.19 mmol, 1.2 equiv) sodium hydride were suspended in 10 mL THF and cooled to 0° C. 421 mg (1.83 mmol, 1.0 equiv) tert-butyl-4-(2-hydroxyethyl)piperazine-1-carboxylate 21 were added dropwise over a period of 10 min. The mixture was stirred at 0° C. for 1 h and 137 μL (312 mg, 2.19 mmol, 1.2 equiv) methyl iodide were added dropwise. After stirring at rt ON, 10 mL H₂O were added. The phases were separated and the aqueous layer was extracted with EtOAc (3×20 mL). The combined organic phases were dried over magnesium sulfate. The solution was filtered and the solvent was removed in vacuo. 425 mg (1.74 mmol, 95%) of compound 21 were isolated as a light yellow oil.

It was used as a novel approach for the synthesis of the literature-known and also commercially available compound 21.

R_{F} (CH₂Cl₂/MeOH 5:1)=0.93. IR (film): v (cm⁻¹)=2972; 2927; 2814; 1670; 1457; 1420; 1366; 1291; 1246; 1174; 1120; 1006; 868; 770. ¹H-NMR (500 MHz, CDCl₃): δ (ppm)=3.52 (t, J=5.5 Hz, 2H), 3.46 (t, J=5.1 Hz, 4H), 3.36 (s, 3H), 2.59 (t, J=5.6 Hz, 2H), 2.44 (t, J=5.0 Hz, 4H), 1.46 (s, 9H). HR-MS (ESI): calc. for C₁₂H₂₅N₂O₃ ([M+H]⁺): 245.1860; found: 245.1859. C₁₂H₂₄N₂O₃ (244.18).

### Synthesis of 1-(2-methoxyethyl)piperazine-2,2,2-trifluoroacetate 22

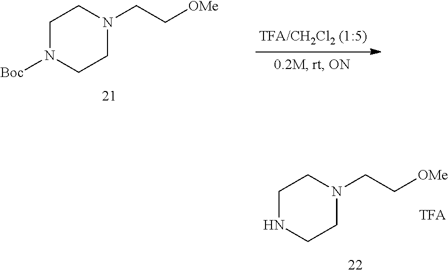 [SMILES: COCCN1CCN(C(=O)OC(C)(C)C)CC1 ; COCCN1CCNCC1]

According to the procedure of: J. F. Blake, S. A. Boyd, F. Cohen, J. De Meese, K. C. Feng, J. J. Gaudino, T. Kaplan, A. L. Marlow, J. Seo, A. A. Thomas, H. Tian, W. B. Young, WO 2007103308, 2007, but with a different procedure of isolation: tert-Butyl-4-(2-methoxyethyl)piperazine-1-carboxylate 21 (157 mg, 0.643 mmol, 1.0 equiv) was dissolved in 2.70 mL CH₂Cl₂ (0.2 M) at rt. 0.54 mL Trifluoroacetic acid (1:5 TFA/CH₂Cl₂) were added and the reaction mixture was stirred at rt ON. After completion of the reaction, the solvent was removed in vacuo. Residues of H₂O were removed by coevaporation with iPrOH (3×50 mL). 166 mg (0.644 mmol, quant.) of compound 22 were isolated as a yellow solid.

R_{F} (CH₂Cl₂/MeOH 5:1)=0.17. IR (film): v (cm⁻¹)=3012; 2845; 2730; 2488; 1671; 1459; 1322; 1131; 1016; 918; 835; 798; 722; 706. ¹H-NMR (400 MHz, CD₃OD): δ (ppm)=3.75-3.70 (m, 2H), 3.55 (s, 8H), 3.40 (s, 3H). HR-MS (ESI): calc. for C₇H₁₇N₂O ([M+H]⁺): 145.1335; found: 145.1334. C₇H₁₆N₂O (144.13). C₉H₁₇F₃N₂O₃ (258.12).

### Synthesis of N-(4-Hydroxy-3-((4-(2-methoxyethyl)piperazin-1-yl)methyl) phenyl)-acet-amide 10b

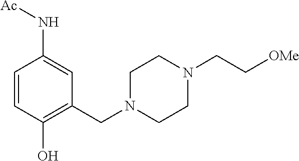 [SMILES: COCCN1CCN(Cc2cc(NC(C)=O)ccc2O)CC1]

The reaction was performed with 246 mg (1.63 mmol) acetaminophen, 815 μL (669 mg, 22.3 mmol) formaldehyde and 620 mg (2.4 mmol) 1-(2-methoxyethyl)piperazine-2,2,2-trifluoroacetate 22 in 16 mL iPrOH (in accordance with general procedure 6). Appearance: Light yellow oil. Yield: 280 mg, 0.913 mmol, 56%. R_{F} (CH₂Cl₂/MeOH 5:1)=0.55. C₁₆H₂₅N₃O₃ (307.19).

### Synthesis of 4-Amino-2-((4-(2-methoxyethyl)piperazin-1-yl)methyl)phenol 11b

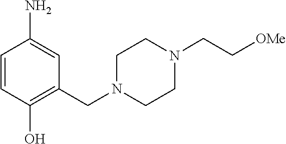 [SMILES: COCCN1CCN(Cc2cc(N)ccc2O)CC1]

The reaction was performed with 166 mg (0.541 mmol)N-(4-hydroxy-3-((4-(2-methoxyethyl)piperazin-1-yl)methyl)-phenyl)acetamide 10b in 1.4 mL 6 M HCl (in accordance with general procedure 7). Appearance: Light yellow oil. Yield: 97.6 mg, 0.368 mmol, 68%. R_{F} (CH₂Cl₂/MeOH 5:1)=0.68. IR (film): v (cm⁻¹)=3419; 3349; 3219; 2926; 2875; 2820; 632; 1498; 1457; 1404; 1346; 1307; 1245; 1219; 1199; 1155; 1118; 1010; 992; 932; 867; 814; 775. ¹H NMR (400 MHz, CDCl₃): δ (ppm)=6.65 (d, J=8.4 Hz, 1H), 6.55 (dd, J=8.4 Hz, J=2.8 Hz, 1H), 6.38 (d, J=2.8 Hz, 1H), 3.61 (s, 2H), 3.51 (t, J=5.5 Hz, 2H), 3.35 (s, 3H), 2.96-2.33 (m, 10H). HR-MS (ESI): calc. for C₁₄H₂₄N₃O₂ ([M+H]⁺): 266.1863; found: 266.1864. C₁₄H₂₃N₃O₂ (265.18).

### Synthesis of 4-((6-Chloro-2-methoxyacridin-9-yl)amino)-2-((4-(2-methoxyethyl)-piperazin-1-yl)methyl)phenol 8f/Gen C-10

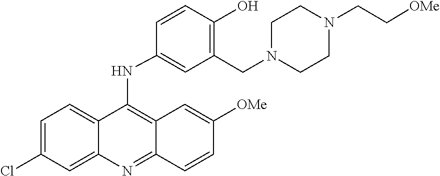 [SMILES: COCCN1CCN(Cc2cc(Nc3c4ccc(Cl)cc4nc4ccc(OC)cc34)ccc2O)CC1]

The reaction was performed with 89.4 mg (0.321 mmol) 6,9-dichloro-2-methoxyacridine and 85.2 mg (0.321 mmol) 4-amino-2-((4-(2-methoxyethyl)piperazin-1-yl)methyl)-phenol 11b in 6.5 mL EtOH (in accordance with general procedure 8). Appearance: Orange solid. Yield: 86.2 mg, 0.170 mmol, 53%. R_{F} (CH₂Cl₂/MeOH 5:1)=0.87. IR (film): v (cm⁻¹)=3296; 3054; 2940; 2880; 2823; 2763; 1631; 1605; 1561; 1518; 1493; 1437; 1420; 1362; 1345; 1334; 1306; 1254; 1235; 1156; 1119; 1072; 1031; 1011; 993; 928; 875; 829; 778; 736; 703; 665. ¹H-NMR (500 MHz, CDCl₃): δ (ppm)=8.13 (s, 1H), 8.03 (s, 1H), 7.90 (d, J=9.3 Hz, 1H), 7.40 (d, J=9.4 Hz, 1H), 7.08 (s, 1H), 6.84 (dd, J=8.6 Hz, J=2.7 Hz, 1H), 6.77 (d, J=8.6 Hz, 1H), 6.56 (d, J=2.7 Hz, 1H), 6.47 (s, 1H), 3.74 (s, 3H), 3.59 (s, 2H), 3.51 (t, J=5.5 Hz, 2H), 3.36 (s, 3H), 2.93-2.32 (m, 8H), 2.05-1.48 (m, 2H). HR-MS (ESI): calc. for C₂₈H₃₂ClN₄O₃ ([M+H]⁺): 507.2157; found: 507.2156. RP-HPLC-UV-Purity (C18 column, 0.1% TFA in H₂O/0.1% AA in MeCN): 96.3% (210 nm), 96.4% (254 nm). C₂₈H₃₁ClN₄O₃ (506.21).

2.3 Fifth Generation (Gen E)

2.3.1 Chemical Structures

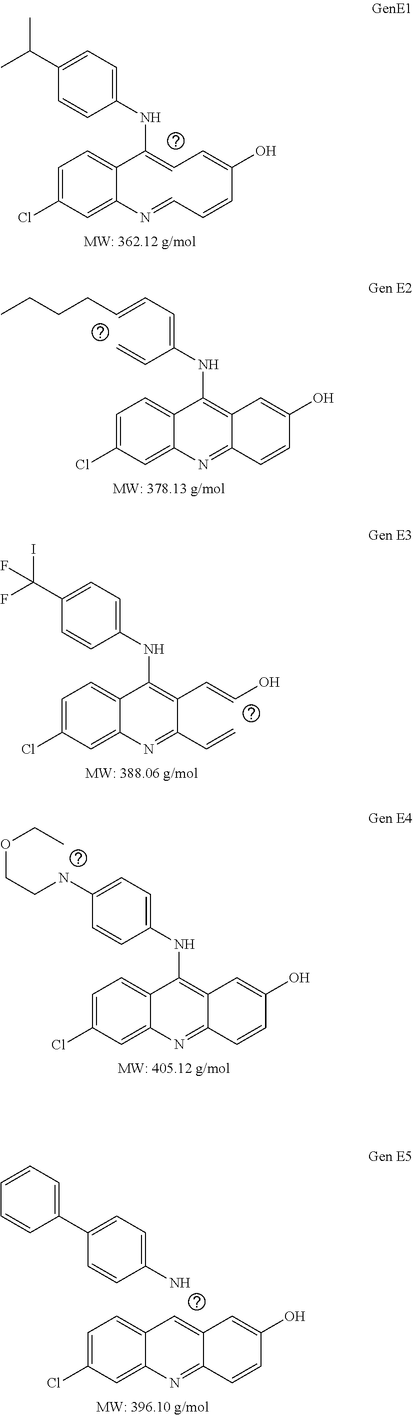

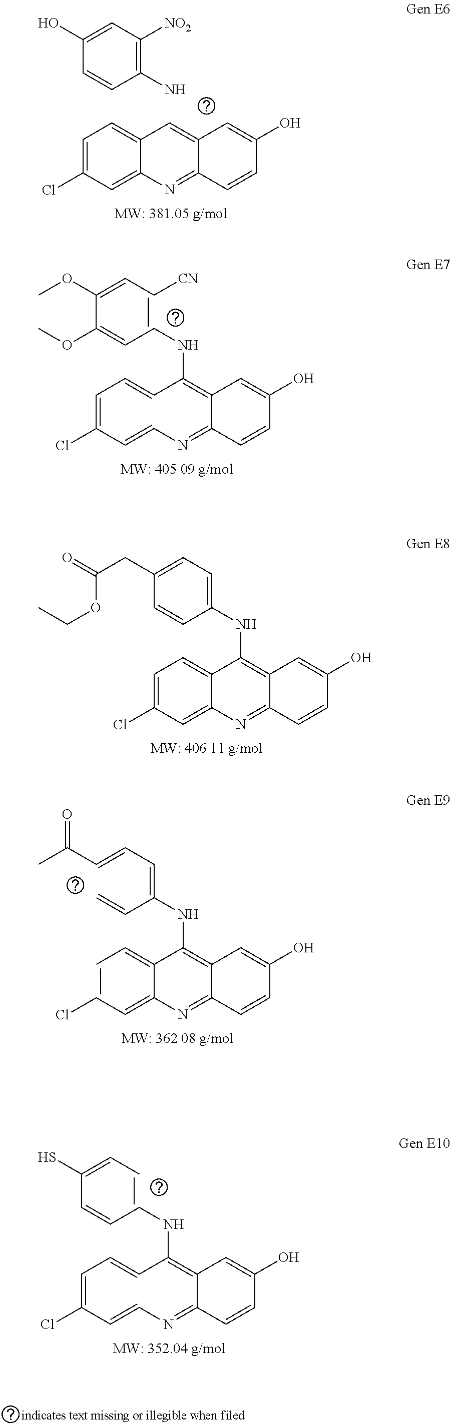

2.4 First Generation: Salt Forms

2.4.1 Chemical Structures of Gen A-4 (Salt Forms).

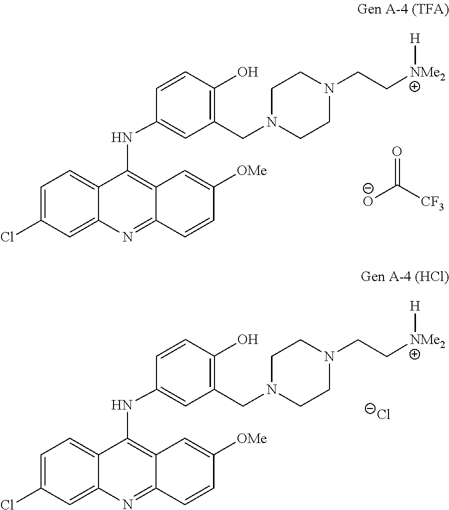

TABLE 2
F06-based analogues functionalized with different R substituents.

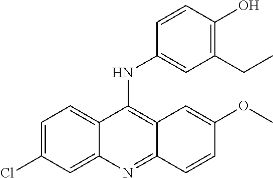 [SMILES: CCc1cc(Nc2c3ccc(Cl)cc3nc3ccc(OC)cc23)ccc1O]

| Compound | R | Yield |
|---|---|---|
| A1 (reference) | 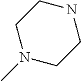 | 78% |
| A2 | 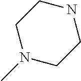 | 73% |
| A3 | 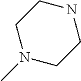 | 81% |
| A4 | 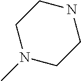 | 80% |
| A5 | 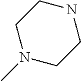 | 71% |
| A6 | 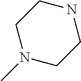 | 65% |
| A7 | 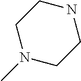 | 59% |
| A8 | | 73% |

TABLE 3
Pharmacophore features built from the docking
pose of F06 to the XPF site.

| Feature | Type | Radius (Å) |
|---|---|---|
| F1 | Aromatic | 1.5 |
| F2 | Aromatic | 1.5 |
| F3 | Aromatic | 1.5 |
| F4 | Donor | 3 |
| F5 | Aromatic | 1.5 |
| F6 | Acceptor | 2 |

TABLE 4
Computational results for the subset of analogues chosen for synthesis.
The ligand efficiency was calculated using the GBVI/WSA MOE score divided
for the number of heavy atoms (HA) of each molecule. The analogues are ranked
based on their MM/GBSA score which includes the entropy contributions.
The lead compound F06 is reported as the last entry.

| | GBVI/WSA | Ligand efficiency | | MM/GBSA |
|---|---|---|---|---|
| Compound | (kcal/mol) | ((kcal/mol)/HA) | logP | (kcal/mol) |
| A3 | −7.06 | −0.19 | 5.80 | −21.73 |
| A4 | −7.52 | −0.20 | 2.61 | −13.12 |
| A2 | −6.83 | −0.17 | 6.24 | −11.60 |
| A7 | −6.93 | −0.18 | 5.50 | −11.40 |
| A5 | −7.57 | −0.19 | 6.98 | −9.62 |
| A6 | −7.13 | −0.15 | 7.63 | −9.03 |
| A8 | −6.44 | −0.26 | 5.50 | −4.46 |
| A1 (F06) | −6.70 | −0.20 | 4.10 | −17.78 |

TABLE 5
Decomposition of total MM/GBSA binding energy in
different contributions for F06 and the two top analogues.

| | MM/GBSA (kcal/mol) | | | |
|---|---|---|---|---|
| | Van der | | Polar | Non-polar |
| Compound | Waals | Electrostatic | solvation | solvation |
| F06 | −44.55 | −12.69 | 21.35 | −3.41 |
| A3 | −43.09 | −25.99 | 29.10 | −3.47 |
| A4 | −43.76 | 63.57 | −51.31 | −3.12 |

TABLE 6
Half-maximum inhibitory concentrations for the compounds with
the highest inhibitory potentials. The data was obtained from
at least three different experiments of Vₒ versus
compound concentration.

| Compound | IC₅₀ ± SD (μM) | K_{d} ± SD (nM) |
|---|---|---|
| A1 (F06) | 1.86 ± 0.25 | 140 ± 5 |
| A3 | 0.38 ± 0.10 | 145 ± 5 |
| A4 | 0.33 ± 0.12 | 100 ± 5 |

### Example 4

Predicted Binding Mode of Gen B9 to the ERCC1-XPF Protein

In our previous work¹ we investigated the binding mode of F06 to XPF using molecular dynamics (MD) simulations in order to rationally design analogues with improved affinity. The virtual screening operation in our recent study² yielded compound Gen B9, where the piperazine ring of F06 was extended with an ethyl N,N-dimethyl group, and the acridine-methoxy group, a hydrogen bond acceptor, which was not engaged in any interaction, was replaced by a hydroxyl group, a hydrogen bond donor, resulting in the creation of an additional hydrogen bond with the side chain of the buried residue V859. These features contributed to lowering the binding energy and increased the ligand efficiency of Gen B9 compared to F06².

Potent Inhibitory Effect of Gen B9 and Gen C1 on the ERCC1-XPF

We have conducted a structure activity relationship analysis based around the previously identified hit compound F06 as a reference compound (FIG. 9). As previously reported, three different series of compounds have been rationally designed and successfully synthesized through various modifications on three different sites of F06 based on the corresponding suggestions of the previous pharmacophore model. The in vitro screening results revealed that Gen B9 has a potent inhibitory effect on the ERCC1-XPF activity with an IC₅₀ of 0.49 μM, showing ˜4-fold improvement in inhibition activity compared to F06 (Table 7).

TABLE 7
The half-maximum inhibitory concentrations (IC₅₀) and binding
constants values of inhibitors. The IC₅₀ data were obtained from at least
three different experiments of Vₒ versus compound concentration.

| Compound | K_{d} protein (nM) | K_{d} DNA (nM) | IC₅₀ (μM) |
|---|---|---|---|
| F06 (1) | 140 ± 5 | N.D.* | 1.86 ± 0.25 |
| Gen B7 | N.D. | 200 ± 10 | N.D. |
| Gen B9 | 85 ± 5 | 1050 ± 50 | 0.49 ± 0.04 |
| Gen D4 | 150 ± 10 | N.D. | 0.321 ± 0.022 |
| Gen D7 | N.D. | N.D. | 0.396 ± 0.051 |

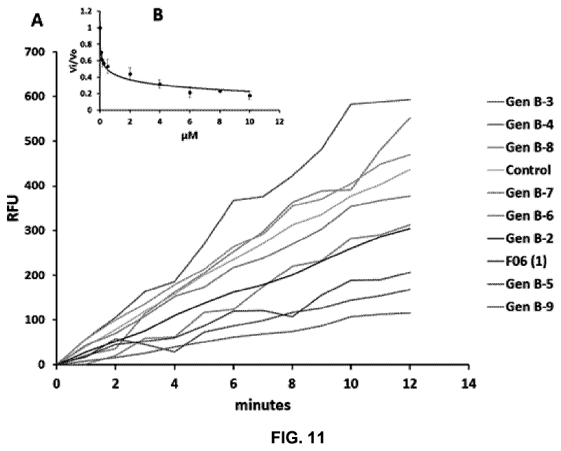

We also determined an IC50 value of 0.167+/−0.028 μM for compound Gen C-1 (FIG. 10).

Biochemical and Cell Based Assays of the ERCC1-XPF Inhibitors Lead Compounds

Inhibitory Effect of Gen B and Gen C Compounds on ERCC1-XPF Activity

As shown in FIG. 11, two compounds, Gen B5 and B9 exhibited a marked capacity to inhibit the nuclease activity of ERCC1-XPF compared to F06. The inhibitory activity of Gen B5 is superior to that of F06, which is probably attributable to the hydrogen bond donor phenolic group installed in replacement of the acridine-methoxy group of F06. Removal of the piperazine substituted aminophenol as in Gen B2 or acridine as in Gen B4 resulted in complete loss of inhibition. The IC50 of Gen B9 was estimated as 0.49±0.04 μM, which is approximately 4 times more efficient than F06, which has an estimated IC50 of 1.86±0.25 μM. The improved inhibitory activity of Gen B9 suggests the replacement of the acridine-methoxy group with a hydrogen bond donor group together with the conservation or extension of the piperazine ring enhances the activity of this generation of compounds.

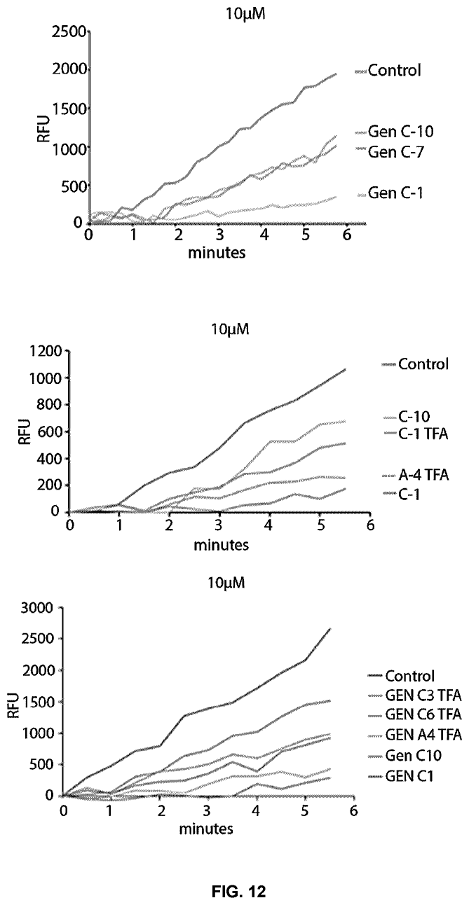

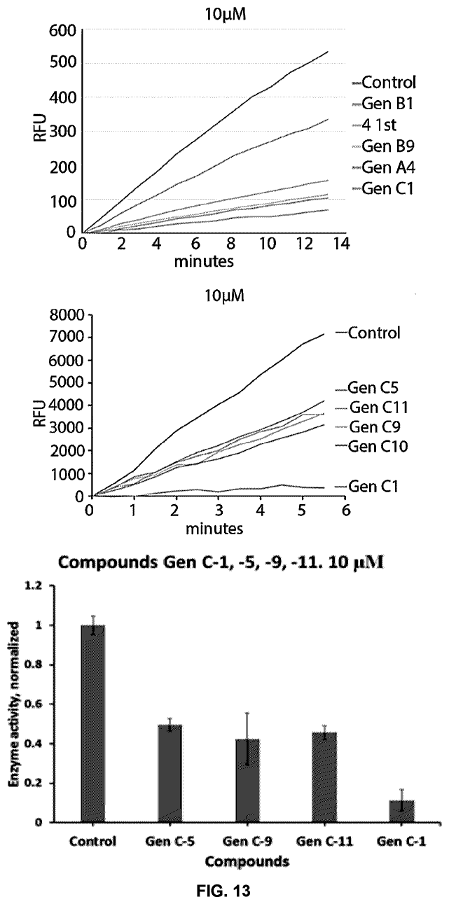

FIGS. 12 and 13 indicate the higher inhibitory effect of Gen C1 on the ERCC1-XPF endonuclease activity, observable through a lower increase in fluorescence by time in the in vitro real time fluorescent assay. Both experiments illustrate a further improvement in inhibitory activity, but subsequent analysis including an immunofluorescence UV based assay as well as a decomposition analysis of the free energy of binding will help to gain a better understanding about the new generation inhibitor Gen C1.

Use of Microscale Thermophoresis to Determine the Disruption of ERCC1-XPF by Gen A4

To identify a direct interaction between Gen A4 of the enzyme and ERCC1-XPF we utilized Micro Scale Thermophoresis (MST). MST allows for the detection of interactions and changes in the molecular properties of biomolecules at low nanomolar concentrations. We synthesized two human recombinant truncated forms of ERCC1 (dERCC1, codons 96-297) and XPF (dXPF, codons 667-916) for this study because these peptides contain the subunit interaction domains that the inhibitor was designed to disrupt. MST results showed that the drug induced a larger change in the thermophoresis properties of the truncated dimer as compared to each of the individual peptides indicating that the interaction of the compound with ERCC1-XPF altered the properties of the subunit interaction domain, which resulted in an inhibition of the endonuclease activity.

Cytotoxicity Profile of Inhibitors

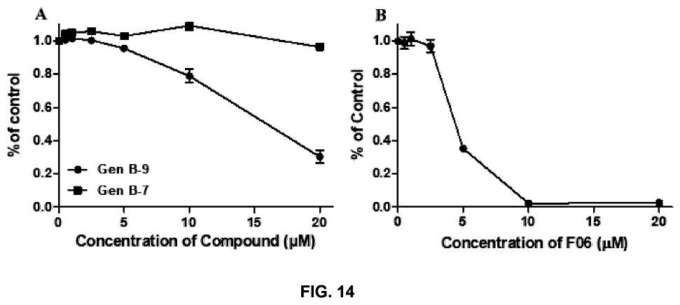

Crystal violet viability assays were carried out to assess the cellular toxicity of these compounds to HCT116 cells and to choose relatively non-cytotoxic concentrations to be further utilised in sensitizing cells to UV radiation or cyclophosphamide. As can be seen in FIG. 14, Gen B7 as a negative control is the least cytotoxic compound to HCT116 cells. The cytotoxicity of the hit F06 is greater than Gen B9, especially at concentrations≥5 μM. In the case of F06, about 35% of the cells survived after 3 days exposure of F06 to a dose of 5 μM, whereas approximately 95% of the HCT116 cells survived exposure to Gen B9 at the same dose. As a result, we selected 2 μM as a maximum low-cytotoxic concentration for F06, while 4 μM of Gen B9 and Gen B7 was chosen as a maximum concentration to be used in the following assay.

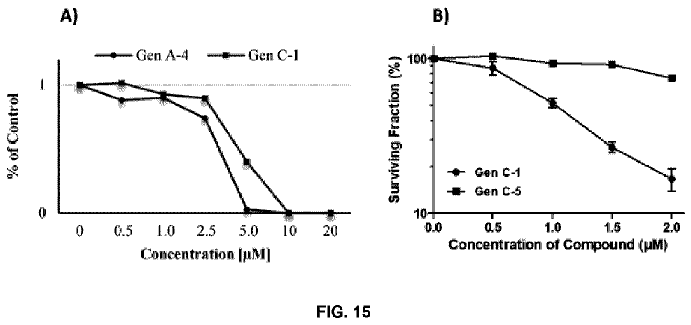

We assessed the cytotoxicity of compound Gen C1 and compared it with the first generation active compound Gen A4 by both using crystal violet staining and colony forming assay as shown in FIG. 15. Compound Gen C1 showed a lower cytotoxicity on HCT116 cell lines, compared to 1st generation compound Gen A4. The beneficial effect is especially visible at a concentration of 5.0 μM. At higher concentrations, both compounds show an equal cytotoxic effect (FIG. 15).

Increased Sensitisation to UV Radiation and Cyclophosphamide

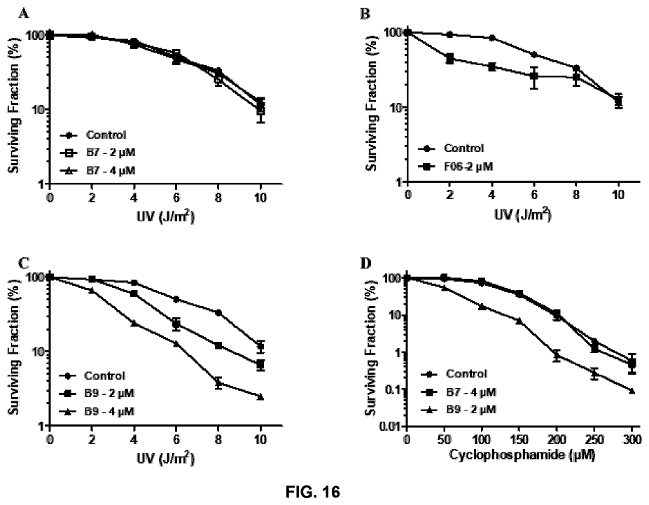

The effectiveness of Gen B9 to sensitise HCT116 cells to UV radiation and cyclophosphamide was evaluated. As depicted in FIG. 16A, neither 2 nor 4 μM Gen B7 (negative control) had a significant sensitisation effect on HCT116 cells to UV radiation, which is consistent with the failure of Gen B7 to inhibit ERCC1-XPF endonuclease. In contrast, 2 μM F06 displayed a modest sensitisation capacity (FIG. 16B), while 2 and 4 μM Gen B9 significantly reduced the survival of cells irradiated with UV (FIG. 16C). We then measured the effect of Gen B9 on the survival of cells exposed to the DNA interstrand cross-linking agent cyclophosphamide. The survival curves shown in FIG. 5D indicate that Gen B9, at a concentration of 2 μM, significantly sensitised the HCT116 cells to cyclophosphamide (starting at 50 μM) whereas there was no significant sensitisation effect with Gen B7 (at a higher dose of 4 μM).

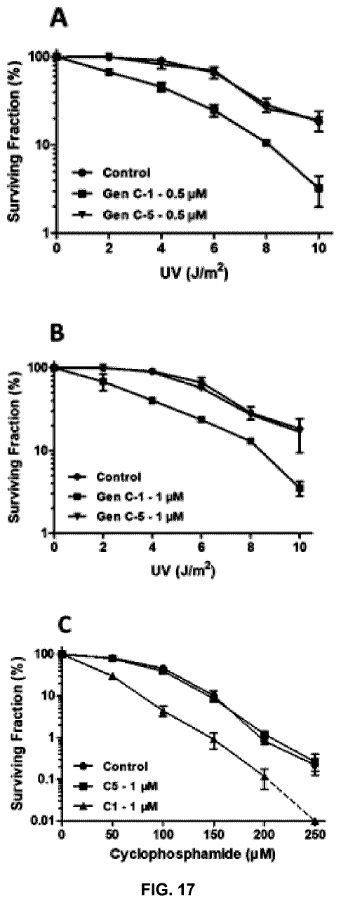

The Effectiveness of Gen C1 on sensitising HCT116 (wild type) to the UV radiation and cyclophosphamide was evaluated. As depicted in FIGS. 17A and B, Gen C1 at both 0.5 and 1 μM has a significant sensitisation effect on HCT116 cells to the UV radiation when compared to negative control Gen C5. Afterwards, the combined effect of Gen C1 (1 μM) on the survivals of cells after the exposure to cyclophosphamide, DNA interstrand cross-linking agent, using the wild type of HCT116. The survival curves shown in FIG. 17C indicate that Gen C1 significantly sensitised the HCT116 cells to cyclophosphamide (starting at 50 μM), whereas there is no significant sensitisation effect with Gen C5 which is in accordance with the UV survival data.

Cellular Repair of Cyclobutane Pyrimidine Dimers by Gen B9

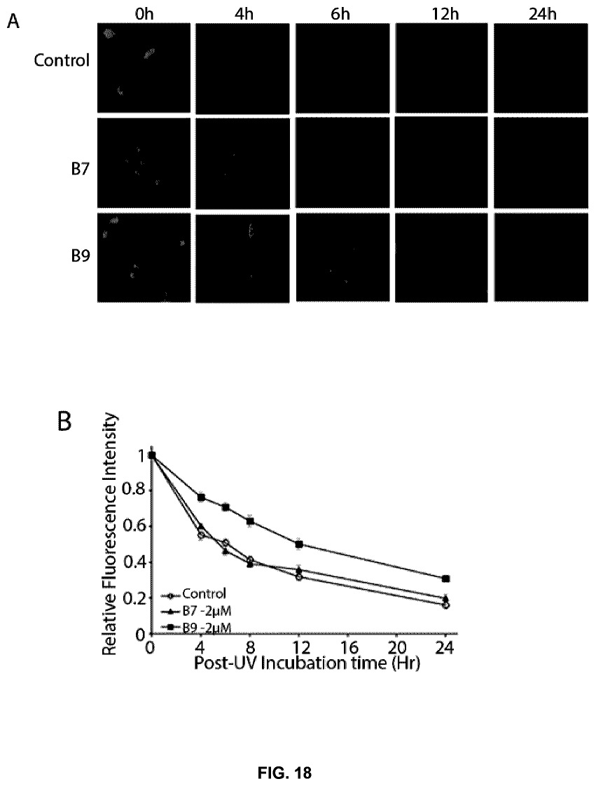

To test for inhibition of nucleotide excision repair, we monitored the cellular removal of UVC-induced cyclobutane pyrimidine dimers (CPDs) in HCT116 cells over a 24-hour period using an immunochemical assay. The results show that Gen B9 significantly inhibited the removal of CPDs compared with control cells or cells treated with Gen B7 (FIG. 18). 24 hours after irradiation the residual percentage of CPDs in the cells treated with Gen B7 was 35% compared to 16% in the untreated cells.

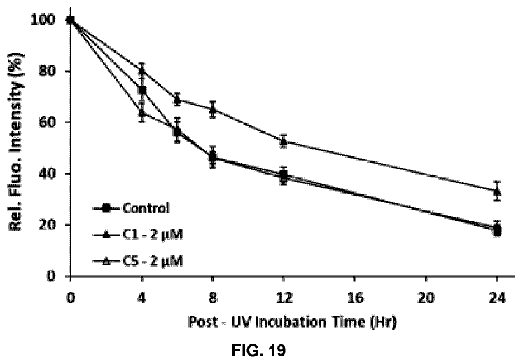

Significant inhibition of NER by Gen C1 determined by the removal of UV-induced CPDs compared with control cells over 24 hours is depicted in FIG. 19. Immunofluorescent detection of CPDs after the exposure of HCT116 colorectal cells to 8 J/m2 treated with Gen C1 showed a significant inhibition of removal of CPDs compared with control cells over 24 hours.

Pharmacokinetic Properties of A4, B9, C1 and D4 Inhibitors

We compared the results obtained from ADME properties to the parental compound F06 to determine if our compounds were pharmacokinetically superior to compound F06.

A4 showed a lower log D (2.86) than F06 (3.86) at pH 7.4 (Table 8). Metabolism of A4, as measured by exposure to human liver microsomes, indicated that it has a metabolism in the midrange, while F06 has a more rapid metabolism. Both A4 and F06 display an efflux ratio of higher than 2 (A4=8.92 and F06=11.18). Analysis of five CYP450 tested enzymes indicates that neither of the compounds has an IC50 lower than 1.00 μM; therefore they are not potent inhibitors of these enzymes. A4, however, moderately inhibits CYP1A2 with an IC50 of 6.40 μM. F06 has the same moderate inhibitory effect on CYP2D6 and CYP3A4-M with an IC50 between 1 and 10 μM. A4 acts as a weak inhibitor of CYP2D6 and CYP3A4-M, while F06 is a weak inhibitor of three CYP450 enzymes (CYP1A2, CYP2C9, and CYP2C19) with an IC50 between 10 and 50 μM. Both A4 and F06 have similar behavior in terms of permeability and binding to serum proteins as shown in Table 8.

Gen B9 showed (Table 8) a lower log D (2.01) than 1 (3.86) at pH 7.4. Metabolism of Gen B9, as measured by exposure to human liver microsomes, indicated that it has a moderate metabolism while compound F06 has a more rapid metabolism and high clearance from the liver. Both compounds display an efflux ratio greater than 2.

TABLE 8
ADME screening of the lead compounds inhibiting ERCC1-XPF complex

| Tests | F06 | Gen A-4 | Gen C-1 | B9 | D4 |
|---|---|---|---|---|---|
| LogD | Good | Good | Good | Good | Good |
| | 3.86 | 2.86 | 3.95 | 2.01 | 2.31 |
| | (partially soluble) | | (partially soluble) | | |
| Solubility (μM) | 8.13 | Poor | Good | Unstable | Highly soluble |
| [<1.56 means below | (partially soluble) | | | | |
| lower limit of | | | | | |
| quantilation] | | | | | |
| Cell permeability | Low (common) | Low | Low | Low | Low (common) |
| (Mean recovery %) | | | | (common) | |
| Serum protein | Strong binding | Strong | Strong binding | ND | ND |
| binding (% bound) | (99.9) | binding | (99.5) | | |
| | | (99.9) | | | |
| CVP inhibition | Moderate | Good | Good | ND | ND |
| (5 enzyme) | | | | | |
| Microsomal | 53.7 (high | Low | Low clearance | Moderate | Moderate |
| stability in vitro | clearance) | clearance | | | |
| CLint (ml/min/kg) | | | | | |
| Hepatocyte stability | <17.8 | 48.8 | 33.5 | ND | ND |
| in vitro CLint | (medium clearance) | (medium | (medium clearance) | | |
| (mL/min/kg) | | clearance) | | | |

Compounds Containing Alternative Moieties to Acridine

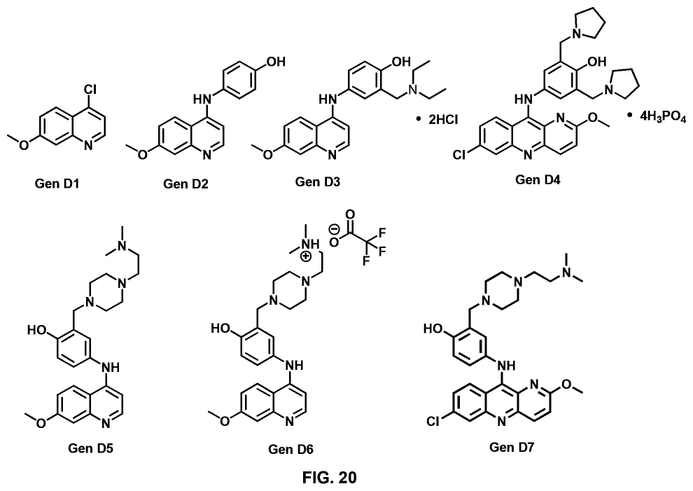

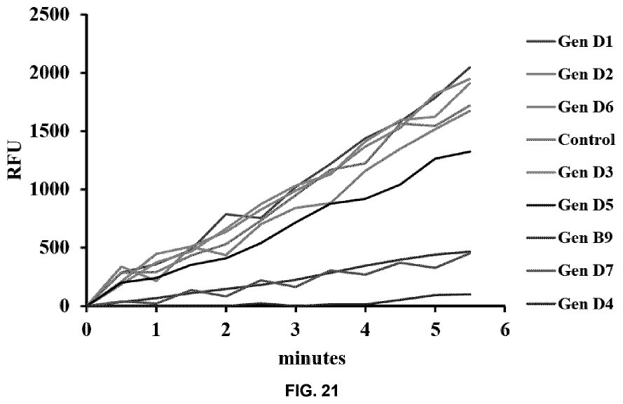

We synthesized a series of quinoline derivatives with the intention of replacing acridine so as to reduce the potential for binding to DNA. In addition, we identified pyronaridine (D4) as a compound with similar structure to our acridine-based compounds but containing a benzo[b][1,5]naphthyridine moiety in place of acridine (FIG. 20). D4 is commercially available and is a clinically approved anti-malarial compound. The results of ADME screening of this compound, together with our other lead compounds, is shown in Table 8. D4 appears to be a promising compound.

A series of quinoline derivatives were synthesized with the intention of replacing acridine so as to reduce the potential for binding to DNA. In addition, we identified pyronaridine (D4) as a compound with similar structure to our acridine-based compounds but containing a benzo[b][1,5]naphthyridine moiety in place of acridine (FIG. 20). D4 is commercially available.

Our standard fluorescence-based assay indicated that D4 in a potent inhibitor of ERCC1-XPF endonuclease activity, but the quinolone derivatives should almost know inhibition, with the possible exception of D5 (FIG. 21A). When compared with other three lead inhibitors (Gen-4, B9, and C1), D4 also showed a strong inhibitory effect (FIG. 21B).

Toxicity, UV Survival, UV Repair, and Cyclophosphamide Toxicity of the Lead Compounds

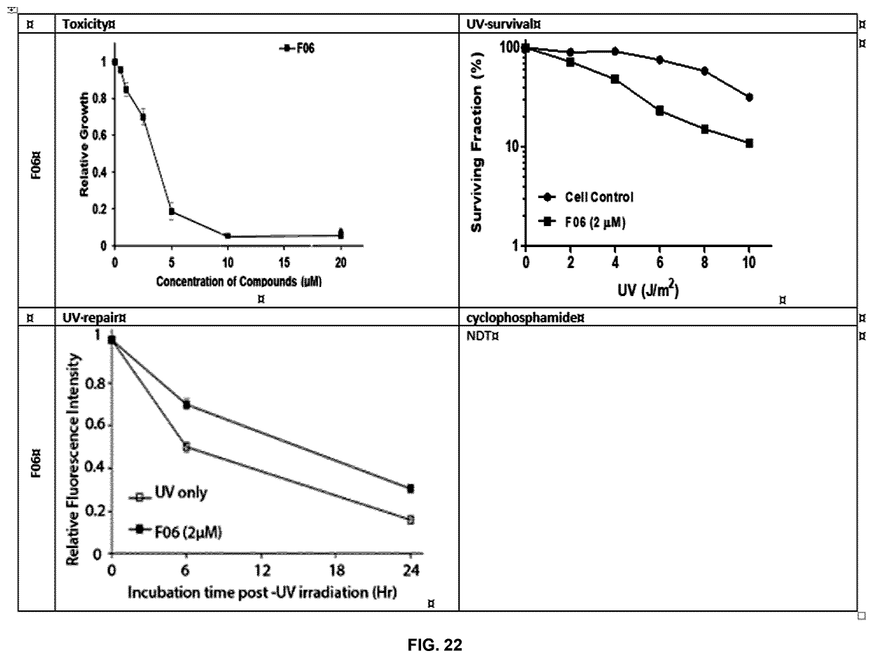

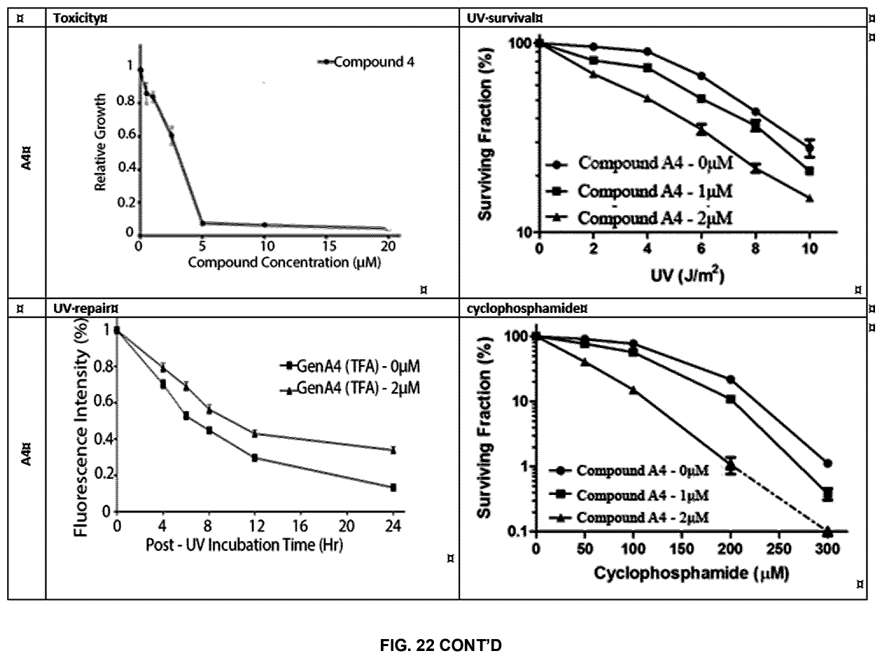

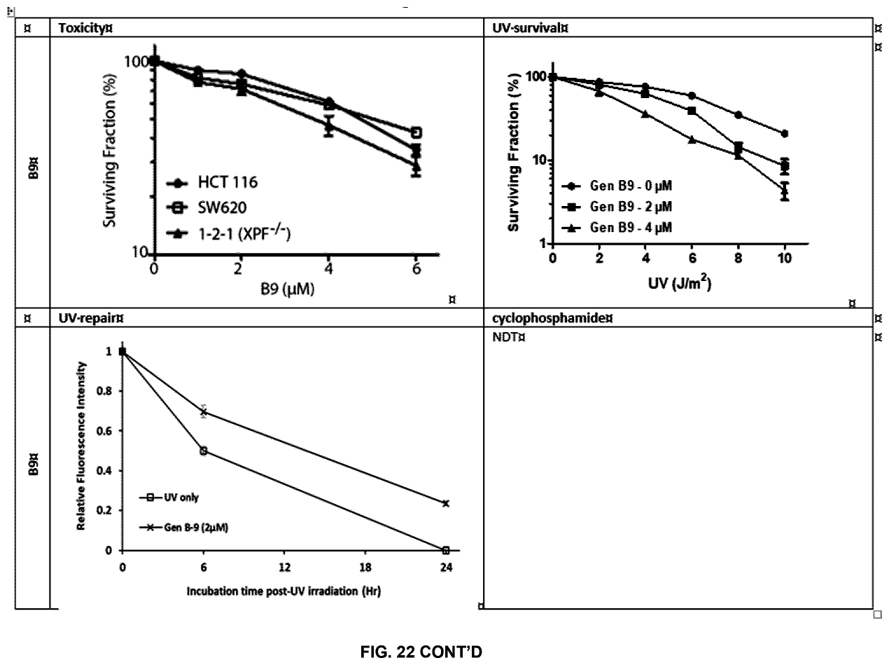

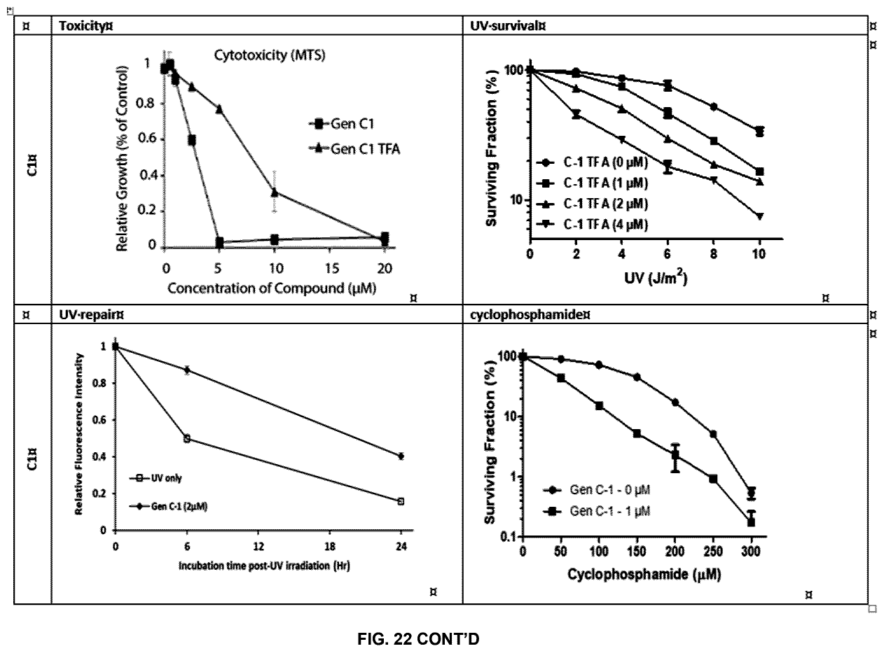

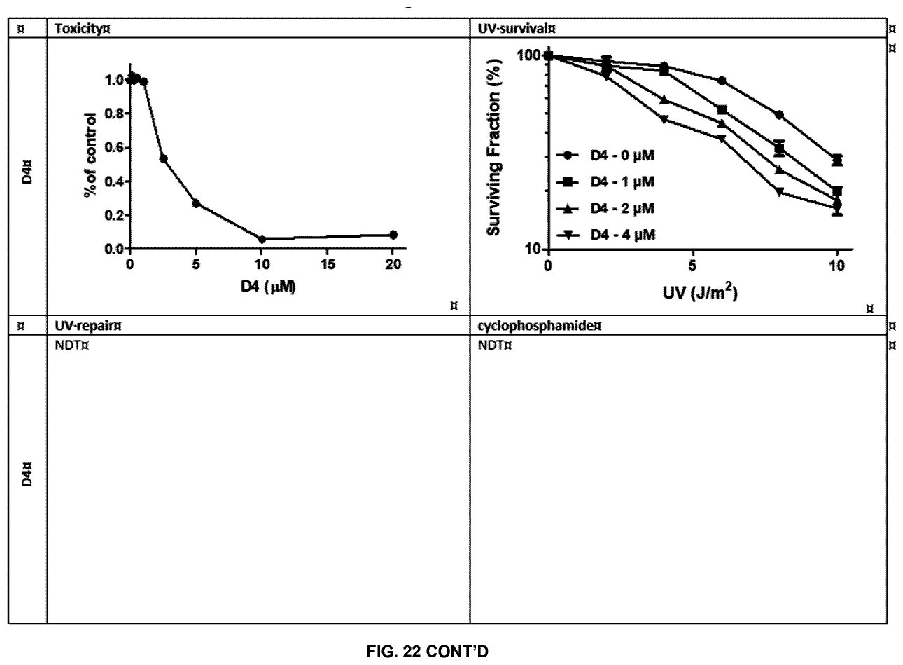

We evaluated the effectiveness of our four leading compounds (Gen A4, Gen B9, Gen C1, and D4) by four different cell based assays. The results are summarized in FIG. 22.

To evaluate the toxicity of the compounds we used MTS or crystal violet staining in order to gauge doses suitable for subsequent DNA repair and survival assays. The UV-survival of the cells was assessed by clonogenic survival assay. Cells were treated with 1 to 4 μM of the lead compounds and then exposed to increasing doses of UV radiation. To measure the inhibition of UV-repair we monitored the cellular removal of thymidine dimers by immunofluorescence. Our data as shown in FIG. 22 demonstrate that all four lead compounds are capable of significant inhibition of removal of thymidine dimers compared with control cells over 24 hours.

We then examined the effect of compounds Gen A4 and Gen C1 (1 and 2 μM) on cellular survival following exposure to the DNA interstrand crosslinking agent cyclophosphamide. The survival curves shown in FIG. 22 indicate that, at a concentration of 1 and 2 μM, compound Gen A4 and Gen C1 significantly sensitized the cells to cyclophosphamide.

The embodiments described herein are intended to be examples only.

Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

What is claimed is:

1. A compound of:
(a) Formula (I), 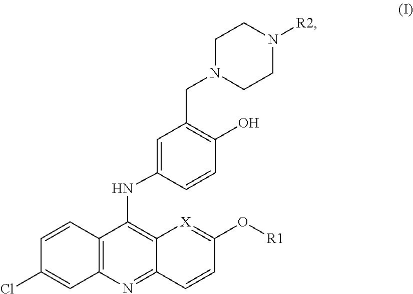
or a tautomer, a pharmaceutically acceptable salt, a solvate, or a functional derivative thereof;
wherein:
R¹ is H, short chain alkyl (Me, Et, iPr, nPr, nBu, iBu), substituted or unsubstituted phenyl, substituted or unsubstituted benzyl;
R² is a short chain alkyl (Me, Et, iPr, nPr, nBu, iBu), CH₂CH₂N(CH₃)₂, or CH₂CH₂N(CH(CH₃)₂)₂ substituted or unsubstituted phenyl, or substituted or unsubstituted benzyl, or C(Y)((CH₂)ₙ)N(R³R⁴), where n is 0, 1, 2, 3, or 4, and Y is O or S, and R³ and R⁴ are independently H, a short chain alkyl (Me, Et, iPr, nPr, nBu, iBu), substituted or unsubstituted phenyl, substituted or unsubstituted benzyl, or collectively form a 3-membered heterocyclic ring, 4-membered heterocyclic ring, a 5-membered heterocyclic ring, or a 6-membered heterocyclic ring, or ((CH₂)ₙ)N(R³R⁴), where n is 1, 2, 3, 4, or 5, and R³ and R⁴ are independently H, a short chain alkyl (Me, Et, iPr, nPr, nBu, iBu), substituted or unsubstituted phenyl, substituted or unsubstituted benzyl, or collectively form a 3-membered heterocyclic ring, 4-membered heterocyclic ring, a 5-membered heterocyclic ring, or a 6-membered heterocyclic ring;
X is either C or N; or
wherein when either R₁ or R₂ is an unbranched alkyl, the other is not an unbranched alkyl;
(b) Formula (II): 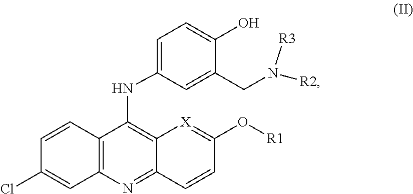
or a tautomer, a pharmaceutically acceptable salt, a solvate, or a functional derivative thereof;
wherein:
R¹ is H, a short chain alkyl (Me, Et, iPr, nPr, nBu, iBu), substituted or unsubstituted phenyl, substituted or unsubstituted benzyl;
R² and R³ are independently a short chain alkyl (Me, Et, iPr, nPr, nBu, iBu), substituted or unsubstituted phenyl, substituted or unsubstituted benzyl, or collectively form a 3-membered heterocyclic ring, 4-membered heterocyclic ring, a 5-membered heterocyclic ring, or a 6-membered heterocyclic ring;
X is either C or N; or
wherein when either R₁ or R₂ is an unbranched alkyl, the other is not an unbranched alkyl; or
(c) formula (III) 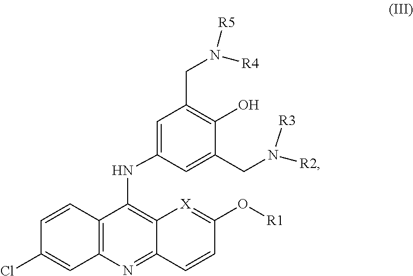
or a tautomer, a pharmaceutically acceptable salt, a solvate, or a functional derivative thereof;
wherein:
R¹ is H, a short chain alkyl (Me, Et, iPr, nPr, nBu, iBu), substituted or unsubstituted phenyl, substituted or unsubstituted benzyl;
R² and R³ are independently a short chain alkyl (Me, Et, iPr, nPr, nBu, iBu), substituted or unsubstituted phenyl, substituted or unsubstituted benzyl, or collectively form a 3-membered heterocyclic ring, 4-membered heterocyclic ring, a 5-membered heterocyclic ring, or a 6-membered heterocyclic ring;
R⁴ and R⁵ are independently a short chain alkyl (Me, Et, iPr, nPr, nBu, iBu), substituted or unsubstituted phenyl, substituted or unsubstituted benzyl, or collectively form a 3-membered heterocyclic ring, 4-membered heterocyclic ring, a 5-membered heterocyclic ring, or a 6-membered heterocyclic ring;
X is either C or N; and
wherein when R² and R³ collectively form a heterocyclic ring, R⁴ and R⁵ collectively do not form a heterocyclic ring.

2. The composition of claim 1, comprising: 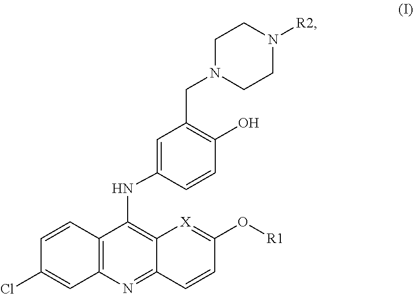
wherein R¹ is H, or CH₃
wherein R² is CH₃, CH₂CH₂N(CH₃)₂, or CH₂CH₂N(CH(CH₃)₂)₂, and
wherein X is C or N and
wherein when either R¹ or R² is CH₃, the other is not CH₃, or
a tautomer, or a pharmaceutically acceptable salt, or a solvate, or a functional derivative thereof.

3. The composition of claim 1, wherein R¹ is CH₃, R² is H and X is C.

4. The composition of claim 1, wherein R¹ is CH₂CH₂N(CH₃)₂, R² is H, and X is C.

5. The composition of claim 1, wherein R¹ is CH₂CH₂N(CH₃)₂, R² is CH₃, and X is C.

6. The composition of claim 1, wherein R¹ is CH₂CH₂N(CH(CH₃)₂)₂, R² is CH₃, and X is C.

7. The composition of claim 1, wherein CH₂CH₂N(CH₃)₂, R² is CH₃, and X is N

8. A method of treating a subject having cancer, or suspected of having cancer, comprising, administering a therapeutically effective amount of a compound of:
(a) Formula (I), 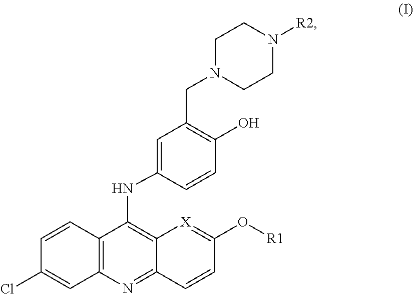
or a tautomer, a pharmaceutically acceptable salt, a solvate, or a functional derivative thereof;
wherein:
R¹ is H, short chain alkyl (Me, Et, iPr, nPr, nBu, iBu), substituted or unsubstituted phenyl, substituted or unsubstituted benzyl;
R² is a short chain alkyl (Me, Et, iPr, nPr, nBu, iBu), CH₂CH₂N(CH₃)₂, or CH₂CH₂N(CH(CH₃)₂)₂ substituted or unsubstituted phenyl, or substituted or unsubstituted benzyl, or C(Y)((CH₂)ₙ)N(R³R⁴), where n is 0, 1, 2, 3, or 4, and Y O or S, and R³ and R⁴ are independently H, a short chain alkyl (Me, Et, iPr, nPr, nBu, iBu), substituted or unsubstituted phenyl, substituted or unsubstituted benzyl, or collectively form a 4-membered heterocyclic ring, a 5-membered heterocyclic ring, or a 6-membered heterocyclic ring; or ((CH₂)ₙ)N(R³R⁴), where n is 1, 2, 3, 4, or 5, and R³ and R⁴ are independently H, a short chain alkyl (Me, Et, iPr, nPr, nBu, iBu), substituted or unsubstituted phenyl, substituted or unsubstituted benzyl, or collectively form a 4-membered heterocyclic ring, a 5-membered heterocyclic ring, or a 6-membered heterocyclic ring;
X is either C or N; or
wherein when either R₁ or R₂ is an unbranched alkyl, the other is not an unbranched alkyl;
(b) Formula (II): 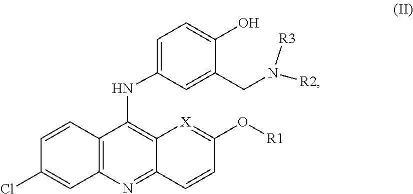
or a tautomer, a pharmaceutically acceptable salt, a solvate, or a functional derivative thereof;
wherein:
R¹ is H, a short chain alkyl (Me, Et, iPr, nPr, nBu, iBu), substituted or unsubstituted phenyl, substituted or unsubstituted benzyl;
R² and R³ are independently a short chain alkyl (Me, Et, iPr, nPr, nBu, iBu), substituted or unsubstituted phenyl, substituted or unsubstituted benzyl, or collectively form a 4-membered heterocyclic ring, a 5-membered heterocyclic ring, or a 6-membered heterocyclic ring;
X is either C or N; or
wherein when either R₁ or R₂ is an unbranched alkyl, the other is not an unbranched alkyl; or
(c) formula (III) 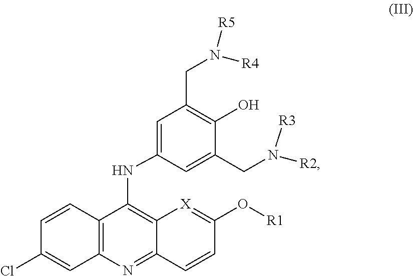
or a tautomer, a pharmaceutically acceptable salt, a solvate, or a functional derivative thereof;
wherein:
R¹ is H, a short chain alkyl (Me, Et, iPr, nPr, nBu, iBu), substituted or unsubstituted phenyl, substituted or unsubstituted benzyl;
R² and R³ are independently a short chain alkyl (Me, Et, iPr, nPr, nBu, iBu), substituted or unsubstituted phenyl, substituted or unsubstituted benzyl, or collectively form a 4-membered heterocyclic ring, a 5-membered heterocyclic ring, or a 6-membered heterocyclic ring;
R⁴ and R⁵ are independently a short chain alkyl (Me, Et, iPr, nPr, nBu, iBu), substituted or unsubstituted phenyl, substituted or unsubstituted benzyl, or collectively form a 4-membered heterocyclic ring, a 5-membered heterocyclic ring, or a 6-membered heterocyclic ring;
X is either C or N; and
wherein when R² and R³ collectively form a heterocyclic ring, R⁴ and R⁵ collectively do not form a heterocyclic ring.

9. The method of claim 8, comprising: 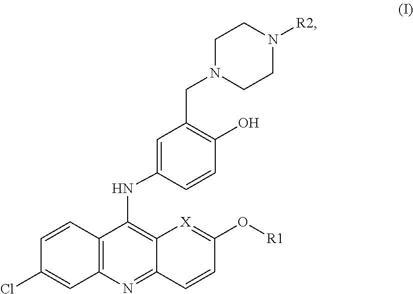
wherein R¹ is CH₃, CH₂CH₂N(CH₃)₂, or CH₂CH₂N(CH(CH₃)₂)₂, and
wherein R² is H, or CH₃,
wherein X is C or N wherein when either R¹ or R² is CH₃, the other is not CH₃,
or a tautomer, or a pharmaceutically acceptable salt, or a solvate, or a functional derivative thereof.

10. The method of claim 8, wherein R¹ is CH₃, and R² is H, and X is C.

11. The method of claim 8, wherein R¹ is CH₂CH₂N(CH₃)₂, and R² is H, and X is C.

12. The method of claim 8, wherein R¹ is CH₂CH₂N(CH₃)₂, and R² is CH₃, and X is C.

13. The method of claim 8, wherein R¹ is CH₂CH₂N(CH(CH₃)₂)₂, and R² is CH₃, and X is C.

14. The method of claim 8, wherein CH₂CH₂N(CH₃)₂, R² is CH₃, and X is N.

15. A method of treating a subject having cancer, or suspected of having cancer, comprising, administering a therapeutically effective amount of a compound of Formula (IV), 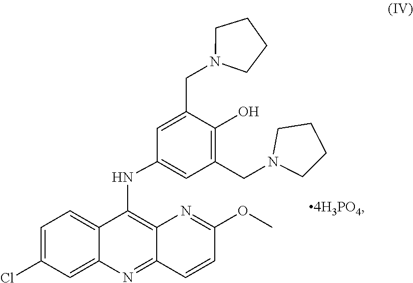 [SMILES: COc1ccc2nc3cc(Cl)ccc3c(Nc3cc(CN4CCCC4)c(O)c(CN4CCCC4)c3)c2n1]
or a pharmaceutically acceptable salt, or a solvate, or a functional derivative thereof.


SEQUENCE LISTING (1 sequence)

SEQ ID NO: 1 - DNA, 40 bp, Artificial Sequence
  stem-loop substrate
  6-FAM (1)..(1)
  dabcyl (40)..(40)
cagcgctcggttttttttttttttttttttccgagcgctg